(19) United States
(12) Patent Application Publication
(10) Pub. No.: US 2023/0279424 A1
(43) Pub. Date: Sep. 7, 2023

(54) COMBINATION CYTOKINES FOR METHODS AND COMPOSITIONS FOR TREATING CANCER

(71) Applicant: UNIVERSITY HEALTH NETWORK, Toronto (CA)

(72) Inventor: Christopher J. Paige, Toronto (CA)

(21) Appl. No.: 17/927,637

(22) PCT Filed: May 26, 2021
(86) PCT No.: PCT/CA2021/050711

§ 371 Date: Nov. 23, 2022

Related U.S. Application Data
(60) Provisional application No. 63/029,919, filed on May 26, 2020.

(51) Int. Cl. C12N 15/86; C07K 14/54; C07K 14/55; A61K 39/39; C12N 5/0783; A61P 35/00

(52) U.S. Cl. CPC C12N 15/86; C07K 14/5434; C07K 14/55; A61K 39/39; C12N 5/0636; A61P 35/00; A61K 2039/55538

(57) ABSTRACT

Methods and compositions of whole cell vaccines for delivering immune modulatory molecules IL-12 and at least one of IL-21 and/or IL-18 to result in a therapeutic effect are disclosed. The methods and compositions use stably integrating lentiviral delivery systems. The methods are useful for therapeutically and prophylactically treating cancer such as leukemia.

### RELATED APPLICATIONS

This Patent Cooperation Treaty application claims the benefit of priority of U.S. Provisional Application 63/029,919 filed May 26, 2020, which is incorporated herein in its entirety.

### INCORPORATION OF SEQUENCE LISTING

A computer readable form of the Sequence Listing “P61442PC00_ST25” (11,571 bytes) created on May 26, 2021, is herein incorporated by reference.

### FIELD OF INVENTION

The present disclosure relates generally to compositions and methods for therapeutically and prophylactically treating cancer. In particular, the present invention pertains to combinations of IL-12 and a second interleukin such as IL-21 or IL-18, lentiviral vectors encoding IL-12, IL-21, or IL-18 for transducing cells, and use of the transduced cells for cancer immunotherapy.

### BACKGROUND OF THE INVENTION

Cytokines have been candidates for use in anti-cancer immunotherapy protocols because of the well established regulatory properties they exert on immune responses. Clinical trials are underway using a variety of different cytokines including GM-CSF, IL-2, IL-7, IFN-γ, IL-12, IL-15, IL-18, IL-21, IL-23 and others. Current research seeks to optimize the dose, route and timing of delivery of these potent molecules.

The mature form of IL-12(p70) is composed of two subunits p35 and p40. IL-12 mediates both innate and adaptive immunity.^{1,2,15,16} The most abundant sources of IL-12 are activated dendritic cells (DCs) and macrophages.¹⁷ Secreted IL-12 promotes T helper cell type 1 (Th1) responses resulting in the production of IFNγ by T cells. IL-12 directly stimulates DCs resulting in increased maturation and antigen presentation, as well as the production of additional cytokines including TNFα, IL-8 and IL-10.3 NK and NKT cells are also activated by IL-12 to produce IFNγ.^{1,2}

IL-21 is a 17 kDa type 1 four-α-helical bundle cytokine primarily produced by Natural Killer T (NKT) cells and T cells.^{18,19} IL-21, acting alone or with other soluble factors, influences a wide range of immune system cells including enhanced proliferation and differentiation of naïve B cells, activation and production of IFNγ by Natural Killer (NK) cells, stimulation of cytotoxic programs in CD8⁺ T cells, and promoting switch of macrophage phenotype from M2 to M1, to name a few.¹⁸⁻²³

IL-18, originally called IFN-γ inducing factor (IGIF) when first discovered, is a member of the IL-1 superfamily.³³⁻³⁵ It is produced as a 24 kD pro-peptide that undergoes proteolytic cleavage by caspase-1 to generate the 18 kD active form. There are a number of potential sources of IL-18 including intestinal epithelial cells, osteoblasts, Kupfer cells as well as activated DCs and macrophages.^{36,37} IL-18 induces IFN-γ from a number of cells including T cells, NKT cells, NK cells DCs and macrophages. IL-18 promotes the differentiation of activated CD4 T cells towards Th-1 responses and activates NK cells. IL-18 has been shown to interact with IL-12 to increase levels of IFNγ expression.^{36,37} IL-18 binding protein, which normally is present at higher amounts than IL-18, antagonizes the effects of IL-18.38-4°

### Anti-Cancer Effects of Cytokines.

IL-12 has been utilized in a variety of preclinical and clinical settings both against solid tumors and leukemia.^{2,52-54} In humans, IL-12 based protocols have been tested in both advanced solid tumors and hematologic malignancies. The efficacy of IL-12 mono-therapy in the clinic for solid tumors has been minimal (0-11% objective response rate).² Better results have been obtained against cutaneous T cell lymphoma (56%),⁵⁵ Non-Hodgkin's lymphoma (21%)⁵⁶ and AIDS-related Kaposi sarcoma (71%).⁵⁷ IL-12 has generally failed in the clinic due to dangerous toxicity.¹⁻³ Systemic therapy often resulted in unacceptable levels of toxicity leading to decreased interest in the therapeutic potential of IL-12 gene therapy.⁵⁸⁻⁶³

Cell-based gene therapy has also been paired with other approaches including lipopolymerization of the IL-12 plasmid⁶⁴ and improved electroporation delivery methods,⁶³ targeted accumulation of IL-12 protein in the tumor microenvironment by creating immuno-cytokines that conjugate IL-12 to a TAA-specific monoclonal antibodies,⁶⁵ vaccination with tumor cell/DC fusions combined with low dose IL-12 administration,⁶⁶ and adoptive transfer of tumor-infiltrating lymphocytes (TILs) transduced to express IL-12 upon TCR-stimulation.^{62,67}

A number of early phase clinical trials have been carried out with IL-21.68-75 Objective responses were observed in phase 1 and 2 studies of IL-21 in melanoma and renal cell carcinoma.^{68,69}

IL-15 is an attractive potential partner for IL-12. In a recent murine study, prostate and breast cancer cells, transduced to secrete IL-15 or IL-15+IL-15Ra complex, were used to vaccinate mice.⁷⁸ While the complex was better than IL-15 alone, both procedures resulted in significantly prolonged survival in both tumor models. The effector cells were NK and CD8⁺ cells but not CD4⁺ cells. CD4⁺CTLs are induced by IL-12.^{4,6} IL-15 has also been paired with the checkpoint blockade inhibitors anti-CTLA4 and anti PD-L1 in a murine metastatic colon carcinoma model.⁷⁹ While there was significant reduction in lung metastasis, and prolonged survival, with IL-15 alone, the combination of IL-15 with either anti-CTLA4 or anti PD-L1 was significantly better. There have also been several experimental immunotherapy studies pairing IL-12 and IL-15. In one study using a human small cell lung cancer cell line in a nude mouse xenograft model it was shown that these cytokines synergize to produce a protective Antibody Dependent Cell mediated cytotoxicity (ADCC) response.¹⁰ In another study in mice bearing B16F10 melanoma cells received systemic injections of IL-12+IL-15 which resulted in protection mediated by NK, CD8⁺CTL, IFNγ, and activated macrophages.⁸⁰

IL-18 cytokine administration is an effective anti-cancer agent in a number of murine models both alone and in conjunction with other immune mediators.⁸²⁻⁸⁵ Murine models have also shown anti-cancer effects when combined with check point blockers such as anti-PD-L1 or anti-CTLA 4.⁸⁶ In several models the combination of IL-12 and IL-18 as bolus injection proved toxic for mice.⁸⁸ Human Phase 1 trials have also been undertaken using either IL-18 alone or in combination with rituximab.⁸⁹⁻⁹¹

IL-7 has shown anti-cancer activity in murine models of lung cancer and sarcoma.^{92,93} It was shown to increase the effectiveness of CTL in a murine model.⁹² IL-7 has also been used in a variety of settings in human studies. It enhanced the proliferation of CAR-T cells.⁹⁴ As a monotherapy in human clinical trial it has been used with a number of advanced cancers showing significant increases in CD4 and CD8 T cells and a decrease in T regulator cells.^{96,96} IL-12 has been shown to act directly on CD8T cells to enhance their IL-7 responsiveness. IL-7 and IL-12 also show synergy in IFNγ responses from virus specific CD8+ T cells.¹⁴ IL-12 and IL-7 work synergistically to enhance Granzyme b expression by T cells in a human lung anti-bacterial model.⁹⁷ IL-12 and IL-7 have recently been shown to synergize in an preclinical oncolytic viral model to enhance sensitivity to checkpoint blockade in multiple murine cancers.⁹⁸

WO/2008/134879, incorporated herein by reference, describes recombinant cells expressing IL-12 above a threshold level, lentiviral vectors encoding IL-12 for transducing cells, and use of the transduced cells for cancer immunotherapy. Levels of IL-12 production and percentages of IL-12 producing cells required to elicit an effective immune response are also described. IL-12 has been introduced and demonstrated to elicit immune responses in a variety of leukemia and solid tumor models.⁴⁻⁸ Related thereto is the Phase 1 study of autologous acute myelogenous leukemia (AML) cells containing lentivirus engineering expression of IL-12: NCT02483312.

Barrett et al. [145] describes adenoviral vectors encoding IL-12 under control of a Rheo-Switch Therapeutic System (RTS) gene switch, inducible by the activator veledimex (VDX), and use of the adenoviral vectors for cancer immunotherapy. Dose-dependent increases of IL-12 and IFN-γ following VDX treatment of mice dosed with the adenoviral vectors in glioma models are described. A subsequent Phase 1 study (NCT02026271) by Chiocca et al [146] describes the treatment of patients undergoing resection of recurrent high-grade glioma. Dose-dependent increases in VDX, IL-12, and IFN-γ in peripheral blood are described, with about 40% tumor penetration. Increased tumor-infiltrating lymphocytes producing IFN-γ and programmed cell death protein 1 (PD-1) are described in patients with pseudoprogression.

Improved cancer treatments are desirable.

### SUMMARY

Experimental cancer models demonstrate that the cytokines noted above have pleotropic effects on the immune response with many overlapping, and potentially complimentary, functions. IL-12 was selected as one component of a cytokine combination since preclinical and clinical models for IL-12 solo therapy have been established, providing a baseline upon which to improve.⁴⁻⁷ Cells secreting high levels of IL-12 (eg 10 ng/ml/10⁶ cells/hr) have been shown to result in immunity even if they represent only 0.5% of the total leukemia cells injected.⁴ Other clones secreting lower amounts of IL-12 failed to establish immunity in all mice even if they represented 10% of the injected cells (but do so if 100% of cells are expressing the cytokine).⁴⁻⁷ This model provides us with an experimental approach to improve immunity with lower expressing clones. This is important because transducing human leukemia cells with for example, a lentivirus vector results in a broad range of expression levels in individual cells.⁸ Based on transductions of more than 30 individual primary human AML samples a range of cytokine secretion levels was observed within a transduced population, from very low to very high (<1 to >50 ng/ml/10⁶ cells/hr).⁸

Accordingly, the transduction of a population of patient cells may result in a range of expression levels, wherein for example only a percentage of cells may produce IL-12 above the threshold level required to be recognized by the patient immune system and thus elicit protective immunity. Furthermore, transduction efficiencies may be low and/or it may be difficult to obtain sufficient patient cells for transduction (e.g. blast cells) from patients, particularly from patients with active but stable disease.

It was hypothesized that clinical impact could be increased by improving the immune stimulating capacity of cells in the lower range of IL-12 expression for example those transduced cells expressing IL-12 below the threshold level required to initiate an effective anti-cancer immune response. Using their preclinical murine model, the inventors selected clones secreting lower amounts of IL-12 for cytokine combination experiments.

It was also reasoned that the second cytokine should target receptors distinct from the IL-12 receptor to increase the breadth of immune reactions. Cytokines utilizing the common γ chain including IL-7, 15, and 21 as well as the IL-1 family member IL-18 fulfilled this requirement and have been used in the clinic already. As demonstrated herein, IL-18 and IL-21 but not IL-7 or IL-15 increased the efficacy of IL-12 low expressing cells.

Accordingly, one aspect of the disclosure includes a composition comprising: a multicytokine lentiviral construct comprising: a lentiviral vector; an IL-12 expression cassette; and an IL-21 expression cassette and/or an IL-18 expression cassette. In an embodiment, the IL-21 expression cassette and the IL-12 expression cassette form an IL-12-IL-21 expression cassette. In an embodiment, the IL-21 expression cassette and the IL-12 expression cassette form an IL-21-IL-12 expression cassette. In an embodiment the IL-18 expression cassette and the IL-12 expression cassette form an IL-12-IL-18 expression cassette. In an embodiment the IL-18 expression cassette and the IL-12 expression cassette form an IL-18-IL-12 expression cassette.

In an embodiment, the IL-12 expression cassette comprises a polynucleotide optionally encoding a p35 polypeptide and a polynucleotide encoding a p40 polypeptide, or a polynucleotide encoding an IL-12 fusion polypeptide. In an embodiment, the polynucleotide encoding the IL-12 fusion polypeptide has at least 70% sequence identity to SEQ ID NO: 4 and binds an IL-12 receptor.

In an embodiment, the IL-21 expression cassette and the IL-12 expression cassette form an IL-12-IL-21 expression cassette or IL-21-IL-12 expression cassette. In an embodiment, the IL-18 expression cassette and the IL-12 expression cassette form an IL-12-IL-18 expression cassette or IL-18-IL-12 expression cassette.

In an embodiment, the IL-21 expression cassette encodes an IL-21 polypeptide having at least 70% sequence identity to SEQ ID NO: 7 and binds an IL-21 receptor.

In an embodiment, the IL-18 expression cassette encodes an IL-18 polypeptide having at least 70% sequence identity to SEQ ID NO: 6 and binds an IL-18 receptor.

In an embodiment, one or more of the IL-12 expression cassette, and the IL-21 expression cassette and/or the IL-18 expression cassette comprises an inducible promoter.

In an embodiment, the lentiviral vector is a clinical grade vector.

In an embodiment, the composition is a pharmaceutical composition and further comprises a pharmaceutically acceptable carrier.

An aspect of the disclosure includes a vector construct comprising: a lentiviral vector; an IL-12 expression cassette; and an IL-21 expression cassette and/or an IL-18 expression cassette. In an embodiment, the IL-21 expression cassette and the IL-12 expression cassette form an IL-12-IL-21 expression cassette or IL-21-IL-12 expression cassette. In an embodiment, the IL-18 expression cassette and the IL-12 expression cassette form an IL-12-IL-18 expression cassette or IL-18-IL-12 expression cassette.

A further aspect of the disclosure includes an isolated virus comprising a vector construct or composition comprising: a lentiviral vector; an IL-12 expression cassette; and an IL-21 expression cassette and/or an IL-18 expression cassette. In an embodiment, the IL-21 expression cassette and the IL-12 expression cassette form an IL-12-IL-21 expression cassette or IL-21-IL-12 expression cassette. In an embodiment, the IL-18 expression cassette and the IL-12 expression cassette form an IL-12-IL-18 expression cassette or IL-18-IL-12 expression cassette.

A further aspect of the disclosure includes an isolated cell, preferably a cancer cell, secreting IL-12 and at least one of IL-21 and/or IL-18 at or above a threshold level. In an embodiment, the cell is transduced with a composition, vector construct, or isolated virus comprising: a lentiviral vector; an IL-12 expression cassette; and an IL-21 expression cassette and/or an IL-18 expression cassette. In an embodiment, the IL-21 expression cassette and the IL-12 expression cassette form an IL-12-IL-21 expression cassette or IL-21-IL-12 expression cassette. In an embodiment, the IL-18 expression cassette and the IL-12 expression cassette form an IL-12-IL-18 expression cassette or IL-18-IL-12 expression cassette. In an embodiment the cancer is leukemia, lymphoma, myeloma, glioblastoma, melanoma, or cancer of the lung, ovary, prostate, breast, colon, bladder, liver, pancreas, thyroid, or head and neck.

In an embodiment, the cell is a cancer cell, optionally an established cell line, optionally a primary cancer cell, optionally a cancer cell derived from a subject. In an embodiment the cancer cell is a leukemic cell, optionally an ALL cell, an AML cell or a CLL cell. In an embodiment, the cell is an autologous cell. In an embodiment, the cell is an allogenic cell, optionally the allogenic cell is a cancer cell of the same type as the cancer being treated.

As demonstrated in the Examples, co-expression of IL-18 or IL-21 in cells expressing a low level of IL-12, for example expressing about 2000 pg/ml/10⁶ cells/hr and less than for example 10 000 pg/ml/10⁶ cells/hr increased the percent survival of animals that were administered leukemic cells that secreted IL-12 and IL-18 compared to animals administered control cells.

In an embodiment, the IL-12 is secreted at a ratio of at least or about 10:1, at least or about 5:1, at least or about 2:1, or at least or about 1:1 relative to IL-21 or IL-18. In an embodiment, the IL-12 is secreted at a ratio of between 10:1 and 1:1, between 10:1 and 2:1, between 10:1 and 5:1, between 5:1 and 1:1, between 5:1 and 2:1, or between 2:1 and 1:1 relative to IL-21 or IL-18.

A further aspect includes a population of cells comprising isolated cells comprising a lentiviral vector; an IL-12 expression cassette; and an IL-21 expression cassette and/or an IL-18 expression cassette.

In an embodiment, the population of cells, preferably cancer cells, optionally comprises at least 0.1 to 1% IL-12 and at least one of IL-21 and/or IL-18 producing cells, optionally about 0.5%, about 1%, about 1-5%, 5-10%, 10-20%, 20-30%, 30-40%, 40-50%, 50-60%, 60-70%, 70-80%, 80-90%, 90-95%, 95-99% or more than 99% IL-12 and at least one of IL-21 and/or IL-18 producing cells, and wherein the population of cells secretes IL-12 and at least one of IL-21 and/or IL-18 levels necessary to induce or enhance an immune response in a subject, for example a CD4+ T cell dependent immune response, a CD8 T cell dependent immune response, a natural killer (NK) dependent immune response, and/or a gamma delta T cell dependent immune response. In an embodiment, the population of cells comprises at least 10% or at least 20% IL-12 and at least one of IL-21 and/or IL-18 producing cells. In an embodiment the population of cells is a population of cancer cells, optionally the cancer is leukemia, lymphoma, myeloma, glioblastoma, melanoma, or cancer of the lung, ovary, prostate, breast, colon, bladder, liver, pancreas, thyroid, or head and neck.

In an embodiment, the population of cells comprises at least 1%, optionally at least 2%, at least 5%, at least 10%, at least 20% or more cells secreting at least 500 pg/10⁶ cells/ml/hr 1,000 pg/10⁶ cells/ml/hr, 1,500 pg/10⁶ cells/ml/hr, 2,000 pg/10⁶ cells/ml/hr, 2,500 pg/10⁶ cells/ml/hr, 5,000 pg/10⁶ cells/ml/hr, 7,500 pg/10⁶ cells/ml/hr, 10,000 pg/10⁶ cells/ml/hr, 12,500 pg/10⁶ cells/ml/hr, 15,000 pg/10⁶ cells/ml/hr, 17,500 pg/10⁶ cells/ml/hr or 20,000 pg/10⁶ cells/ml/hr of IL-12. In an embodiment, the population of cells comprises at least 1%, optionally at least 2%, at least 5%, at least 10%, at least 20% or more cells secreting 500-1000 pg/10⁶ cells/ml/hr 1,000-1,500 pg/10⁶ cells/ml/hr, 1,500-2,000 pg/10⁶ cells/ml/hr, 2,000-2,500 pg/10⁶ cells/ml/hr, 2,500-5,000 pg/10⁶ cells/ml/hr, 5,000-7,500 pg/10⁶ cells/ml/hr, 7,500-10,000 pg/10⁶ cells/ml/hr, 10,000-12,500 pg/10⁶ cells/ml/hr, 12,500-15,000 pg/10⁶ cells/ml/hr, 15,000-17,500 pg/10⁶ cells/ml/hr, or 17,500-20,000 pg/10⁶ cells/ml/hr of IL-12.

Different percentages of cells secreting the cytokines combined with different concentration of secreted cytokine are contemplated herein, in compositions and the like and for use to induce or enhance an immune response in a subject. For example, the population of cells can comprise a percentage between least 1% and less than 50% IL-12+IL-18/IL-21 secreting cells. The concentration of IL-12 secreted can for example be any concentration between for example 500 pg/10⁶ cells/ml/hr and 10000 pg/10⁶ cells/ml/hr.

For example, the population of cells can comprise at least 10%, at least 20%, at least 30%, at least 40%, or at least 50% of cells secreting about 2,000 pg/10⁶ cells/ml/hr IL-12 and about 2,000 pg/10⁶ cells/ml/hr IL-18. Other combinations are contemplated.

In an embodiment, the population of cells comprises at least 1% of cells expressing at least 10,000 pg/10⁶ cells/ml/hr IL-12 and at least 5,000 pg/10⁶ cells/ml/hr IL-18. In an embodiment, the population of cells comprises at least 5% of the combination cytokine secreting cells. In an embodiment, the population of cells comprises at least 10% of such cells. In an embodiment, the population of cells comprises at least 15% of such cells. In an embodiment, the population of cells comprises at least 20% of such cells. In an embodiment, the population of cells comprises at least 25% of such cells. In an embodiment, the population of cells comprises at least 30% of such cells. In an embodiment, the population of cells comprises at least 35% of such cells. In an embodiment, the population of cells comprises at least 40% of such cells. In an embodiment, the population of cells comprises at least 45% of such cells. In an embodiment, the population of cells comprises at least 50% of such cells.

In an embodiment, the population of cells, preferably cancer cells, optionally comprises at least 0.1×10⁶ IL-12 and at least one of IL-21 and/or IL-18 producing cells, optionally about 1×10⁶, about 2×10⁶, about 3×10⁶, about 4×10⁶, about 5×10⁶, about 6×10⁶, about 7×10⁶, about 8×10⁶, about 9×10⁶, about 10×10⁶, about 15×10⁶, about 20×10⁶, or more IL-12 and at least one of IL-21 and/or IL-18 producing cells, and wherein the population of cells secretes IL-12 and at least one of IL-21 and/or IL-18 levels necessary to induce or enhance an immune response in a subject, for example a CD4+ T cell dependent immune response, a CD8 T cell response, an NK response, and/or a gamma delta T cell response. Optionally the population comprises about 0.1-05×10⁶, about 0.5-1×10⁶, about 1-2×10⁶, about 2-3×10⁶, about 3-4×10⁶, about 4-5×10⁶, about 5-6×10⁶, about 6-7×10⁶, about 7-8×10⁶, about 8-9×10⁶, about 9-10×10⁶, about 10-15×10⁶, or about 15-20×10⁶ IL-12 and at least one of IL-21 and/or IL-18 producing cells.

The number of cells secreting the cytokines combined with different concentration of secreted cytokines can be used to induce or enhance an immune response in a subject, and are contemplated herein. For example, at least 1×10⁶ IL-12+IL-18/IL-21 secreting cells, optionally about 2×10⁶, about 3×10⁶, about 4×10⁶, about 5×10⁶ IL-12+IL18/IL-21 secreting cells, may be effective for inducing or enhancing an immune response in a subject when cells are expressing levels of IL-12 below for example 2,000 pg/10⁶ cells/ml/hr. In another example, fewer than 1×10⁶ IL-12+IL18/IL-21 secreting cells may be effective for inducing or enhancing an immune response in a subject when cells are expressing levels of IL-12 for example above 2,000 pg/10⁶ cells/ml/hr, optionally 2,500 pg/10⁶ cells/ml/hr, 5,000 pg/10⁶ cells/ml/hr, 7,500 pg/10⁶ cells/ml/hr, 10,000 pg/10⁶ cells/ml/hr. Other combinations are contemplated.

A further aspect includes a whole cell vaccine comprising an isolated cell or population of cells expressing and/or secreting IL-12 and one or more of IL-21 and IL-18 each above a threshold level, within a selected range and/or at a selected ratio, optionally comprising: a lentiviral vector; an IL-12 expression cassette; and an IL-21 expression cassette and/or an IL-18 expression cassette.

A further aspect includes a composition comprising an isolated virus, cell or population of cells comprising a lentiviral vector; an IL-12 expression cassette; and an IL-21 expression cassette and/or an IL-18 expression cassette.

A further aspect includes a method of expressing IL-12 and at least one of IL-21 and/or IL-18 in a cell, optionally a cancer cell, comprising contacting the cell with a composition, vector construct, or isolated virus comprising: a lentiviral vector; an IL-12 expression cassette; and an IL-21 expression cassette and/or an IL-18 expression cassette under conditions that permit transduction of the cell, thereby providing a transduced cell, optionally wherein the IL-12, IL-21, and/or IL-18 is secreted.

In an embodiment, the method further comprises a step of isolating the transduced cell or isolating a population of cells comprising the transduced cell.

In an embodiment, the method further comprises: growth arresting the transduced cell, the population of cells or composition; and introducing the transduced cell, population of cells and/or composition in a subject.

An aspect includes a method of reducing the number of tumor cells or cancer burden in a subject in need thereof comprising administering to the subject an isolated virus, transduced cell, population of cells, or composition comprising: a lentiviral vector; an IL-12 expression cassette; and an IL-21 expression cassette and/or an IL-18 expression cassette.

An aspect includes a method of treating a subject with cancer or an increased risk of cancer comprising administering to the subject an isolated virus, transduced cell, population of cells, or composition comprising: a lentiviral vector; an IL-12 expression cassette; and an IL-21 expression cassette and/or an IL-18 expression cassette.

In an embodiment, the method further comprises monitoring cancer progression.

In an embodiment, the cancer is leukemia, optionally ALL, AML, CML or CLL.

An aspect includes a method of inducing or enhancing an immune response in a subject comprising administering to the subject an isolated virus, transduced cell, population of cells, or composition comprising: a lentiviral vector; an IL-12 expression cassette; and an IL-21 expression cassette and/or an IL-18 expression cassette. In an embodiment, the subject has cancer or an increased risk of cancer.

An aspect includes a method of inducing or enhancing a memory immune response in a subject, comprising administering to the subject an isolated virus, transduced cell, population of cells, or composition comprising: a lentiviral vector; an IL-12 expression cassette; and an IL-21 expression cassette and/or an IL-18 expression cassette. In an embodiment the subject has cancer or an increased risk of cancer.

In an embodiment, the transduced cell is growth arrested prior to administering to the subject. In an embodiment, the transduced cell is irradiated prior to administering to the subject.

A further aspect includes a method of delivering IL-12 and at least one of IL-21 or IL-18 to a subject, comprising: generating an IL-12 and at least one of IL-21 or IL-18 secreting cell, optionally a cancer cell; and introducing an effective number of the generated IL-12 and at least one of IL-21 or IL-18 secreting cells to the subject. In an embodiment, the subject has cancer or an increased risk of cancer. In an embodiment, the method is for enhancing cancer treatment.

In an embodiment, the IL-12 and at least one of IL-21 or IL-18 secreting cell is generated by contacting the cell with a composition comprising a lentiviral delivery vector an IL-12 expression cassette and an IL-21 or IL-18 expression cassette.

In an embodiment, the cell is a cancer cell, optionally derived from the subject with cancer. In an embodiment, the cancer is a leukemia, optionally ALL, AML, CML or CLL.

In an embodiment, the IL-12 and at least one of IL-21 or IL-18 secreting cell is growth arrested prior to introducing to the subject.

In an embodiment, an immune response is initiated against a leukemia.

In an embodiment, the number of cells, preferably cancer cells, administered ranges from 10⁵ cells to 10⁹ cells, optionally about 10⁵, about 10⁶ cells, about 10⁷ cells, about 10⁸ cells, or about 10⁹ cells, optionally 10⁵ to 10⁶ cells, 10⁶ to 10⁷ cells, 10⁷ to 10⁸ cells, or 10⁸ to 10⁹ cells are administered. In an embodiment, the number of cells administered comprises at least 0.1×10⁶ IL-12 and at least one of IL-21 and/or IL-18 producing cells, optionally about 1×10⁶, about 2×10⁶, about 3×10⁶, about 4×10⁶, about 5×10⁶, about 6×10⁶, about 7×10⁶, about 8×10⁶, about 9×10⁶, about 10×10⁶, about 15×10⁶, about 20×10⁶, or more IL-12 and at least one of IL-21 and/or IL-18 producing cells, and wherein the population of cells secretes IL-12 and at least one of IL-21 and/or IL-18 levels necessary to induce or enhance a CD4+ T cell dependent immune response. In an embodiment the cancer is leukemia, lymphoma, myeloma, glioblastoma, melanoma, or cancer of the lung, ovary, prostate, breast, colon, bladder, liver, pancreas, thyroid, or head and neck.

A further aspect includes use of an isolated virus, transduced cell, population of cells, or composition comprising: a lentiviral vector; an IL-12 expression cassette; and an IL-21 expression cassette and/or an IL-18 expression cassette for reducing the number of tumor cells or cancer burden in a subject in need thereof.

A further aspect includes use of an isolated virus, transduced cell, population of cells, or composition comprising: a lentiviral vector; an IL-12 expression cassette; and an IL-21 expression cassette and/or an IL-18 expression cassette for treating a subject with cancer.

In an embodiment, the cancer is leukemia, optionally ALL, AML, CML or CLL.

An aspect includes use of an isolated virus, transduced cell, population of cells, or composition comprising: a lentiviral vector; an IL-12 expression cassette; and an IL-21 expression cassette and/or an IL-18 expression cassette for inducing or enhancing an immune response in a subject.

An aspect includes use of an isolated virus, transduced cell, population of cells, or composition comprising: a lentiviral vector; an IL-12 expression cassette; and an IL-21 expression cassette and/or an IL-18 expression cassette for inducing or enhancing a memory immune response in a subject.

In an embodiment, the transduced cell is growth arrested. In an embodiment, the transduced cell is irradiated.

An aspect includes use of an IL-12 and at least one of IL-21 or IL-18 secreting cell, optionally a cancer cell, for delivering IL-12 and at least one of IL-21 or IL-18 to a subject. In an embodiment, the subject has cancer or an increased risk of cancer optionally for enhancing cancer treatment: generating an IL-12 and at least one of IL-21 or IL-18 secreting cell; and obtaining or isolating the generated IL-12 and at least one of IL-21 or IL-18 secreting cells for introduction to the subject, wherein the secreting cells secrete IL-12 above a threshold and at least one of IL-21 or IL-18 above a threshold, within a range or at a selected ratio.

In an embodiment, the IL-12 and at least one of IL-21 or IL-18 secreting cell is generated by contacting the cell with a composition comprising a lentiviral delivery vector and an IL-12 expression cassette and an IL-21 or IL-18 expression cassette.

In an embodiment, the cell is optionally a leukemic cell, optionally derived from the subject with leukemia. In an embodiment the leukemia is ALL, AML, CML or CLL.

In an embodiment, the IL-12 and at least one of IL-21 or IL-18 secreting cell is growth arrested prior to introducing to the subject.

An aspect includes use of a composition, vector construct, virus, transduced cell, or population of cells comprising: a lentiviral vector; an IL-12 expression cassette; and an IL-21 expression cassette and/or an IL-18 expression cassette, for treating a subject with cancer such as leukemia or an increased risk of developing leukemia.

In an embodiment, the population of cells administered ranges from 10⁵ cells to 10⁹ cells, optionally about 10⁵ cells, about 10⁶ cells, about 10⁷, cells, about 10⁸ cells, or about 10⁹ cells.

In an embodiment, the population of cells administered comprises at least 0.1×10⁶ IL-12 and at least one of IL-21 and/or IL-18 producing cells, optionally about 1×10⁶, about 2×10⁶, about 3×10⁶, about 4×10⁶, about 5×10⁶, about 6×10⁶, about 7×10⁶, about 8×10⁶, about 9×10⁶, about 10×10⁶, about 15×10⁶, about 20×10⁶, or more IL-12 and at least one of IL-21 and/or IL-18 producing cells, and wherein the population of cells secretes IL-12 and at least one of IL-21 and/or IL-18 levels necessary to induce or enhance an immune response in a subject, for example a CD4+ T cell dependent immune response, a CD8 T cell response, an NK response, and/or a gamma delta T cell response.

The preceding section is provided by way of example only and is not intended to be limiting on the scope of the present disclosure and appended claims. Additional objects and advantages associated with the compositions and methods of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional advantages objects and embodiments are expressly included within the scope of the present disclosure. The publications and other materials used herein to illuminate the background of the disclosure, and in particular cases, to provide additional details respecting the practice, are incorporated by reference, and for convenience are listed in the appended reference section.

### DRAWINGS

Further objects, features and advantages of the disclosure will become apparent from the following detailed description taken in conjunction with the accompanying figures showing illustrative embodiments of the disclosure, in which:

FIG. 1 shows cooperation between IL-12 and IL-21 increasing the potency of leukemia cells transduced with Lentivirus (LV) vectors but producing a level of cytokine insufficient to provide full protection when injected alone at a ratio of 1:10. 1A) Four mice per group each injected IP with the following leukemia cells. Dotted line (circle): 10⁶ LV0 control. Solid line (square): 10⁶ LV12. Dashed line (triangle): mixture of 10⁶ LV0 and 10⁵ LV12. Shows some immunity but incomplete long term protection. This is typical of mice injected with leukemia clones secreting low amounts of IL-12. 1B) Four mice per group each injected IP with the following leukemia cells. Dotted line (circle): 10⁶ LV0 control. Solid line (square): 10⁶ LV21. Dashed line (triangle): mixture of 10⁶ LV0 and 10⁵ LV21. Shows some immunity but incomplete long term protection. This is typical of mice injected with leukemia clones secreting low amounts of IL-21. 1C) Four mice per group each injected IP with the following leukemia cells. Dotted line (circle): 10⁶ LV0 control. Solid line (square): 10⁶ double producer LV12+21. Dashed line (triangle): mixture of 10⁶ LV0 and 10⁵ double producer LV12+21. Shows enhanced immunity compared to either cytokine alone.

FIG. 2 shows cooperation between IL12 and IL18 increasing the potency of leukemia cells transduced with Lentivirus (LV) vectors but producing a level of cytokine insufficient to provide full protection when injected alone at a ratio of 1:10. 2A) Four mice per group each injected IP with the following leukemia cells. Dotted line (circle): 10⁶ LV0 control. Solid line (star): 10⁶ LV12. Dashed line (triangle): mixture of 10⁶ LV0 and 10⁵ LV12. Shows some immunity but incomplete long term protection. This is typical of mice injected with leukemia clones secreting low amounts of IL-12. 2B) Four mice per group each injected IP with the following leukemia cells. Dotted line (circle): 10⁶ LV0 control. Solid line (star): 10⁶ LV18. Dashed line (triangle): mixture of 10⁶ LV0 and 10⁵ LV18. Shows some immunity but incomplete long term protection. This is typical of mice injected with leukemia clones secreting low amounts of IL-18. 2C) Five mice per group each injected IP with the following leukemia cells. Dotted line (circle): 10⁶ LV0 control. Solid line (square): 10⁶ double producer LV12+18. Dashed line (triangle): mixture of 10⁶ LV0 and 10⁵ double producer LV12+18. Shows enhanced immunity compared to either cytokine alone.

FIG. 3 shows failure of IL-7 and IL-15 to cooperate with IL-12 in increasing the potency of leukemia cells transduced with Lentivirus (LV) vectors but producing a level of cytokine insufficient to provide full protection when injected alone at a ratio of 1:10. 3A) Four mice per group each injected IP with the following leukemia cells. Dotted line (circle): 10⁶ LV0 control. Solid line (square): 10⁶ LV12. Dashed line (triangle): mixture of 10⁶ LV0 and 10⁵ LV12. Shows some immunity but incomplete long term protection. This is typical of mice injected with leukemia clones secreting low amounts of IL-12. 3B) Four mice per group each injected IP with the following leukemia cells. Dotted line (circle): 10⁶ LV0 control. Solid line (square): 10⁶ double producer LV12+7. Dashed line (triangle): mixture of 10⁶ LV0 and 10⁵ double producer LV12+7. Shows IL-7 fails to enhance IL-12 response and actually diminishes ability of IL-12 to protect when 100% are secreting both IL-12 and IL7. 3C) Four mice per group each injected IP with the following leukemia cells. Dotted line (circle): 5×10⁴ LV0 control. Solid line (square): 5×10⁴ LV12 Dashed line (triangle): mixture of 5×10⁴ LV0 and 5×10³ LV12. Shows some immunity but incomplete long term protection. This is typical of mice injected with leukemia clones expressing low amounts of IL-12. 3D) Four mice per group each injected IP with the following leukemia cells. Dotted line (circle): 5×10⁴ LV0 control. Solid line (square): 5×10⁴ double producer LV12+15. Dashed line (triangle): mixture of 5×10⁴ LV0 and 5×10³ double producer LV12+15. Shows IL-15 fails to enhance IL-12 response.

FIG. 4 shows cooperation between IL12 and IL18 increasing the potency of leukemia cells transduced with Lentivirus (LV) vectors but producing a level of IL12 cytokine insufficient to provide full protection when co-injected with non-transduced cells at different ratios. 4A) Five mice per group each injected IP with the following ratios of non-transduced (LV0) leukemia cells and clone LV12, producing low amounts of IL12 (under 2,000 pg/ml/10⁶ cells/hr). Solid line (triangle): 10⁶ LV0:LV12 cells at a ratio of 50:50. Dotted line (inverted triangle): 10⁶ LV0:LV12 cells at a ratio of 90:10. Dashed line (triangle): 10⁶ LV0:LV12 at a ratio of 99:1. Shows some immunity at 50:50. 4B) Five mice per group each injected IP with the following ratios of non-transduced (LV0) leukemia cells and clone “LV12+18” producing low amounts of IL-12 (under 2,000 pg/hr/10⁶ cells)+low amounts of IL-18 (under 2,000 pg/ml/10⁶ cells/hr). Solid line (star): 10⁶ LV0:LV12+18 cells at a ratio of 50:50. Dotted line (star): 10⁶ LV0:LV12+18 cells at a ratio of 90:10. Dashed line (star): 10⁶ LV0:LV12+18 at a ratio of 99:1. Shows protection is achieved with fewer IL12+IL18 transduced cells compared to cells transduced with IL12 alone. In these experiments injection of 10⁶ LV0 alone leads to 0% survival before day 15 in all cases (data not shown). A total of 10⁶ LV cells were injected IV in all cases.

FIG. 5 shows co-expression of IL18 has little effect on the potency of leukemia cells producing higher levels of IL12 cytokine. 5A) Five mice per group each injected IP with the following ratios of non-transduced (LV0) leukemia cells and clone LV12, producing high amounts of IL12 (>10,000 pg/ml/10⁶ cells/hr). Solid line (star): 10⁶ LV0:LV12 cells at a ratio of 99:1. Dotted line (star): 10⁶ LV0:LV12 cells at a ratio of 999:1. 5B) Five mice per group each injected IP with the following ratios of non-transduced (LV0) leukemia cells and clone “LV12+18” producing high amounts of IL-12 (>10,000 pg/ml/10⁶ cells/hr)+high amounts of IL-18 (>5,000 pg/ml/10⁶ cells/hr). Solid line (triangle): 10⁶ LV0:LV12+18 cells at a ratio of 99:1. Dotted line (triangle): 10⁶ LV0:LV12+18 cells at a ratio of 999:1. Shows little difference in protection by co-expression of IL-18 using cells expressing >10,000 pg/ml/10⁶ cells/hr of IL-12. In these experiments injection of 10⁶ LV0 alone leads to 0% survival before day 15 in all cases (data not shown). A total of 10⁶ LV cells were injected IV in all cases.

### DETAILED DESCRIPTION

Clones of leukemic cells producing a wide range of IL-12 were established previously. Injection of IL-12 producing leukemic cells provoked long term and specific immunity without the induction of antagonistic mechanisms. The inventors previously found that injection of as few as 1% IL-12 producing leukemic cells along with 99% untransduced leukemic cells, was sufficient to elicit protective immunity as long as each of the transduced cells produced IL-12 above a threshold. The inventors have also previously shown that that mixtures containing small amounts of high IL-12 producing solid cancer cells lead to tumour clearance, whereas mixtures containing large amounts of low IL-12 producing cells fail to elicit protection, despite the production of equal amounts of total IL-12 in both mixtures (6).

The inventors show herein that injection of 10% of transduced leukemic cells producing low levels of IL-12 (e.g. below a threshold) along with untransduced leukemic cells elicits incomplete immunity. Similarly, the inventors found that injection of 10% transduced leukemic cells producing low levels of either IL-21 or IL-18 (e.g. below a threshold) along with untransduced leukemic cells elicits incomplete immunity. However, the injection of 10% of transduced leukemic cells producing the same low levels of IL-12 and low levels of either IL-21 or IL-18 along with untransduced leukemic cells was sufficient to elicit protective immunity. Leukemic cells producing a combination of low levels of IL-12 and low levels of either IL-15 or IL-7 did not elicit a similar effect.

Co-expression of IL-12 in combination with IL-21 and/or IL-18 may therefore increase clinical impact by improving the immune stimulating performance of the lower expressing cells, and provide protective immunity even when IL-12, IL-21 and IL-18 are secreted below the threshold level required to provide protective immunity individually. Coexpression of IL-12 and one or more of IL-21 and IL-18 lowers the effective IL-12 secretion level that provides immunity and can rescue IL-12 cells which secrete IL-12 below such threshold allowing them also to provide immunity. This may be important in therapeutic applications where cell transduction efficiencies are not robust enough to obtain a population of transduced cells expressing above the threshold level and/or where cells to be transduced are scarce.

The vector constructs, compositions, cells and methods described herein for delivering IL-12 in combination with either IL-21 or IL-18 are highly effective and are readily applied to a variety of cancers.

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure.

### Definitions

As used herein, the following terms may have meanings ascribed to them below, unless specified otherwise. However, it should be understood that other meanings that are known or understood by those having ordinary skill in the art are also possible, and within the scope of the present disclosure. All publications, patent applications, patents, and other references mentioned herein are incorporated by reference in their entirety. In the case of conflict, the present specification, including definitions, will control. In addition, the materials, methods, and examples are illustrative only and not intended to be limiting.

The term “a cell” as used herein includes a plurality of cells.

The term “ALL” as used herein refers to acute lymphoblastic leukemia is a rapidly growing leukemia wherein the malignant hematopoietic cells are lymphoid precursor cells. Cytogenetic abnormalities occur in ˜70% of cases of ALL in adults but are not associated with a single translocation event.

The term “allogenic” also referred to as “allogeneic” as used herein means cells, tissue, DNA, or factors taken or derived from a different subject of the same species. Allogenic tumor cells for use in methods for treating cancer are known in the art, and are reviewed for example in [131] and [132]. For example in the context where allogenic transduced cancer cells are administered to a subject with cancer, cancer cells removed from a patient that is not the subject, are transduced or transfected with a vector that directs the expression of IL-12 and one or more of IL-18 or IL-21 and the transduced cells are administered to the subject. The phrase “directs expression” refers to the polynucleotide comprising a sequence that encodes the molecule to be expressed. The polynucleotide may comprise additional sequence that enhances expression of the molecule in question.

The term “AML” as used herein refers to acute myeloid leukemia, a rapidly progressing disease in which too many immature non-lymphocyte white blood cells are present in the blood and bone marrow. Also called acute myelogenous leukemia, acute myeloblastic leukemia, acute nonlymphocytic leukemia, and ANLL.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.−16.6 (Log 10 [Na+])+0.41(%(G+C)−600/l), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5× sodium chloride/sodium citrate (SSC)/5×Denhardt's solution/1.0% SDS at Tm−5° C. based on the above equation, followed by a wash of 0.2×SSC/0.1% SDS at 60° C. Moderately stringent hybridization conditions include a washing step in 3×SSC at 42° C. It is understood, however, that equivalent stringencies may be achieved using alternative buffers, salts and temperatures. Additional guidance regarding hybridization conditions may be found in: Current Protocols in Molecular Biology, John Wiley & Sons, N.Y., 2002, and in: Sambrook et al., Molecular Cloning: a Laboratory Manual, Cold Spring Harbor Laboratory Press, 2001.

The term “autologous” as used herein refers to cells, tissue, DNA or factors taken or derived from an individual's own tissues, cells or DNA. For example in the context where autologous transduced cancer cells are administered to a subject with cancer, cancer cells removed from the subject are transduced or transfected with a vector that directs the expression of IL-12 and the transduced cells are administered to the subject.

The phrase “cancer burden” refers to the quantum of cancer cells or cancer volume in a subject. Reducing cancer burden accordingly refers to reducing the number of cancer cells or the cancer volume in a subject.

The phrase “cancer that is characterized by periods of remission” refer to cancers that may respond to a treatment but wherein the cancer recurs at some later time suggesting that not all cancer cells were eradicated by the treatment. An example of such a cancer is CLL.

The term “cassette” as used herein refers to a polynucleotide sequence that is to be expressed. The cassette can be inserted into a vector. The cassette optionally includes regulatory sequence to direct or modify its expression.

The term “CLL” refers to chronic lymphocytic leukemia, a slow growing type of leukemia. CLL is the most common leukemia of adults with an expectation of ˜16500 cases in North America in 2008. Remissions can be achieved with purine analogues and monoclonal antibody therapy however the diseases invariable progresses. CLL is also referred to as chronic lymphoblastic leukemia. B-CLL is a subset of CLL.

The term “clinical grade vector” as used herein refers to a vector manufactured using near-GMP or GMP procedures and quality assurance tested.

The term “CML” refers to chronic myeloid leukemia, a slowly progressing leukemia wherein excessive white blood cells are made in the bone marrow. The hallmark of this disease is the reciprocal translocation between chromosomes 9 and 22 leading to the formation of the Bcr-Abl oncogene. This is manifested by a rapid expansion of bone marrow-derived hematopoietic cells of the myeloid lineage. CML is also referred to as chronic myelogenous leukemia, and chronic granulocytic leukemia.

A “conservative amino acid substitution” as used herein, is one in which one amino acid residue is replaced with another amino acid residue without abolishing the protein's desired properties. Conservative amino acid substitutions are known in the art. For example, conservative substitutions include substituting an amino acid in one of the following groups for another amino acid in the same group: alanine (A), serine (S), and threonine (T); aspartic acid (D) and glutamic acid (E); asparagine (N) and glutamine (Q); arginine (R) and lysine (L); isoleucine (I), leucine (L), methionine (M), valine (V); and phenylalanine (F), tyrosine (Y), and tryptophan (W).

The term “detection cassette” as used herein refers to a polynucleotide that directs expression of a molecule that is useful for enriching, sorting, tracking and/or killing cells in which it is expressed. The detection cassette encodes a polypeptide that is expressed in the transduced or transfected cell and can as a result be used to detect and/or isolate transduced or transfected cells. The detection cassette is optionally used to determine the efficiency of cell transduction or transfection. For example, CD271, which is encoded by the lentiviral vectors described herein, may be used to determine the efficiency of cell transduction and/or isolate transduced cells.

As used herein, the phrase “effective amount” or “therapeutically effective amount” or a “sufficient amount” of composition, vector construct, virus or cell of the present application is a quantity sufficient to, when administered to the subject, including a mammal, for example a human, effect beneficial or desired results, including clinical results, and, as such, an “effective amount” or synonym thereto depends upon the context in which it is being applied. For example, in the context of treating cancer, it is an amount of the composition, vector construct, virus or cell sufficient to achieve a treatment response as compared to the response obtained without administration of the composition, vector construct, virus or cell. Also, as used herein, a “therapeutically effective amount” of a composition, vector construct, virus or cell of the present disclosure is an amount which results in a beneficial or desired result in a subject as compared to a control. Dosage regime may be adjusted to provide the optimum therapeutic response.

The term “hybridize” refers to the sequence specific non-covalent binding interaction with a complementary nucleic acid.

The term “immune response” as used herein can refer to activation of either or both the adaptive and innate immune system cells such that they shift from a dormant resting state to a state in which they are able to elaborate molecules typical of an active immune response.

The phrase “inducing an immune response” as used herein refers to a method whereby an immune response is activated. The phrase “enhancing an immune response” refers to augmenting an existing immune response.

The term “increased risk of cancer” as used herein means a subject that has a higher risk of developing a particular cancer than the average risk of the population. A subject may have a higher risk due to previously having had said particular cancer and or having a genetic risk factor for said particular cancer or exhibit a pre-cancer syndrome. For example, delivery of the constructs described herein to engineer IL-12 and one or more of IL-21 and IL-18 expression in dendritic cells or other efficient antigen-presenting cells could also be effective in a pre-cancerous state if dominant tumor-associated antigens had been identified and the host immune response re-directed against that antigen.

The term “kills” with respect to transfected or transduced cells refers to inducing cell death through any of a variety of mechanisms including apoptosis, necrosis and autophagy. For example an agent that is cytotoxic kills the cells.

The term “leukemia” as used herein refers to any cancer or precancerous syndrome that initiates in blood forming tissues such as the bone marrow. A number of leukemias have been characterized including ALL, AML, CLL, and CML.

The term “polynucleotide” and/or “nucleic acid sequence” as used herein refers to a sequence of nucleoside or nucleotide monomers consisting of naturally occurring bases, sugars and intersugar (backbone) linkages. The term also includes modified or substituted sequences comprising non-naturally occurring monomers or portions thereof. The nucleic acid sequences of the present application may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine.

The term “polypeptide” as used herein refers to a sequence of amino acids consisting of naturally occurring residues, and non-naturally occurring residues.

The term “promoter” as used herein refers to a recognition site on DNA that is bound by an RNA polymerase. For example, the polymerase drives transcription of the cassette or transgene downstream of the promoter. The promoter may be a constitutive promoter or an inducible promoter.

The term “sequence identity” as used herein refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present application. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). The percent identity between two sequences can be determined using techniques similar to those described above, with or without allowing gaps. In calculating percent identity, typically only exact matches are counted.

The term “subject” as used herein includes all members of the animal kingdom including mammals, suitably humans including patients.

The term “subject in need thereof” refers to a subject that could benefit from the method, treatment, or use, and optionally refers to a subject with cancer, such as leukemia, or optionally a subject with increased risk of cancer, such as a subject previously having cancer, a subject with a precancerous syndrome or a subject with a strong genetic disposition.

The term “transduction” as used herein refers to a method of introducing a vector construct or a part thereof into a cell. Wherein the vector construct is comprised in a virus such as for example a lentivirus, transduction refers to viral infection of the cell and subsequent transfer and integration of the vector construct or part thereof into the cell genome.

The term “treating” or “treatment” as used herein means administering to a subject a therapeutically effective amount of the compositions, cells or vector constructs of the present application and may consist of a single administration, or alternatively comprise a series of applications.

As used herein, and as well understood in the art, “treatment” or “treating” is also an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment. Further any of the treatment methods or uses described herein can be formulated alone or for contemporaneous administration with other agents or therapies.

The term “vector construct” as used herein means a recombinant polynucleotide comprising a vector alternatively referred to as a vector backbone and at least one coding cassette. A vector construct is optionally comprised in a virus, such as a lentivirus. The term “vector” as used herein refers to a means by which polynucleotides can be introduced into a cell or host.

Where a range of values is provided, it is understood that each intervening value, to the tenth of the unit of the lower limit unless the context clearly dictates otherwise, between the upper and lower limit of that range and any other stated or intervening value in that stated range is encompassed within the description. Ranges from any lower limit to any upper limit are contemplated. The upper and lower limits of these smaller ranges which may independently be included in the smaller ranges is also encompassed within the description, subject to any specifically excluded limit in the stated range. Where the stated range includes one or both of the limits, ranges excluding either both of those included limits are also included in the description.

As used herein and in the appended claims, the singular forms “a”, “an”, and “the” include plural references unless the context clearly dictates otherwise.

All numerical values are modified by “about” or “approximately” the indicated value, and take into account experimental error and variations that would be expected by a person having ordinary skill in the art.

The phrase “and/or” as used herein, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

All transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively

As used herein, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, in certain methods described herein that include more than one step or act, the order of the steps or acts of the method is not necessarily limited to the order in which the steps or acts of the method are recited unless the context indicates otherwise.

Any methods and materials similar or equivalent to those described herein can also be used in the practice or testing of the present disclosure.

### Vector Constructs and Virus

The application provides in one aspect a vector construct or virus such as a lentivirus comprising a delivery vector, an IL-12 expression cassette and one or more of an IL-21 or IL-18 expression cassette. In one embodiment the delivery vector is a lentiviral vector (LV) backbone.

### Interleukin-12 (IL-12) Expression Cassette

Interleukin-12 is a heterodimeric cytokine with multiple biological effects on the immune system. It is composed of two subunits, p35 and p40, both of which are required for the secretion of the active form of IL-12, p70. Interleukin-12 acts on dendritic cells (DC), leading to increased maturation and antigen presentation, which can allow for the initiation of a T cell response to tumor specific antigens.

In one embodiment the IL-12 expression cassette comprises a polynucleotide that directs expression of IL-12 polypeptide. Any IL-12 polypeptide including variants and derivatives of known IL-12 molecules can be used. In a preferred embodiment, the IL-12 is human IL-12. In another embodiment, the IL-12 is murine IL-12.

In one embodiment the polynucleotide comprises the sequence of both IL-12 subunits, p35 and p40, separated by an IRES sequence which permits expression of multiple transgenes from a single transcript. In other embodiments, the polynucleotide directs expression of an IL-12 fusion polypeptide that retains IL-12 activity. In one embodiment, the polynucleotide that directs the expression of IL-12 comprises a cDNA encoding a human IL-12 polypeptide fusion obtained from InVivoGen (pORF with IL-12elasti(p40::p35)).

The cDNA/nucleic acid can be codon optimized for efficient expression.

In one embodiment, the polynucleotide directs the expression of an IL-12 polypeptide comprising all or part of SEQ ID NO:4 or 5, and/or a variant of a fragment thereof that retains IL-12 activity. In another embodiment, the polynucleotide directs expression of an IL-12 fusion polypeptide that has at least 70%, 70-80%, 80-90%, 90-95%, 95-99.9% or more sequence identity to the IL-12 portion of SEQ ID NO:4 or 5 and retains IL-12 activity. IL-12 activity is determined for example by assessing activation of the IL-12 receptor in a cell based assay.

A person skilled in the art will understand that non-critical residues can be deleted, and or mutated without effect on IL-12. Polynucleotides directing expression of IL-12 polypeptide analogs are also contemplated.

In various embodiments, the IL-12 polypeptide/IL-12 expression cassette comprises a signal sequence, optionally endogenous or exogenous, for example a Immunoglobulin kappa (Igkappa) signal sequence or a IL-2 signal sequence, preferably a human signal sequence such as human IL-2 signal sequence

### Interleukin-21 (IL-21) Expression Cassette

Interleukin-21 is a 17 kDa type 1 four-α-helical bundle cytokine primarily produced by Natural Killer T (NKT) cells and T cells.

The IL-21 expression cassette comprises a polynucleotide that directs expression of IL-21 polypeptide. Any IL-21 polypeptide including variants and derivatives of known IL-21 molecules can be used. In a preferred embodiment, the IL-21 is human IL-21. In another embodiment, the IL-21 is murine IL-21.

In one embodiment, the polynucleotide that directs the expression of IL-21 comprises a cDNA/nucleic acid encoding a human IL-21 polypeptide.

The IL-21 polypeptide sequence is optionally human IL-21 Uniprot accession number Q9HBE4 and the nucleotide sequence is optionally human IL-21 accession number HGNC:6005.

The cDNA/nucleic acid can be codon optimized for efficient expression.

In one embodiment, the polynucleotide directs the expression of an IL-21 polypeptide comprising all or part of SEQ ID NO: 7, and/or a variant of a fragment thereof that retains IL-21 activity. IL-21 activity is determined for example by assessing activation of the IL-21 receptor in a cell based assay.

A person skilled in the art will understand that non-critical residues can be deleted, and or mutated without effect on IL-21. Polynucleotides directing expression of IL-21 polypeptide analogs are also contemplated.

In various embodiments, the IL-21 polypeptide/IL-21 expression cassette comprises a signal sequence, optionally endogenous or exogenous, for example a Immunoglobulin kappa (Igkappa) signal sequence or a IL-2 signal sequence, preferably a human signal sequence such as human IL-2 signal sequence.

### Interleukin-18 (IL-18) Expression Cassette

Interleukin-18 is a member of the IL-1 superfamily.³³⁻³⁵

The IL-18 expression cassette comprises a polynucleotide that directs expression of IL-18 polypeptide. Any IL-18 polypeptide including variants and derivatives of known IL-18 molecules can be used. In a preferred embodiment, the IL-18 is human IL-18. In another embodiment, the IL-18 is murine IL-18.

The IL-18 polypeptide/IL-18 expression cassette comprises a signal sequence, for example a Immunoglobulin kappa (Igkappa) signal sequence or a IL-2 signal sequence, preferably a human signal sequence such as human IL-2 signal sequence. As shown herein, the inventors found the signal sequence from human IL-2 produces a higher level of IL-18 expression. Accordingly, in an embodiment, the signal sequence is a human IL-2 signal sequence.

In one embodiment, the polynucleotide that directs the expression of IL-18 comprises a cDNA encoding a human IL-18 polypeptide. The IL-18 polypeptide sequence is optionally human IL-18 Uniprot accession number Q14116 or the IL-18 nucleotide sequence is optionally human IL-18 accession number HGNC:5986.

The cDNA/nucleic acid can be codon optimized for efficient expression.

In one embodiment, the polynucleotide directs the expression of an IL-18 polypeptide comprising all or part of SEQ ID NO: 6, and/or a variant of a fragment thereof that retains IL-18 activity. IL-18 activity is determined for example by assessing activation of the IL-18 receptor in a cell based assay.

A person skilled in the art will understand that non-critical residues can be deleted, and or mutated without effect on IL-18. Polynucleotides directing expression of IL-18 polypeptide analogs are also contemplated.

### Expression Cassettes for Simultaneous Expression of IL-12 and One or More of IL-21 or IL-18

The skilled person will be familiar with techniques that engineer the expression of multiple genes from a single construct, including single promoter constructs where an internal ribosome entry site (IRES) element is placed before the second gene; single promoter constructs containing sequences that encode “self-cleaving” 2A peptides between genes; and dual promoter constructs.¹³⁸⁻¹⁴¹ A bicistronic construct can be created using any suitable backbone to combine the fused IL-12 construct (encoding both p40 and p35) with an IL-21 or an IL-18 construct inserted downstream of an IRES site. In an alternative embodiment, the positions of the IL-12 and IL-21 or IL-18 constructs relative to the IRES can be reversed. In a further embodiment, the IL-12 and one or more of the IL-21 or IL-18 constructs can be separated by a self-cleaving 2A sequence such as for example P2A. Other 2A sites may also be used. The skilled person will appreciate that the selection of one or more promoters, IRES, and/or 2A sequence can be varied to achieve the desired expression levels. For example, use of a 2A peptide is expected to yield higher expression levels compared to other methods.⁶¹

The IL-12 encoding polynucleotide, and one or more of the IL-21 expressing polynucleotide and the IL-18 expressing polynucleotide can be comprised in a vector construct, separated for example by an IRES sequence which permits expression of multiple transgenes from a single transcript.

For example, in some embodiments, the IL-12, and one or more of the IL-21 and IL-18 cassettes are fused.

In one embodiment, the IL-21 expression cassette and the IL-12 expression cassette are fused e.g. form an IL-12-IL-21 expression cassette or IL-21-IL-12 expression cassette wherein the IL-12 and IL-21 encoding cDNAs/nucleic acids are separated by an IRES sequence or 2A peptide encoding sequence which permits the expression of IL-12 and IL-21 from a single transcript. In such expression cassettes, a single promoter can direct expression of the transcript which is then translated to produce IL-12 and IL-21.

In one embodiment, the IL-18 expression cassette and the IL-12 expression cassette form an IL-12-IL-18 expression cassette or IL-18-IL-12 expression cassette wherein the IL-12 and IL-18 encoding cDNAs/nucleic acids are separated by an IRES sequence or 2A peptide encoding sequence which permits the expression of IL-12 and IL-18 from a single transcript. In such an expression cassette, a single promoter directs expression of the transcript which is then translated to produce IL-12 and IL-18.

In some embodiments, the IL-12, and one or more of the IL-21 and IL-18 cassettes are not fused. For example, expression of the IL-12 cassette may be directed by a first promoter, and expression of the one or more of the IL-21 and IL-18 cassettes may be directed by a second promoter. In an embodiment, the first promoter is a constitutive promoter and the second promoter is an inducible promoter. In an embodiment, the first promoter is an inducible promoter and the second promoter is a constitutive promoter.

Embodiments relating to IL-12 expression cassette are applicable to when the IL-12 expression cassette is monocistronic or multicistronic. Similarly embodiments relating to IL-21 or IL-18 expression cassettes are applicable to when IL-21 or IL-18 expression cassettes respectively are monocistronic or multicistronic.

The vector construct can be designed and/or cells prepared where the level of the secreted cytokines is similar or dissimilar as described further below.

### Delivery Vectors

A variety of delivery vectors and expression vehicles can be usefully employed to introduce a modified DNA molecule into a cell. Vectors that are useful comprise lentiviruses, oncoretroviruses, expression plasmids, adenovirus, and adeno-associated virus. Other delivery vectors that are useful comprise herpes simplex viruses, transposons, vaccinia viruses, human papilloma virus, Simian immunodeficiency viruses, HTLV, human foamy virus and variants thereof. Further vectors that are useful comprise spumaviruses, mammalian type B retroviruses, mammalian type C retroviruses, avian type C retroviruses, mammalian type D retroviruses, HTLV/BLV type retroviruses, and lentiviruses.

Vectors such as those listed above have been employed to introduce DNA molecules into cells for use in gene therapy. Examples of vectors used to express DNA in cells include vectors described in: Kanazawa T, Mizukami H, Okada T, Hanazono Y, Kume A, Nishino H, Takeuchi K, Kitamura K, Ichimura K, Ozawa K. Suicide gene therapy using AAV-HSVtk/ganciclovir in combination with irradiation results in regression of human head and neck cancer xenografts in nude mice. Gene Ther. 2003 January; 10(1):51-8. Fukui T, Hayashi Y, Kagami H, Yamamoto N, Fukuhara H, Tohnai I, Ueda M, Mizuno M, Yoshida J Suicide gene therapy for human oral squamous cell carcinoma cell lines with adeno-associated virus vector. Oral Oncol. 2001 Apr.; 37(3):211-5.

### Retroviral Vectors

In one embodiment, the delivery vector is a retroviral vector. In a further embodiment, the delivery vector is a lentiviral vector. Lentiviral vectors (LVs), a subset of retroviruses, transduce a wide range of dividing and non-dividing cell types with high efficiency, conferring stable, long-term expression of the transgene²⁵⁻²⁷.

The use of lentivirus-based gene transfer techniques relies on the in vitro production of recombinant lentiviral particles carrying a highly deleted viral genome in which the transgene of interest is accommodated. In particular, the recombinant lentivirus are recovered through the in trans coexpression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral cDNA deprived of all open reading frames, but maintaining the sequences required for replication, incapsidation, and expression, in which the sequences to be expressed are inserted.

In one embodiment the lentiviral vector comprises one or more of a 5′-Long terminal repeat (LTR), HIV signal sequence, HIV Psi signal 5′-splice site (SD), delta-GAG element, Rev Responsive Element (RRE), 3′-splice site (SA), Elongation factor (EF) 1-alpha promoter and 3′-Self inactivating LTR (SIN-LTR). The lentiviral vector optionally comprises a central polypurine tract (cPPT; SEQ ID NO: 2) and a woodchuck hepatitis virus post-transcriptional regulatory element (WPRE; SEQ ID NO: 3).

In a further embodiment, the lentiviral vector comprises a pHR′ backbone or a 3′SIN, HIV-1-based, lentiviral backbone pDY.cPPT-EF1α.WPRE (144). In certain embodiments, the pHR′ back bone comprises for example as provided below.

In one embodiment the Lentigen lentiviral vector described in Lu, X. et al. Journal of gene medicine (2004) 6:963-973 is used to express the DNA molecules and/or transduce cells.

In one embodiment the lentiviral vector comprises a 5′-Long terminal repeat (LTR), HIV signal sequence, HIV Psi signal 5′-splice site (SD), delta-GAG element, Rev Responsive Element (RRE), 3′-splice site (SA), Elongation factor (EF) 1-alpha promoter and 3′-Self inactivating LTR (SIN-LTR). It will be readily apparent to one skilled in the art that optionally one or more of these regions is substituted with another region performing a similar function.

One or more regulatory elements can be included to direct differential or similar levels of expression of the cytokines. In certain embodiments the IL-12 and one or more of IL-21 or IL-18 is required to be expressed above a threshold level, in a particular range and/or at a certain ratio. For example, the ratio of IL-12 to IL-21 or IL 12 to IL-18 may be about 1:1, 2:1, 5:1, 10:1 or any other suitable ratio. Suitable ratios can be obtained for example by selecting a suitable promoter sequence for the expression cassette. In the case of constructs comprising an IRES sequence, ratios can be varied for example by reversing the relative position of the IL-12 and IL-21 or IL-12 and IL-18 relative to the IRES.

Transgene expression is driven by a promoter sequence. Optionally, the lentiviral vector comprises a CMV promoter. In another embodiment, the promoter is Elongation factor (EF) 1-alpha promoter. A person skilled in the art will be familiar with a number of promoters that will be suitable in the vector constructs described herein. In the case of dual-promoter constructs, different promoters can be selected that provide the desired level of expression for each expression cassette. For example, expression of the IL-12 cassette may be directed by a first promoter, and expression of the one or more of the IL-21 and IL-18 cassettes may be directed by a second promoter. One or both of the first and second promoters may independently be a constitutive promoter and/or an inducible promoter. Suitable constitutive promoters may include, without limitation, human Ubiquitin C (UBC), human Elongation Factor 1alpha (EF1A), human phosphoglycerate kinase 1 (PGK), simian virus 40 early promoter (SV40), and cytomegalovirus immediate-early promoter (CMV). Suitable inducible promoters may include, without limitation, an RTS gene switch, described for example in [145], and a tetracycline response element (TRE) (e.g. Tet-ON or Tet-OFF systems), described for example in [133] and [134].

Enhancer elements can be used to increase expression of modified DNA molecules or increase the lentiviral integration efficiency. In one embodiment the lentiviral vector further comprises a nef sequence. In a preferred embodiment the lentiviral further comprises a cPPT sequence which enhances vector integration. The cPPT acts as a second origin of the (+)-strand DNA synthesis and introduces a partial strand overlap in the middle of its native HIV genome. The introduction of the cPPT sequence in the transfer vector backbone strongly increased the nuclear transport and the total amount of genome integrated into the DNA of target cells. In an alternate preferred embodiment, the lentiviral vector further comprises a Woodchuck Posttranscriptional Regulatory Element (WPRE). The WPRE acts at the transcriptional level, by promoting nuclear export of transcripts and/or by increasing the efficiency of polyadenylation of the nascent transcript, thus increasing the total amount of mRNA in the cells. The addition of the WPRE to lentiviral vector results in a substantial improvement in the level of transgene expression from several different promoters, both in vitro and in vivo. In a further preferred embodiment, the lentiviral vector comprises both a cPPT sequence and WPRE sequence. In yet a further embodiment, the lentiviral vector comprises a sequence having at least 70%, 70-80%, 80-90%, 90-95%, 95-99.9% or more sequence identity to SEQ ID NO:2 and/or SEQ ID NO:3. The vector also comprises in an alternate embodiment an internal ribosome entry site (IRES) sequence that permits the expression of multiple polypeptides from a single promoter.

In addition to IRES sequences, other elements which permit expression of multiple polypeptides are useful. In one embodiment the vector comprises multiple promoters that permit expression more than one polypeptide. In another embodiment the vector comprises a protein cleavage site that allows expression of more than one polypeptide. Examples of protein cleavage sites that allow expression of more than one polypeptide comprise those listed in the following articles which are incorporated by reference: Retroviral vector-mediated expression of HoxB4 in hematopoietic cells using a novel coexpression strategy. Klump H, Schiedlmeier B, Vogt B, Ryan M, Ostertag W, Baum C. Gene Ther. 200; 8(10):811-7; A picornaviral 2A-like sequence-based tricistronic vector allowing for high-level therapeutic gene expression coupled to a dual-reporter system Mark J. Osborn, Angela Panoskaltsis-Mortari, Ron T. McElmurry, Scott K. Bell, Dario A. A. Vignali, Martin D. Ryan, Andrew C. Wilber, R. Scott Mclvor, Jakub Tolar and Bruce R. Blazar. Molecular Therapy 2005; 12 (3), 569-574; Development of 2A peptide-based strategies in the design of multicistronic vectors. Szymczak A L, Vignali D A. Expert Opin Biol Ther. 2005; 5(5):627-38; Correction of multi-gene deficiency in vivo using a single ‘self-cleaving’ 2A peptide-based retroviral vector. Szymczak A L, Workman C J, Wang Y, Vignali K M, Dilioglou S, Vanin E F, Vignali D A. Nat Biotechnol. 2004; 22(5):589-94. Other elements that permit expression of multiple polypeptides may be utilized in the vectors of the invention.

In various embodiments, signal sequences are included for directing secretion of one or more polypeptides. The signal sequence can for example be an antibody signal sequence, optionally an IgK signal sequence or a signal sequence from a secreted protein such as IL-2. Preferably the signal sequence is human. The signal sequence is operatively connected to the sequence to be secreted (e.g. fused thereto).

In certain embodiments, the lentiviral vector is a clinical grade vector.

### Viral Regulatory Elements

The viral regulatory elements are components of delivery vehicles used to introduce nucleic acid molecules into a host cell. The viral regulatory elements are optionally retroviral regulatory elements. For example, the viral regulatory elements may be the LTR and gag sequences from HSC1 or MSCV. The retroviral regulatory elements may be from lentiviruses or they may be heterologous sequences identified from other genomic regions.

### Safety Components

### Activator Polynucleotides

A number of safety components that can be introduced into the vector constructs disclosed are described in U.S. application Ser. No. 11/559,757, THYMIDYLATE KINASE MUTANTS AND USES THEREOF and U.S. application Ser. No. 12/052,565 which are incorporated herein by reference. In one embodiment, the lentiviral construct further comprises an activator polynucleotide encoding a polypeptide that converts a prodrug to a drug, optionally a modified tmpk polynucleotide. In one embodiment, the activator polynucleotide encodes a tmpk polypeptide, for example as disclosed in in U.S. application Ser. No. 11/559,757, and U.S. application Ser. No. 12/052,565. Other suitable cell fate components can be used.

The safety facet of cell fate control relies on efficient delivery and stable, consistent expression of both the therapeutic and the safety component genes.

### Expression Cassette Variants and Analogs

In the context of a polypeptide, the term “analog” as used herein includes any polypeptide having an amino acid residue sequence substantially identical to any of the wild type polypeptides expressed by the expression cassette that retains immune modulatory function of the wild-type polypeptide, for example at least 80% activity. For example, an analog of IL-12, is one in which one or more residues have been added, removed, or substituted, optionally conservatively substituted with a functionally similar residue, and which displays the ability to activate the IL-12 receptor similar to wild-type IL-12. Similarly, an analog of IL-21, is one in which one or more residues have been added, removed, or substituted, optionally conservatively substituted with a functionally similar residue, and which displays the ability to activate the IL-21 receptor similar to wild-type IL-21 and an analog of IL-18, is one in which one or more residues have been added, removed, or substituted, optionally conservatively substituted with a functionally similar residue, and which displays the ability to activate the IL-18 receptor similar to wild-type IL-18. Examples of conservative substitutions include the substitution of one non-polar (hydrophobic) residue such as alanine, isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another. The phrase “conservative substitution” also includes the use of a chemically derivatized residue in place of a non-derivatized residue provided that such polypeptide displays the requisite activity.

In the context of a polypeptide, the term “derivative” as used herein refers to a polypeptide having one or more residues chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as derivatives are those peptides which contain one or more naturally occurring amino acid derivatives of the twenty standard amino acids. For examples: 4-hydroxyproline may be substituted for proline; 5 hydroxylysine may be substituted for lysine; 3-methylhistidine may be substituted for histidine; homoserine may be substituted for serine; and ornithine may be substituted for lysine. Polypeptides of the present invention also include any polypeptide having one or more additions and/or deletions of residues relative to the wild type sequence, so long as the requisite activity is maintained.

The methods of making recombinant proteins are well known in the art and are also described herein.

The nucleic acids described herein can also comprise nucleotide analogs that may be better suited as therapeutic or experimental reagents. The nucleic acid can also contain groups such as reporter groups, a group for improving the pharmacokinetic properties of an nucleic acid.

The nucleic acid molecules may be constructed using chemical synthesis and enzymatic ligation reactions using procedures known in the art. The nucleic acid molecules of the invention or a fragment thereof, may be chemically synthesized using naturally occurring nucleotides or variously modified nucleotides designed to increase the biological stability of the molecules.

### Isolated Virus

The retroviral and lentiviral constructs are in one embodiment, packaged into viral particles. Methods for preparing virus are known in the art and described herein. In one embodiment, the application provides an isolated virus, optionally a lentivirus comprising the vector construct.

Methods of isolating virus are also known in the art and further described herein.

Methods of Expressing IL-12 and one or more of IL-21 or IL-18 in Cells and Cell Isolation

In one aspect, methods for expressing IL-12 and one or more of IL-21 or IL-18 in cells above a threshold level, within a selected range or at a selected ratio are provided. For example, the threshold level, range and/or ratio can be determined by identifying the level of expression that for the cytokine of interest (e.g. IL-12) produces an incomplete immunity when administered to mice at a 1:10 ratio with untransduced cells but produces complete immunity when administered at 10% when further expressing either IL-21 or IL-18 as described in the examples.

Accordingly, in one aspect, the application provides a method of expressing IL-12 and one or more of IL-21 or IL-18 in a cell above a threshold level, within a selected range and/or at a selected ratio.

The polynucleotides may be incorporated into an appropriate expression vector which ensures good expression of the IL-12, IL-21, IL-18, and/or other expression cassettes herein described. For example, vectors described herein are suitable.

Possible expression vectors include but are not limited to cosmids, plasmids, or modified viruses (e.g. replication defective retroviruses, adenoviruses and adeno-associated viruses), so long as the vector is compatible with the host cell used. The expression vectors are “suitable for transformation of a host cell”, which means that the expression vectors contain a nucleic acid molecule and regulatory sequences selected on the basis of the host cells to be used for expression, which is operatively linked to the nucleic acid molecule. Operatively linked or operably linked is intended to mean that the nucleic acid is linked to regulatory sequences in a manner which allows expression of the nucleic acid.

The application therefore includes a recombinant expression vector containing a nucleic acid molecule disclosed herein, or a fragment thereof, and the necessary regulatory sequences for the transcription and translation of the inserted protein-sequence.

Suitable regulatory sequences may be derived from a variety of sources, including bacterial, fungal, viral, mammalian, or insect genes (For example, see the regulatory sequences described in Goeddel, Gene Expression Technology: Methods in Enzymology 185, Academic Press, San Diego, Calif. (1990)). Selection of appropriate regulatory sequences is dependent on the host cell chosen as discussed below, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector.

Recombinant expression vectors can be introduced into host cells to produce a transformed host cell. The terms “transformed with”, “transfected with”, “transformation” “transduced” and “transfection” are intended to encompass introduction of nucleic acid (e.g. a vector or vector construct) into a cell by one of many possible techniques known in the art. The phrase “under suitable conditions that permit transduction or transfection of the cell” refers to for example for ex vivo culture conditions, such as selecting an appropriate medium, agent concentrations and contact time lengths which are suitable for transfecting or transducing the particular host. Suitable conditions are known in the art and/or described herein. The term “transformed host cell” or “transduced host cell” as used herein is intended to also include cells capable of glycosylation, for example mammalian and in particular human cells, that have been transformed with a recombinant vector or expression cassette disclosed herein. For example, nucleic acid can be introduced into mammalian cells via conventional techniques such as calcium phosphate or calcium chloride co-precipitation, DEAE-dextran mediated transfection, lipofectin, electroporation or microinjection. Suitable methods for transforming and transfecting host cells can be found in Sambrook et al. (Molecular Cloning: A Laboratory Manual, 3rd Edition, Cold Spring Harbor Laboratory Press, 2001), and other laboratory textbooks. Suitable methods for transducing cells are known in the art and are also described herein.

Vector constructs are introduced into cells that are used for transplant or introduced directly in vivo in mammals, preferably a human. The vector constructs are typically introduced into cells ex vivo using methods known in the art. Methods for introducing vector constructs comprise transfection, infection, electroporation. These methods optionally employ liposomes or liposome like compounds. Introduction in vivo optionally includes intravenous injection and/or intratumoral injection. These methods are described more fully elsewhere.

As shown in the Examples, the expression cassettes encoding the IL-12 and one or more of IL-21 or IL-18 may be incorporated into separate expression vectors, and may introduced into the cell simultaneously or sequentially. The IL-12 and one or more of IL-21 or IL-18 may be introduced into the cell in any order. For example, an IL-12 expression cassette may be introduced into the cell, and then an IL-21 or IL-18 expression cassette may be introduced into the cell. Alternately, an IL-21 or IL-18 expression cassette may be introduced into the cell and then an IL-12 expression cassette may be introduced into the cell. Accordingly, in one embodiment, the method of expressing IL-12 and one or more of IL-21 and IL-18 in a cell comprises contacting the cell with a composition, vector construct, and/or isolated virus comprising an IL-12 expression cassette under conditions that permit transduction or transfection of the cell to obtain an IL-12 expressing cell, and contacting the IL-12 expressing cell with a composition, vector construct, and/or isolated virus comprising one or more of an IL-21 or IL-18 expression cassette under conditions that permit transduction or transfection of the cell to obtain an IL-12 and one or more of IL-21 or IL-18 expressing cell. In another embodiment, the method of expressing IL-12 and one or more of IL-21 and IL-18 in a cell comprises contacting the cell with a composition, vector construct, and/or isolated virus comprising one or more of an IL-21 or IL-18 expression cassette under conditions that permit transduction or transfection of the cell to obtain an IL-21 or IL-18 expressing cell, and contacting the IL-21 or IL-18 expressing cell with a composition, vector construct, optionally one described herein, and/or isolated virus comprising an IL-12 expression cassette under conditions that permit transduction or transfection of the cell to obtain an IL-12 and one or more of IL-21 or IL-18 expressing cell.

In certain embodiments, the expression cassettes encoding IL-12 and one or more of IL-21 or IL-18 are incorporated into a single expression vector, such as a lentiviral vector. In other embodiments, they are in separate expression vectors. Accordingly, in certain embodiments, the cell is contacted with a composition, vector construct and/or isolated virus described herein, for example an isolated virus comprising a lentiviral vector backbone, an IL-12 expression cassette, and one or more of an IL-21 or IL-18 expression cassette or a combined IL-12-IL-21 (in either orientation) or a combined IL-12-IL-18 expression cassette in either orientation, under conditions that permit transduction or transfection of the cell. Methods of transducing cells are well known in the art. As used herein, reference to an IL-12 expression cassette and IL-21 expression cassette includes reference to two separate cassettes each with the elements necessary for directing expression or a combined expression cassette comprising for example and IRES and producing a single transcript.

In one embodiment, the method of expressing IL-12, and one or more of IL-21 and IL-18 in a cell comprises contacting the cell with a composition and/or vector construct described herein, for example comprising a lentiviral vector, an IL-12 expression cassette, and one or more of an IL-21 or IL-18 expression cassette, under conditions that permit transduction or transfection of the cell.

In other embodiments, the cells are optionally transduced with retroviral constructs that drive expression of IL-12 and one or both of IL-21 or IL-18, and/or additional expression cassettes described herein. Methods of transducing cells are well known in the art. Methods of transducing cells with lentiviral vectors are also described herein.

In certain embodiments, for example where the expression of one or more of the IL-12, and/or IL-21, and/or IL-18 cassettes are driven by an inducible promoter, the method further comprises contacting the cells with a suitable inducing agent, for example in the case of an RTS gene switch, the inducing agent may be VDX, or in the case of a TRE system, the inducing agent may be tetracycline.

In another embodiment, the method further comprises isolating the transduced cell or a population of transduced cells.

In one embodiment cells are isolated from the transduction or transfection medium and/or the viral preparation. For example the cells may be spun down and/or washed with a buffered saline solution. Accordingly, the cells can comprise a population of cells comprising transduced and untransduced cells. The inventors have been able to achieve up to 85% transduction efficiency. Accordingly, in certain embodiments, the population of cells comprises at least 1%, 2-5%, 5-10%, 10-15%, 15-20%, 20-25%, 25-30%, 30-40%, 40-50%, 50-60%, 60-70%, 70-80%, or up to 85% or more IL-12 and one or more of IL-21 or IL-18 transduced or transfected cells.

After transduction or transfection with vector constructs comprising an IL-12 expression cassette, and one or both of an IL-21 or IL-18 expression cassette, cells expressing these molecules are optionally isolated or enriched by a variety of means known in the art. In some embodiments, the IL-12 and at least one of IL-21 or IL-18 expressing cells are isolated or enriched using cytokine capture techniques. Cell surface or other markers provided by the expression vector can also be used to isolate of capture cytokine expressing cells. For example, CD271 is encoded in the lentiviral vectors described herein and can be used to select for lentiviral-transduced cells. Accordingly, in some embodiments, the IL-12 and at least one of IL-21 or IL-18 expressing cells are isolated or enriched using a detectable marker, optionally for cells expressing CD271. In some embodiments, the cells are enriched using magnetic cell sorting techniques (e.g. MACS from Meltenyi Biotec) using beads that are linked to an antibody to the target of interest eg CD271. Since the vector would encode both IL-12 and the second cytokine as well as CD271 the cells that are enriched would express both cytokines. In another embodiment, cells are enriched using a cell sorter.

The various selection and enrichment methods can be used to obtain a population of cells that are enriched for transduced or transfected cells, for example the population of cells can comprise at least 50%, at least 60%, at least 70%, at least 80%, at least 90%, at least 95%, at least 99%, or more than 99% IL-12 and one or more of IL-21 or IL-18 transduced or transfected cells.

Provided herein is in one aspect a whole cell vaccine comprising the transduced/transfected cells or populations of cells described herein, suitably formulated for human administration. For example, transduced/transfected cells can be resuspended in infusion buffer comprising, for example, Plasma-Lyte® A plus 0.5% human serum and/or human serum albumin. In an embodiment, the whole cell vaccine is an autologous whole cell vaccine. In an embodiment, the whole cell vaccine is an allogenic whole cell vaccine.

Cells expressing polynucleotides of the invention are, in an alternate embodiment, isolated using magnetic sorting. Additionally, cells may be isolated by drug selection. In one embodiment, a vector comprising a drug resistance gene and a polynucleotides of the invention is introduced into cells. Examples of drug resistance genes include, but are not limited to, neomycin resistance gene, blasticidin resistance gene (Bsr), hygromycin resistance gene (Hph), puromycin resistance gene (Pac), Zeocin resistance gene (Sh ble), FHT, bleomycin resistance gene and ampicillin resistance gene. After transduction or transfection, modified cells including the drug resistance gene are selected by adding the drug that is inactivated by the drug resistance gene. Cells expressing the drug resistance gene survive while non-transfected or non-transduced cells are killed. A person skilled in the art would be familiar with the methods and reagents required to isolate cells expressing the desired polynucleotides.

In a further embodiment, the transduced cells are growth arrested. Several methods can be used to growth arrest cells. In one embodiment, the transfected or transduced cells are growth arrested by irradiation. The term “growth arrested” refers to being inhibited for cell division. A person skilled in the art would recognize that the suitable irradiation dose to growth arrest a cell or population of cells may vary upon the cell type and/or number of cells. In one embodiment, the dose is about 75-150G. In another embodiment, for AML the dose of radiation is about 75G.

### Host Cells

The disclosure also provides in one aspect a cell (including for example an isolated cell in vitro, a cell in vivo, or a cell treated ex vivo and returned to an in vivo site) expressing and/or secreting IL-12 and one or more of IL-21 and IL-18 each above a threshold level, within a selected range and/or at a selected ratio. In one embodiment, the cell is transduced with a vector construct, virus or composition described herein.

Cells transfected with a nucleic acid molecule such as a DNA molecule, or transduced with the nucleic acid molecule such as a DNA or RNA virus vector, are optionally used, for example, in bone marrow or cord blood cell transplants according to techniques known in the art.

Any suitable cell may be used for transduction with the vector constructs described herein to obtain a cell secreting IL-12 and one or more of IL-21 and IL-18 each above a threshold level, within a selected range and/or at a selected ratio. In one embodiment, the cell is a cancer cell. In one embodiment, the cancer cell is a primary cancer cell. In a further embodiment, the primary cancer cell is derived from a subject. The cancer cell is optionally an allogenic or autologous cell. The cancer cell to be transduced is optionally derived from, propagated from or cloned from a cancer cell obtained from a subject. The cancer cell is in one embodiment obtained from the subject by biopsy. Alternatively, the cancer cell can be obtained from a blood sample, for example in the case of a leukemia, where the disease cell type is present in the peripheral blood. Methods for isolating cancer cells from a blood sample are known in the art and/or described herein.

Any cancer cell that can be transduced or transfected is a suitable host for transduction or transfection using a composition or vector construct of the application. In one embodiment the cancer cell is a leukemia cell. In one embodiment the leukemia cell is an acute lymphoblastic leukemia (ALL) cell, a chronic lymphoblastic leukemia (CLL) cell, chronic myeloid leukemia (CML) cell, or acute myeloid leukemia (AML) cell. In certain embodiments, the cancer cell is derived from a cancer that is characterized by or can exhibit periods of remission. In certain embodiments, the cancer cell is a metastatic cancer cell. In other embodiments, the cancer cell is a lymphoma, myeloma, glioblastoma, melanoma, tumor of the lung, ovary, prostate, breast, colon, bladder, liver, pancreas, thyroid, head or neck cancer cell. The immune system is able to seek out cells residing in nearly all parts of the body and therefore all cancers could be susceptible to this approach including: leukemias, lymphomas, myelomas, glioblastomas, tumors of the lung, ovary, prostate, breast, melanoma, colon, bladder, liver, pancreas, thyroid, head and neck.

In one embodiment, the cell is a leukemia cell. In one embodiment, the cell is a lymphoma cell. In one embodiment, the cell is a myeloma cell. In one embodiment, the cell is a glioblastoma cell. In one embodiment, the cell is lung cancer cell. In one embodiment, the cell is an ovarian cancer cell. In one embodiment, the cell is a prostate cancer cell. In one embodiment, the cell is a breast cancer cell. In one embodiment, the cell is a melanoma cell. In one embodiment, the cell is a colon cancer cell. In one embodiment, the cell is a bladder cancer cell. In one embodiment, the cell is a liver cancer cell. In one embodiment, the cell is a pancreatic cancer cell. In one embodiment, the cell is a thyroid cancer cell. In one embodiment, the cell is a head and neck cancer cell.

In one embodiment, the leukemic cell is an ALL cell. In one embodiment, the leukemic cell is an AML cell. In one embodiment, the leukemic cell is an CLL cell. In one embodiment, the leukemic cell is an CML cell.

Cell lines are optionally transduced or transfected. For example human T cell leukemia Jurkat T cells, human erythro-leukemic K562 cells, CES1, OCIAML1, OCIAML2, OCIAML3, OCIAML4, OCIAML5, OCIAML6, and Raji cells are optionally transduced or transfected with polynucleotides of the described herein. Raji is a burkitts lymphoma line, OCI AML 1 and 2 are acute meylogenous leukemia lines, CES1 is a chronic myelongenous leukemia line.

A cancer cell such as a leukemia cell expresses tumor associated antigens. As demonstrated herein, introduction of IL-12 in combination with IL-21 or IL-18, can augment the immune response when the transduced tumor/cancer cell is introduced into the subject. In one embodiment, the tumor/cancer, optionally leukemia cell, is transduced with a lentiviral construct comprising an IL-12 expression cassette and at least one of an IL-21 or IL-18 expression cassette. Cancer cells are attractive vehicles for expressing IL-12 and one or more of IL-21 or IL-18 as the immune response is self-limiting. Transduced cancer cells elicit an immune response that leads to the eradication of the initiating cell. Cytokine levels are thereby self-limited.

Compositions and vector constructs described herein are usefully introduced into any cell type ex vivo. The compositions and vector constructs described herein may also be introduced into any cell type in vivo.

The population of cells can comprise transduced and non-transduced and/or transfected and non-transfected cells. In one embodiment, at least 0.5%. 1%, 2-5%, 5-10%, 10-15%, 15-20%, 20-25%, 25-30%, 30-40%, 40-50%, 50-60%, 60-70%, 70-80%, 80-90%, 90-95%, 95-99% or more than 99% of cells in the population of cells are transduced or transfected and/or express IL-12 and one or more of IL-21 or IL-18.

In an embodiment, the population of cells comprises at least 1%, for example at least 2%, at least 5%, at least 10%, at least 20% or more, transduced cells secreting at least 500 pg/10⁶ cells/ml/hr 1,000 pg/10⁶ cells/ml/hr, 1,500 pg/10⁶ cells/ml/hr, 2,000 pg/10⁶ cells/ml/hr, 2,500 pg/10⁶ cells/ml/hr, 5,000 pg/10⁶ cells/ml/hr, 7,500 pg/10⁶ cells/ml/hr, 10,000 pg/10⁶ cells/ml/hr, 12,500 pg/10⁶ cells/ml/hr, 15,000 pg/10⁶ cells/ml/hr, 17,500 pg/10⁶ cells/ml/hr or 20,000 pg/10⁶ cells/ml/hr of IL-12. In an embodiment, the ratio of IL-12 to IL-21 or IL 12 to IL-18 may be about 1:1, 2:1, 4:1, 5:1, 10:1, 20:1, 50:1, 100:1, 200:1 or any other suitable ratio for example 1.25:1, 1.5:1 or 1.75:1.

In an embodiment, a suitable ratio of IL-12 to IL-21 or IL-21 to IL-18 provides immunity when less than or about 10%, less than or about 5%, less than or about 2%, less than or about 1%, less than or about 0.5%, less than or about 0.2%, or less than or about 0.1% of the cells are expressing the IL-12/IL-21 or IL-12/IL-18 combination, measured for example in an assay such as those described in the Examples.

The level of IL-12, IL-21, and/or IL-18 expression can be determined by a number of methods including methods known in the art and methods described herein. For example IL-12, IL-21, and/or IL-18 levels can be determined by ELISA, cytokine bead assay, intracellular staining, HPLC and MS/MS, or ELISPOT.

### Compositions

The application describes compositions comprising an IL-12 expression cassette, one or more of an IL-21 or IL-18 expression cassette, and a vector such as a lentiviral vector as described herein. The vector is for providing a coding nucleic acid molecule (e.g. the expression cassette) to a subject such that expression of the molecule in the cells provides the biological activity of the polypeptide encoded by the coding nucleic acid molecule to those cells. A coding nucleic acid as used herein means a nucleic acid or polynucleotide that comprises nucleotides which specify the amino acid sequence, or a portion thereof, of the corresponding protein. A coding sequence may comprise a start codon and/or a termination sequence.

In other embodiments, the composition comprises cells modified with the vector constructs described herein. Such modified cells can be administered using methods known in the art such as intraperitoneal, intravenous, subcutaneous, or stereotactic injections to a variety of sites, direct injections, intramuscularly, etc.

### Pharmaceutical Compositions

The pharmaceutical compositions of this invention used to treat patients having diseases, disorders or abnormal physical states could include an acceptable carrier, auxiliary or excipient.

The pharmaceutical compositions are optionally administered by ex vivo and in vivo methods such as electroporation, DNA microinjection, liposome DNA delivery, and virus vectors that have RNA or DNA genomes including retrovirus vectors, lentivirus vectors, Adenovirus vectors and Adeno-associated virus (AAV) vectors, Semliki Forest Virus. Derivatives or hybrids of these vectors are also useful.

Dosages to be administered depend on patient needs, on the desired effect and on the chosen route of administration. The expression cassettes are optionally introduced into the cells or their precursors using ex vivo or in vivo delivery vehicles such as liposomes or DNA or RNA virus vectors. They are also optionally introduced into these cells using physical techniques such as microinjection or chemical methods such as coprecipitation.

The pharmaceutical compositions are typically prepared by known methods for the preparation of pharmaceutically acceptable compositions which are administered to patients, and such that an effective quantity of the nucleic acid molecule is combined in a mixture with a pharmaceutically acceptable vehicle. Suitable vehicles are described, for example in Remington's Pharmaceutical Sciences (Remington's Pharmaceutical Sciences, Mack Publishing Company, Easton, Pa., USA).

On this basis, the pharmaceutical compositions could include an active compound or substance, such as a nucleic acid molecule, in association with one or more pharmaceutically acceptable vehicles or diluents, and contained in buffered solutions with a suitable pH and isoosmotic with the physiological fluids. The methods of combining vectors comprising the expression cassettes with the vehicles or combining them with diluents is well known to those skilled in the art.

### Methods of Inducing/Enhancing Immune Responses and Methods of Treatments

The methods disclosed herein are useful for inducing and enhancing an immune response in a subject. In one embodiment, the subject has cancer. In another embodiment, the subject is in remission. In a further embodiment, the subject has an increased risk of cancer.

In one embodiment, the application provides a method of inducing or enhancing an immune response in a subject comprising administering a transduced cell or population of cells described herein or a composition comprising said cells.

In another embodiment, the application provides a method of inducing or enhancing a memory immune response in a subject.

In one embodiment, the immune response induced or enhanced is a CD4+ T cell mediated immune response. In one embodiment, the immune response induced or enhanced is a CD8 T cell dependent immune response. In one embodiment, the immune response induced or enhanced is a natural killer (NK) dependent immune response. In one embodiment, the immune response induced or enhanced is a gamma delta T cell dependent immune response

The application also provides a method of delivering IL-12 and one or more of IL-21 or IL-18 to a subject for enhancing cancer treatment comprising:
generating an IL-12 and one or more of IL-21 or IL-18 secreting cell wherein IL-12 and one or more of IL-21 or IL-18 secreted per cell is above a threshold level, within a selected range and/or at a selected ratio; and
introducing an effective number of the generated IL-12 and one or more of IL-21 or IL-18 secreting cells to the subject.

In one embodiment, transduced cells, a population of cells and/or a composition comprising said cells are administered to a subject. In another embodiment, the cells, population of cells and/or composition are administered with an adjuvant. For example, in one embodiment incomplete Freund's adjuvant is used. In addition, the cells, population of cells and/or composition is administered once, or repeated. For example, the cells and or population of cells are administered a second time to boost the immune response and/or increase the amount of IL-12 and one or more of IL-21 or IL-18 delivered.

In one embodiment, cancer cells are obtained from a subject, and genetically modified to express and/or secrete IL-12 and IL-21 or IL-18 above a threshold level, within a selected range and/or at a selected ratio. The transduced cells or population of cells comprising transduced cells is irradiated and administered to the subject. Accordingly, in certain embodiments, clinical use of the modified cells is restricted to the subject from whom the cancer cell was derived. In one embodiment, the cancer cells are leukemic cells.

The cells or population of cells may be enriched for transduced cells prior to being administered to the subject. In an embodiment, CD271 positive cells are isolated or enriched prior to being administered.

The cancer cells, such as the leukemic cells, may be autologous or allogenic.

In an embodiment the cancer cells used to produce the recombinant cells are autologous cells.

In one embodiment, the cancer cells are allogenic cancer cells having cancer antigens shared with cancer cells of the subject and modified to secrete IL-12 and one or more of IL-21 and IL-18.

In one embodiment, the leukemia cells are allogenic leukemia cells having leukemic antigens shared with the leukemia cells of the subject and modified to secrete IL-12 and one or more of IL-21 and IL-18. Accordingly, in certain embodiments, clinical use of the cytokine secreting cells is not restricted to the subject from whom the cancer cells were derived.

Wherein cells additionally express an activator polynucleotide encoding a polypeptide that converts a prodrug to a drug, for example a tmpk polynucleotide, optionally a mutant tmpk polynucleotide, cells are optionally not irradiated. Any unwanted cells can be killed upon administration of the prodrug. For example, in some cases, irradiation may negatively effect the ability of the transduced cells to induce an immune response e.g. irradiation may cause cell death in certain cell populations. Use of an activator polynucleotide or other mechanism to remove unwanted cells transplanted into the subject is alternatively used in such situations.

The methods disclosed herein are useful for treating a variety of cancers. For example, leukemias of a variety of types are expected to be amenable to the IL-12 and at least one of IL-21 or IL-18 cellular immunization treatment described herein. For example, the leukemia can be lymphoblastic leukemia (ALL), chronic lymphoblastic leukemia (CLL), chronic myeloid leukemia (CML), or acute myeloid leukemia (AML).

In an embodiment, the leukemia is ALL. In an embodiment, the leukemia is CLL. In one embodiment, the leukemia is CML. In one embodiment, the leukemia is AML.

Residual disease which can lay dormant during remissions may be targeted by the method disclosed herein. The delayed disease progression of many leukemias provides a critical window of opportunity for immune-based approaches. The present immunotherapy may also rid quiescent cells such as cancer initiating “stem” cells because it does not require biochemically or genetically active targets. Further the present immunotherapy may also lead to eradicating metastatic disease.

The methods described herein are also useful to treat solid cancers. For example the methods may be used to treat ovarian cancer, melanoma, renal cancer prostate cancer, and/or glioblastoma. Ovarian cancer cells can be isolated for example from ascites. The immune system is able to seek out cells residing in nearly all parts of the body and therefore all cancers could be susceptible to this approach including: leukemias, lymphomas, myelomas, glioblastomas, tumors of the lung, ovary, prostate, breast, melanoma, colon, bladder, liver, pancreas, thyroid, head and neck. In one embodiment, the cancer is a lymphoma. In one embodiment, the cancer is a myeloma. In one embodiment, the cancer is a glioblastoma. In one embodiment, the cancer is a melanoma. In one embodiment, the cancer is a tumor of the lung. In one embodiment, the cancer is ovarian cancer. In one embodiment, the cancer is a prostate cancer. In one embodiment, the cancer is a breast cancer. In one embodiment, the cancer is a colon cancer. In one embodiment, the cancer is a bladder cancer. In one embodiment, the cancer is a liver cancer. In one embodiment, the cancer is a pancreas cancer. In one embodiment, the cancer is a thyroid cancer. In one embodiment, the cancer is a head or neck cancer.

The cells may be introduced by a variety of routes as disclosed elsewhere including intraperitoneal injection or intravenous infusion. Alternatively, a vector construct, isolated virus or composition comprising said construct or virus can be injected intratumorally such that transduction takes place in vivo

The number of cells injected or administered is in one embodiment an effective number to induce an immune response. For example, the number of cells may be 5×10⁶, 10⁷, 2×10⁷, 4×10⁷, 8×10⁷, 10⁸, or more cells. An immune response can be detected using a number of methods known in the art including detecting host T cell recognition of tumor cells in vitro. Alternatively, an immune response can be detected by assessing cytokine profile changes. For example increased expression of IFN-gamma is indicative of an immune response.

In certain embodiments, the methods further comprise monitoring cancer progression. Cancer progression can be monitored using known methods. In some embodiments, a second infusion is administered, if for example monitoring demonstrates an incomplete response or the patient relapses.

In one embodiment, compositions and vectors of the invention are used to treat cancer by adoptive therapy. In one embodiment, cytotoxic lymphocyte cells such as blast cells are transfected or transduced to express IL-12 and one or more of IL-21 and IL-18 (optionally using a LV-IL-12/IL-21 or a LV-IL12/IL-18 construct) in vitro. Adoptive therapy or adoptive (immuno) therapy refers to the passive transfer of immunologically competent tumor-reactive cells into the tumor-bearing host to, directly or indirectly, mediate tumor regression. The feasibility of adoptive (immuno) therapy of cancer is based on two fundamental observations. The first of these observations is that tumor cells express unique antigens that can elicit an immune response within the syngeneic (genetically identical or similar especially with respect to antigens or immunological reactions) host. The other is that the immune rejection of established tumors can be mediated by the adoptive transfer of appropriately sensitized lymphoid cells. Clinical applications include transfer of peripheral blood stem cells following non-myeloablative chemotherapy with or without radiation in patients with lymphomas, leukemias, and solid tumors.

In one embodiment, autologous DC and T cells are contacted ex vivo with IL-12 and IL-21 or IL-18 transduced cancer cells and/or expanded ex vivo and administered to a subject in need thereof with or without IL-12 and one or more of IL-21 or IL-18 secreting cells.

The compositions and vectors are also useful for the reduction of cell proliferation, for example for treatment of cancer. The present disclosure also provides methods of using compositions and vectors of the disclosure for expressing IL-12 and one or more of IL-21 or IL-18 for the reduction of cell proliferation, for example for treatment of cancer.

The application also provides a method of reducing the number of tumor cells or cancer burden in a subject with cancer, or having an increased likelihood of developing cancer comprising administering a transduced cell, population of cells, or a composition comprising said cells to the subject.

In another embodiment, the application provides a method of treating a subject with cancer or an increased risk of developing cancer comprising administering a transduced cell, population of cells, or a composition comprising said cells to the subject.

Vector constructs containing the nucleic acid molecules of the disclosure and isolated viruses are typically administered to mammals, preferably humans, using techniques described below. The polypeptides produced from the nucleic acid molecules are also optionally administered to mammals, preferably humans. One aspect relates to a method of medical treatment of a mammal in need thereof, preferably a human, by administering to the mammal a vector construct described herein or a cell containing the vector construct.

One aspect relates to methods for providing a coding nucleic acid molecule to the cells of an individual such that expression of the coding nucleic acid molecule in the cells provides the biological activity or phenotype of the polypeptide encoded by the coding nucleic acid molecule. The method also relates to a method for providing an individual having a disease, disorder or abnormal physical state with a biologically active polypeptide by administering a nucleic acid molecule of the present invention. The method may be performed ex vivo or in vivo. Gene therapy methods and compositions are demonstrated, for example, in U.S. Pat. Nos. 5,869,040, 5,639,642, 5,928,214, 5,911,983, 5,830,880,5,910,488, 5,854,019, 5,672,344, 5,645,829, 5,741,486, 5,656,465, 5,547,932, 5,529,774, 5,436,146, 5,399,346 and 5,670,488, 5,240,846. The amount of polypeptide will vary with the subject's needs. The optimal dosage of vector may be readily determined using empirical techniques, for example by escalating doses (see U.S. Pat. No. 5,910,488 for an example of escalating doses).

The method also relates to a method for producing a stock of recombinant virus by producing virus suitable for gene therapy comprising modified DNA encoding a gene of interest. This method preferably involves transfecting cells permissive for virus replication (the virus containing therapeutic gene) and collecting the virus produced.

Cotransfection (DNA and marker on separate molecules) may be employed (see eg U.S. Pat. Nos. 5,928,914 and 5,817,492). As well, a detection cassette or marker (such as Green Fluorescent Protein marker or a derivative) may be used within the vector itself (preferably a viral vector).

### Combination Treatments

In certain embodiments, the vector constructs, transduced cells, population of cells and or compositions comprising these, are administered in combination with other therapies. For example, the vector constructs, transduced cells, population of cells and or compositions comprising these may be administered before or after chemotherapy suitable for the cancer being treated. In other embodiments wherein the cancer is a solid cancer, the vector constructs, transduced cells, population of cells and or compositions comprising these are administered before or after surgery.

In one embodiment, cancer cells are harvested from a subject's blood before the combination treatment, optionally chemotherapy, is started. The cancer cells are then transduced with a LV-IL-12/IL-21 or LV-IL-12/IL-18. Transduced cells may be frozen for later use and optionally administered when the subject is in remission.

The combination treatment can include for example kinase inhibitors, such as checkpoint blockers and/or treatments described in the Examples. For example, for the treatment of leukemias, the combination treatment may include Alemtuzumab (Campath®), daclizumab and denileukin diftitox (Ontak®), MK0457 and Bortezomib (Velcade®), Dasatinib (Sprycel®), or Nilotinib (Tasigna®), Other combinations can be tailored for the specific cancer type. In an embodiment, the combination treatment can include without limitation a kinase inhibitor and/or a checkpoint inhibitor,

### Dosing

The methods provide in certain embodiments, that a composition, transduced cell, population or cells, or vector construct described herein is administered to the subject. The compositions, cells or vector constructs of the present application may be administered at least once a week in one embodiment. However, in another embodiment, the composition, transduced cell, population or cells, or vector construct may be administered to the subject from about one time per week, one time per 14 days, or 28 days. The administration may be repeated 1, 2, 3, 4, 5, 6 or more times. In another embodiment, administration is about once daily for a given treatment. In one embodiment, the treatment is chronic treatment and the length of treatment is 1-2 weeks, 2-4 weeks or more than 4 weeks. The treatment regimen can include repeated treatment schedules. It will also be appreciated that the effective amount or dosage of the compound used for the treatment or prophylaxis may increase or decrease over the course of a particular treatment or prophylaxis regime. In some instances, chronic administration may be required.

The number of cells administered varies for example, with the transduction efficiency and/or secretion level of the transduced cell or population of cells.

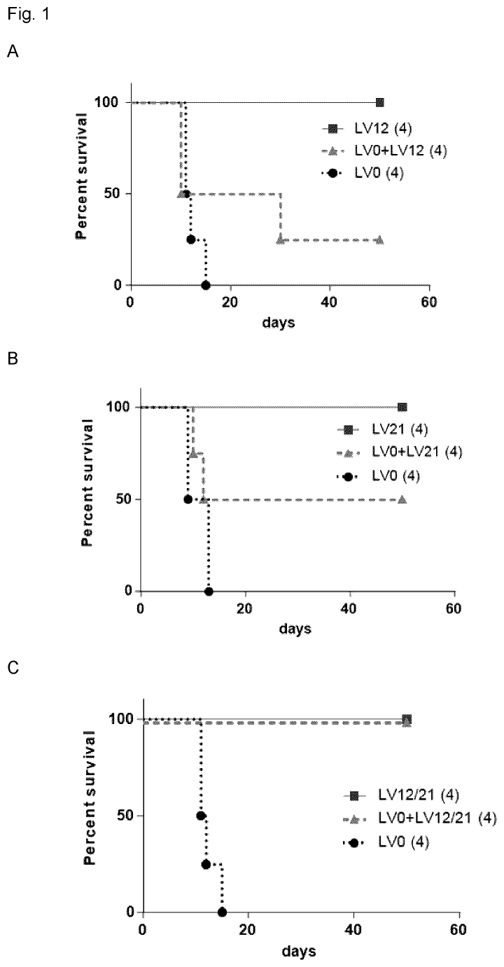

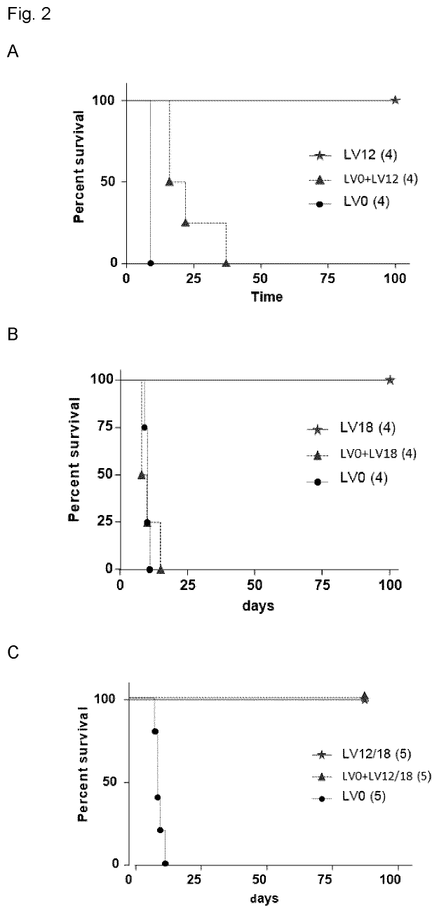

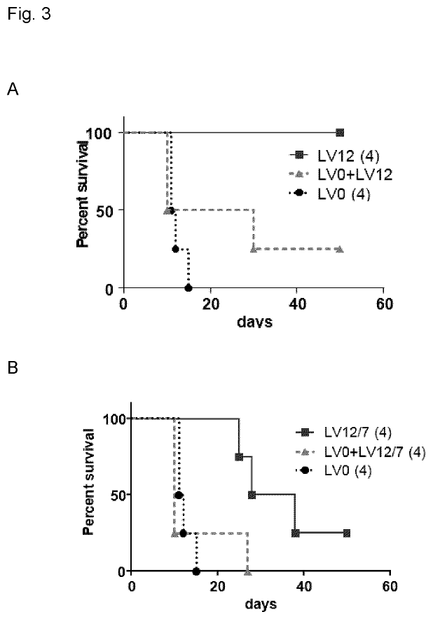

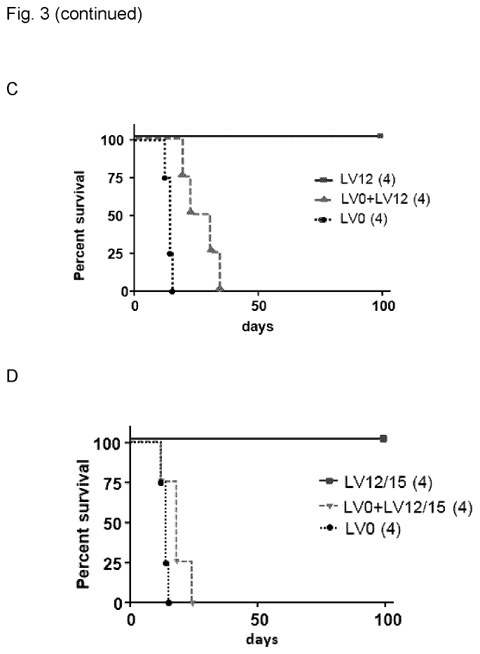

For example, as demonstrated in the Examples, when 100% but not 10% of administered cells are secreting IL-12 at about 1000 pg/10⁶ cells/ml/hr, animals are protected from co-administered untransduced leukemic cells (FIGS. 1A, 2A and 3A). Similarly when 100% but not 10% of administered cells are secreting IL-21 at about 250 pg/10⁶ cells/ml/hr, animals are protected from co-administered untransduced leukemic cells or when 100% but not 10% of administered cells are secreting IL-18 at about 5 pg/mL/10⁶ cells/ml/hr, animals are protected from co-administered untransduced leukemic cells. A lower secretion of IL-12 can be used with increased benefit when cells are secreting both IL-12 and either IL-18 or IL-21. For example, when 10% of administered cells are secreting IL-12 at about 1000 pg/10⁶ cells/ml/hr and also secreting either IL-21 or IL-18, animals are protected from co-administered untransduced leukemic cells (FIGS. 1C and 2C). This is not the case with IL-15 or IL-7.

In one embodiment, 1-5×10⁶, 5-10×10⁶, 10-20×10⁶, 20-30×10⁶, 30-40×10⁶, 40-50×10⁶, 50-60×10⁶, 60-70×10⁶, 70-80×10⁶, 80-90×10⁶, 90-100×10⁶, or more than 100×10⁶ cells are administered. In another embodiment, 10⁶-10⁹ cells are administered.

### Polypeptide Production and Research Tools

A cell line (either an immortalized cell culture or a stem cell culture) transfected or transduced with a polynucleotide of the disclosure (or variants) is useful as a research tool to measure levels of expression of the coding nucleic acid molecule and the activity of the polypeptide encoded by the coding nucleic acid molecule.

An aspect includes a method for producing a recombinant host cell capable of expressing a nucleic acid molecule of the invention comprising introducing into the host cell a vector of the invention.

An aspect also includes a method for expressing a polypeptide in a host cell of the invention including culturing the host cell under conditions suitable for coding nucleic acid molecule expression. The method typically provides the phenotype of the polypeptide to the cell.

Another aspect of the disclosure is an isolated polypeptide produced from a nucleic acid molecule or vector of the invention according to a method of the invention.

Another aspect relates to a system or model for testing the mechanism of IL-12 and IL-21 or IL-18 mediated rejection of cancer. In one embodiment the system is an in vitro system. Understanding the underlying mechanism that leads to an effective anti-leukemia immune response is greatly facilitated by establishing in vitro assays which mimic in vivo observations. This is useful for comparing and adapting murine models to human disease. In one embodiment, the in vitro system comprises murine bone marrow derived DCs (grown for 6-9 days in GM-CSF) induced to mature (increased expression of CD80) in the presence of both spleen cells+70Z/3-IL-12-IL-21 or 70Z/3-IL-12-IL-18 producing cells (but not with either alone). Maturation does not occur if non-transduced 70Z/3 cells are substituted for the 70Z/3-IL-12-IL-21 or 70Z/3-IL-12-IL-18 cells. Selected populations from the spleen are added and/or removed (immature T cells, CD4′ T cells, CD8′ T cells, NKT cells, NK cells, DC precursors) to define the critical cell types that are required for 70Z/3-IL-12-IL-21 or 70Z/3-IL-12-IL-18 mediated DC maturation.

In one embodiment the system comprises human leukemia cells expressing and/or secreting IL-12 and IL-21 or IL-18 and/or a mouse model susceptible to developing cancer to determine the mechanism by which the combination of Interleukin-12 (IL-12) and IL-21 or IL-18 provokes an immune response which, in mice, results in complete rejection of leukemia. In one embodiment, the system permits analysis of the interactions of T cells, dendritic cells (DC), leukemia cells and the cytokines that they produce in established murine in vitro and in vivo systems. In another embodiment, the system permits optimization of the parameters essential for engineering primary samples of human leukemia cells to express quantities of IL-12 and one or more of IL-21 or IL-18 above a threshold level, within a selected range and/or at a selected ratio established in the murine system. In a further embodiment, the system is useful to establish in vitro conditions to determine how primary human leukemia cells expressing IL-12 and IL-21 or IL-18 interact with the autologous DCs and T cells.

### EXAMPLES

### Example 1

### Results

Experiments were undertaken combining each of these cytokines (IL-7,15,18,21) with IL-12. This was accomplished by transducing a clone of 70Z/3 (a murine leukemia) that had previously been transduced with a lentivirus that engineered the expression of IL-12. Such clones are labeled Lentivirus12 (LV12). Clones of LV12 that secreted low amounts of IL-12 were selected. As seen in FIG. 1A, low expressing LV12 clones can induce immunity in mice if 100% of cells are secreting IL-12. However, mixing such clones with the parent line that does not secrete any cytokine (LV0) at a ratio of 1:10 (LV12:LV0) yielded incomplete immunity. It has been previously published that high IL-12 expressing clones (e.g. for example secreting 10,000 pg/10\hat{ }6 cells/ml/hr) provide strong immunity even at 1:200 and in some cases 1:1000.4 FIG. 1B shows that this is also the case for clones that had been transduced with a lentivirus vector that engineers the expression of IL-21. Just like IL-12, lower expressing clones of IL-21 (LV21) only provides strong immunity when present at 100% of the injected populations. To test for cooperation between IL-12 and IL-21 we transduced low expressing LV12 clones with a lentivirus vector that engineers the expression of IL-21 resulting in clones expressing both (LV12+21). Most of the subclones produced very similar amounts of IL-12 as their parent clone (0.75-2 ng/10⁶ cells/ml/hr), but with different levels of IL-21. Clones expressing low amounts of both IL-12 and IL-21 (comparable to the amounts in FIGS. 1A and 1B) were selected and tested for their ability to provide immunity. In this case, as shown in FIG. 1C, strong immunity was found even when mixed with LV0 (1:10).

FIG. 2A-C demonstrates the same result for the combination of IL-12 and IL-18.

Surprisingly, FIG. 2A-C demonstrates that the combination of IL-12 with IL-15 or IL-12 with IL-7 fails to provide evidence for cooperation despite the many well documented examples of cooperation demonstrated in other experimental systems.

TABLE
Approximate expression of cells (all pg/10⁶ cells/ml/hr)

LV12 - ≈1000
LV21- ≈250
LV18 - ≈5
LV 15- ≈80
LV 7 - ≈250

### Materials and Methods

### Animals.

Female (C57Bl/6×DBA/2)F1 mice (referred to as BDF1), 8-12 weeks, old were purchased from the Jackson Laboratories (Bar Harbor, Ma). Mice were kept under sterile conditions in the specific pathogen free (SPF) animal facility at the Ontario Cancer Institute, Princess Margaret Hospital, Toronto, Ontario, Canada. Mice are fed an irradiated diet and autoclaved tap water. Animals are terminated by CO₂ asphyxiation and cervical dislocation. The Animal Care Committee of the Ontario Cancer Institute approved all experimental protocols employed.

### Leukemia Cells.

70Z/3-L leukemia cells (described in[135]), derived from BDF₁ mice, were maintained in IMDM with 5% heat inactivated fetal bovine serum (HYCLONE, South Logan, Utah, USA), 100 μg/mL penicillin-streptomycin or 100 μg/mL kanamycin (GIBCO-Invitrogen), and 5.5×10⁻⁵ M β-mercaptoethanol (referred to as complete IMDM) in a humidified atmosphere at 37° C. and 5% CO₂. Cell concentrations were kept at 5-10×10⁵ cells/mL.

### Lentiviral Vector Construction.

Lentiviral vectors expressing IL-12 cDNA were constructed by a method similar to that described by Yoshimitsu et al.[117] with modification. Plasmid pORF-mIL12 (IL-12elasti(p35::p40) Mouse (p35::p40)) (InvivoGen, San Diego, Calif.) was modified by creating EcoRI and BamHI restriction enzymes sites, upstream and downstream of the IL-12 gene respectively using a QuickChange Site-Directed Mutagenesis Kit (Stratagene, La Jolla, Calif.). This resulting construct was then digested with EcoRI/BamHI (New England Biolabs). Murine IL-12 cDNA was purified after electrophoresis on a 1% agarose gel, and then subcloned into the pHR′ LV backbone downstream of the elongation factor 1 alpha (EF1α) promoter. Positive plasmid clones for pHR-cPPT-EF1α-muIL-12-WPRE (i.e. LV-muIL-12) were identified by diagnostic restriction enzyme digestion analyses and subsequent DNA sequencing (Innobiotech, Toronto, ON, Canada).

Lentiviral vectors expressing murine IL21 or murine IL18 cDNA were generated by Tailored Genes Inc. (Toronto, ON, Canada). The expression vectors were constructed by ligation of the respective cDNA sequences in pRS.EF1a.W. lentivirus expression vector. For construction of muIL-21 lentiviral expression vector, muIL-21 cDNA was first synthesized (BioMatik Corporation, Cambridge, ON, Canada), and then amplified by PCR using muIL-21 specific forward (5′-tagctctagaggatccgccaccatggagaggacccttgtctgt-3) (SEQ ID NO: 8) and reverse (5′-gaggttgattgtcgacctaggagagatgctgaat-3) (SEQ ID NO: 9) primers (ACGT Corporation, ON, Canada). The amplified muIL-21 sequence was purified after electrophoresis on a 1% agarose gel, and subsequently ligated downstream of the elongation factor 1 alpha (EF1a) promoter in the pRS.EF1a.W backbone vector using InFusion Cloning (Takara, Calif., USA). Positive plasmid clones of pRS.EF1a.muIL-21.W. were identified by diagnostic digest and validated by DNA sequencing (ACGT Corporation, Toronto, ON, Canada).

For construction of the vector with muIL-18 with the murine Immunoglobulin kappa (Igkappa) signal sequence (ss) specific forward primer 5′-agctctagaggatccgccaccatggagacagacacactcctgctatgggt actgctgctctgggttccaggttccactggtgacaactttggccgacttcactgtaca-3′ (SEQ ID NO: 10; IDT, Iowa, USA) and reverse primer (5′-gaggttgattgtcgacctaactttgatgtaagttagtgag-3′) (SEQ ID NO: 11; Integrated DNA Technologies, Iowa, USA) were used to amplify the muIL-18 sequence to generate muIgkappa/muIL18.

For construction of vector with muIL-18 with the human IL-2 signal sequence (ss), specific forward primer (5 ‘-tagctctagaggatccgccaccatgtacaggatgcaactcctgtcttgcattgcactaagtcttgcacttgtcacgaattcgaac tttggccgacttcactgtaca-3’) (SEQ ID NO: 12) and reverse primer (5′-gaggttgattgtcgacctaactttgatgtaagttagtgag-3′) (SEQ ID NO: 13; Integrated DNA Technologies, Iowa, USA) were used to amplify the muIL-18 sequence to generate huIL-2ss/muIL18. The muIL-18 cDNA, used as a template for amplification, was purchased from Sino Biological Inc. (Wayne, Pa., USA).

The amplified muIgk/muIL-18 or huIL-2ss/muIL-18 sequences were ligated in the pRS.EF1a.W vector, and positive clones identified, as described above.

For construction of the IL-7 vector, the same procedure was used as for IL-18 and IL-21 except the following primers were used:

Forward primer.
(SEQ ID NO: 14; ACGT Corporation, ON, Canada
5′ tagctctagaggatccgccaccatgttccatgtttcttttaga-3′
Reverse primer
(SEQ ID NO: 15; ACGT Corporation, ON, Canada).
5′ gaggttgattgtcgacttatatactgcccttcaaaat-3′

The IL-15 vector was constructed as follows: The signal sequence and pro-peptide of tissue plasminogen activator (amino acids 1-35 as predicted by Uniprot bioinformatic analyses) replaced the endogenous signal sequence and pro-peptide (amino acids 1-48 as predicted by Uniprot bioinformatic analyses) of mouse IL-15. A DNA cassette comprising a Kozak consensus sequence and this IL-15sol cDNA was synthesized by Genscript (Piscataway, N.J.) and subcloned into the lentiviral backbone pDY.cPPT-EF1α.WPRE downstream of the EF-1a promoter. The vector was verified by restriction enzyme digestion and DNA sequencing.

### Viral Production and Transduction of the Cells.

For IL-12 LV, concentrated LVs were produced by a transient triple-transfection method using pHR-cPPT-EF1α-muIL-12-WPRE and accessory plasmids onto 293T monolayers by calcium phosphate.[136, 137] An approximate vector titre was estimated based on LV/enGFP[117] production and testing on naïve 293T cells that occurred in parallel. The murine pre-B leukemic cell line, 70Z3-L, was then transduced with an approximate multiplicity of infection (MOI) of 20. Single cell clones were obtained by limiting dilution in 96 well plates at population densities of less than 0.3 cells/well.

IL-15 Lentiviral particles were produced at the University Health Network Vector Production Facility. In short, HEK293-T packaging cells were transiently co-transfected with transfer plasmid (LV15) as well as two LV packaging plasmids pCMVΔR8.91 and pMDG. 18 h later a media change was performed. Two supernatant collections were performed, one at 24 h post media change (subsequently held at 4° C. for 24 h), and one at 48 h post media change. Pooled supernatant was filtered through a 0.22 mm Stericup filter (Millipore, Mass.) and ultracentrifuged at 52,800 g, at 4° C. for 2 hours. Vector pellets were resuspended in cell culture media, and frozen until usage.

IL-21 and IL-18 constructs were also made.

Clones were quantitated for cytokine production. IL-12 production/10⁶ cells/mL/2 hrs was measured using a commercially available IL-12 ELISA kit (BD Biosciences, San Jose, Calif.). IL-21 production was measured using a commercially available IL-21 ELISA kit (mouse IL-21 DuoSet ELISA, DY594, R7D Systems, USA). IL-18 production was measured by ELISA using Anti-IL-18 (Mouse) mAb D047-3 Clone 74 (RatIgG2a) and Anti-IL-18 (mouse) mAb-Biotin D048-6 Clone 93-10c (Rat IgG1), both purchased from Medical & Biological Laboratories Co. LTD japan

### Example 2

### Human IL-12, IL-21, and IL 18

### Lentiviral Vector Construction

Lentiviral vectors expressing human IL-12 cDNA were constructed by a method similar to that described for mouse IL-12 construct. The cDNA of human IL-12 was obtained as a fusion form from InvivoGen (pORF-hIL12 (IL-12elasti(p35::p40)). The open reading frame of the gene was amplified by the following PCR primers: hIL-12 ORF Fwd, 5′-TTGGCGCGCCACCATGGGTCACCAGC-3′; (SEQ ID NO: 16) and hIL-12 ORF Rev, 5′-TTGGCGCGCCTTAGGAAGCATTCAGATAGCTCATCACTC-3′ (SEQ ID NO: 1). The PCR product was then subcloned into the Lentiviral backbone (pHR′-cPPT-EF1a-WPRE). The construct was confirmed by diagnostic restriction enzyme digestion analyses and subsequent DNA sequencing.

Lentiviral vectors expressing human IL-21 or human IL-18 cDNA will be constructed by a method similar to that described for human IL-12 construct. The cDNA of human IL-21 or IL-18 will be obtained and the open reading frame of the gene will be amplified by the PCR. The PCR product will be subcloned into a suitable Lentiviral backbone (e.g. pHR′-cPPT-EF1a-WPRE). The construct will be confirmed by diagnostic restriction enzyme digestion analyses and subsequent DNA sequencing.

A bicistronic construct will be created using e.g. the pHR′-cPPT-EF1a-WPRE backbone to combine the fused IL-12 construct (encoding both p40 and p35) with an IL-21 or an IL-18 construct inserted downstream of an IRES site. A second construct will be created in which the positions of the IL-12 and IL-21 or IL-18 constructs relative to the IRES will be reversed. Another set of vectors will be created comprising the IL-12 and one or more of the IL-21 or IL-18 constructs separated by a self-cleaving 2A sequence.

### Transfection

To assess the IL-12/IL-21 and IL-12/IL-18 constructs, 1×10⁶ 293T cells will be transfected with the construct, the human IL-12 template pORF-hIL12, the human IL-21 template, the human IL-18 template or empty lentivector. Cell supernatant will be collected 24 and 48 hours after transfection. The hIL-12, hIL-21, and/or hIL-18 levels will be measured by ELISA (BD pharmingen, San Diego, Calif.).

The activity of the expressed cytokines will be tested in functional assays (IFNγ production by NK92 cells for IL-12 and short term proliferation of OCI-BCL1 cells for IL-21¹⁴²) and compared to human recombinant proteins. Use of 2A peptides results in the addition of a few amino acids to the final product. Immunogenicity of the altered products has been shown to be negligible.¹⁴³

### Transduction of 293T Cells

Lentivirus carrying hIL-12, hIL-21, and/or hIL-18 open reading frames (e.g. LV-hIL-12, LV-hIL-21, LV-hIL-18, LV-hIL-12/hIL-21 or LV-hIL-12/hIL-18) will be produced by a transient triple-transfection method using one of the constructs and accessory plasmids onto 293T monolayers by polyethylenimine. Virus supernatant will be collected 24 and 48 hours after transfection. To test the transduction ability of the lentivirus, 1×10⁶ 293T cells will be transduced with the virus supernatant. hIL-12, hIL-21, and/or h IL-18 expression levels in the cell supernatant will be measured by the same ELISA assay as mentioned above.

### Transduction Protocols

Using the dual cytokine vectors described above, clones will be established using the following transduction protocol. An overnight culture of 1×10⁵ cells will be exposed to LV at an MOI of 10 and supplemented with protamine sulfate (8 ug/ml). After two days cells will be assessed for cytokine expression both in the supernatant (by ELISA) and at the single cell level (by intracellular cytokine staining and FLOW analysis). Experiments will be carried out by varying the following conditions: MOI (1,5,10,20), protamine sulfate concentration, and using alternative transduction supplements such as polybrene or Dextran. In all cases only clinical grade reagents will be used.

### Transduction Protocol for Primary AML Samples.

At least 30 individual primary AML samples will be obtained and tested. This will be done using both freshly obtained (never frozen) samples as well as samples frozen and thawed. Different MOIs, transduction medium and supplements, and exposure time will be tested to determine optimal conditions to reliably transduce a sufficient number of primary AML cells at a sufficient level of cytokine production. Both cytokines should be expressed at high levels in at least some cells. Based on the IL-12 clinical data the transduction of primary AML cells is expected to result in a broad range of cytokine expression levels. However, the IL12/IL21 or IL12/IL18 combination will allow even lower expressing cells to still initiate immune responses.

### Example 3

### Chronic Myeloid Leukemia in Humans

Immunotherapy offers a method to improve the treatment of leukemias, in particular in combination with other treatment modalities. Indeed, maybe only potent immune system-invoking therapy will be effective at fully eradicating leukemia since residual disease often exists in patients that are in remission, which can be re-activated later. This is especially true for chronic myeloid leukemia (CML), a clonal disorder involving the Philadelphia chromosome, which represents 15% of all adult leukemias. On the other hand, this delayed disease progression provides a key window of opportunity for immunotherapy. Since immunotherapy is not dependent on abrogating cell functions by interrupting signaling or on intercalation into DNA by small molecules, for example, it can also be effective on transformed cells that are quiescent or inhabit inaccessible locales. Of importance, immunotherapy may be an effective way to target true cancer stem cells. Lastly, due to the circulating and surveillance nature of the immune system, existing metastatic disease even in primary CML patients could be treated by this approach.

Current first-line therapy involves treatment of CML patients with imatinib mesylate (Gleevec®), a small-molecule tyrosine kinase inhibitor of the Bcr-Abl product. Unfortunately, this is not a curative treatment. Gleevec is the treatment of choice; however side effects, resistance, the need for long-term therapy, and high cost are associated with Gleevec use.

Murine Models of CML.

CML and ALL are similar in that high remission rates in adults are followed by high relapse rates. This clinical course not only provides initial material suitable for infecting with the vector constructs described herein but a rationale for subsequent treatment. Importantly, CML shows this bi/tri-phasic progression and some initial response to imatinib that allows time to develop immune modulating tumor cells following vector transductions.

LVs offer some real advantages over other gene transfer methods that seek to generate stable cell lines secreting IL-12 and IL-21 or IL-12 and IL-18 for such applications: for example—plasmid transfection is very inefficient and adenovirus- or AAV-mediated gene delivery do not lead to appreciable vector integration, which will provide variable levels of cytokine over time. The inventors have shown that transduced murine cells stably express transgenes ˜2 years after initial infection (24).

Synthesis of human vectors. A recombinant LV that engineers stable expression of either human IL-12 and IL-21 or human IL-12 and IL-18 will be generated.

Generation of high-titer vector stocks. High titer recombinant virion stocks were generated and titered in vitro. High titer vector stocks were established by ultracentrifugation of collected and pooled supernatants after triple plasmid transfections of 293T cells as done before (117). The vector was pseudotyped with the VSV-g glycoprotein which allows a wide range of cells to be infected. After sufficient titer of the pHR′human IL-12 delivery vector is obtained, pooled vector stocks will be tested by a ‘Direct’ assay to ensure that RCL has not been generated. In this assay, recipient 293T cells are infected a single time and then grown out for a number of passages. After 4-6 weeks, supernatants from these infected cells are collected and used to infect naïve cells. These cells are grown out and then assayed by functional assays and PCR on isolated genomic DNA to determine if vector has been functionally transmitted to these secondary recipient targets.

Testing in 293T cells. The level of human IL-12, IL-21, and/or IL-18 produced in comparison to vector copy number in infected cells will be determined. Firstly, 293T cells will be infected at a range of modest MOIs from about 0.1 to 100. Supernatants from pools of infected cells, done in triplicate, will be examined for human IL-12, IL-21, and/or IL-18 production by ELISAs. Next, individual cell clones will be established by limiting dilution. These cell lines will examined for human IL-12, IL-21, and/or IL-18 production relative to copies of integrated provirus—as measured by Southern blots. Controls will be comprised of 293T cells infected with a LV/eGFP virus previously constructed (19). This information will provide information relating to the relative MOIs to be used and allows correlation of the secretion of human forms of IL-12, IL-21, and/or IL-18 with relative vector copy number. Use of this stable cell line will provide a reference point for titering all future viral preparations that are made with the intent of infecting patient CML cells, which may have considerable variability in sample-to-sample infection frequencies.

Testing in Human CML. Firstly, established CML cell lines will be infected at various MOIs and clonal populations will be assessed for IL-12, IL-21, and/or IL-18 expression in relation to vector copy number. It has been shown by the inventors that K562 (a CML line) is readily and productively infected with recombinant LVs (21). Numerous clones from each pool will be derived and examined for vector copy and relative human IL-12, IL-21, and/or IL-18 production. Cell viability of clones producing various levels of human IL-12, IL-21, and/or IL-18 over time will be measured by thymidine incorporation assays. Cells will be cultured for many weeks and compared with original clones frozen initially after limiting dilution to determine if human IL-12, IL-21, and/or IL-18 production changes over time. Vector stability will also be measured in these cells by repeat Southern Blot analyses. Secondly, primary human CML cells will be obtained from a minimum of 3-5 CML donors initially to reduce reliance on a single sample. Here cells will be infected at 2 or 3 different MOIs. Cells from each donor will be handled separately to give information on the variability that can be expected. As above, human IL-12, IL-21, and/or IL-18 production will be measured by ELISA in relation to vector copy number.

Additional pre-clinical data will be obtained. From a number of transduced K562 and Jurkat clonal lines, the sequence of the human IL-12, IL-21, and/or IL-18 cDNA from the integrated provirus in genomic DNA will be determined after PCR amplification and subcloning to a stable plasmid. This will provide information on the stability of the vector itself and whether recombinations are occurring that could decrease protein expression levels from a given vector copy number. If consistent alterations are observed in a variety of clones such sequences could be mutated to reduce overlap or alter secondary mRNA structure to favor maintenance of fidelity. Further the vector integration site of cell populations by LM-PCR will be analysed to determine clonality. It will also be important to determine that the human IL-12, IL-21, and/or IL-18 secreted by the transduced CML clones is functional. For this primary human DC cultures will be used to examine stimulation and the enhancement of T cell proliferation compared to controls.

It will be determined whether vector-transduced primary CML cells that have undergone growth arrest (by very high dose irradiation, for example) in preparation for safe clinical infusions into patients are still able to secrete similar levels of human IL-12, IL-21, and/or IL-18 compared to control cells. No differences are expected as others have shown stable expression of GM-CSF and CD40L, for example, in patient leukemia cells after irradiation (122). One group even reported enhanced transgene expression in leukemia cells after γ-irradiation (123). Also, the cell fate control component mentioned above may be added, and killing efficiency of transduced primary CML cells producing human IL-12, IL-21, and/or IL-18 will be assessed after AZT addition at concentrations used previously (118).

Test CML cell growth in vivo. The cell lines are assessed for growth in vivo. Cells will be introduced in immune deficient NOD/SCID mice and mice will be examined for the persistence of transduced CML cell lines and primary patient cells in vivo in this xenograft model. This model shows stable engraftment of human hematopoietic cells, especially when an antibody is given to reduce murine NK cell activity. anti-CD122 antibody (121) from a hybridoma cell line is purified in milligram quantities. Both growth-arrested cells and un-manipulated transduced cells will be given at various doses to recipient NOD/SCID mice. Persistence of transduced CML cells will be determined by conventional assays involving flow cytometry for human cell surface antigens (such as CD45/CD71) along with RT-PCR analyses for the LV as has been done for the Bcr-Abl oncogene fusion (124). These studies will be important to prove that the CML cells comprise the primary populations in the xenografted animals. As well, circulating levels of human-specific IL-12, IL-21, and/or IL-18 will be determined by ELISA; production of secondary cytokines such as IFN-γ is also measured.

Where the vector that engineers expression of a cell fate control component such as tmpk or mutants thereof is employed, the effectiveness of transduced cell killing in vivo can be measured after the addition of AZT to animals—dosing that is below the level of systemic toxicity is described in (118). A fully adaptive transplant system in this xenograft model is developed wherein matching genetically modified cells are returned to animals previously reconstituted with autologous patient hematopoietic components. The optimal dose of IL-12, IL-21 and/or IL-18 relative to immune response is determined. The effect of the addition of other co-stimulatory molecules or alternative cytokines that perturb the immune response invoked either positively or negatively are assessed. Lentivectors that express shRNAs that downregulate expression of important genes that may effect stimulation such as IL-10 are also assessed. The contribution of various populations of hematopoietic cells themselves using depletion and sorting-mediated add-back studies are also assessed.

### Example 4

Leukemia cells from 4 donors from each group (CML, AML, CLL, ALL) will be enriched following Ficoll centrifugation by established protocols. Initially, for AML and ALL patients with high leukocyte (>60 k) and high % blast counts will be carefully selected, in which case we expect enrichments to exceed 95% purity. For CML, patients in blast crisis will be selected to achieve the same result. For CLL mature CLL lymphocytes from patients with very high leukocyte counts (>100 k) will be achieved to achieve this enrichment. In each experiment, the leukemia cell population will be infected at 3 different MOIs using the LV-hIL-12/hIL21 or LV-hIL-12/hIL-18 constructs and a LV/enGFP control. An enzyme-linked immunospot (ELISPOT) assay for use as a readout in these experiments is being developed. The cloned, stable, murine lines produce a range of IL-12, IL-21, and/or IL-18 from 2-40000 pg/10⁶/ml/2 hrs and serve to calibrate the ELISPOT assay by correlating spot size to known secretion levels at the signal cell level, with IL-21 and IL-18 typically at lower levels than IL-12. A similar calibration set will be created with human established cell lines by subcloning after the primary the LV-hIL-12/hIL21 or LV-hIL-12/hIL-18 transduction. The ELISPOT assay will allow quantification of not only the percentage of primary leukemia cells secreting IL-12, IL-21, and/or IL-18 from the transduced vector or vectors, but also will provide a distribution of IL-12, IL-21, and/or IL-18 production levels. The assay will be developed to reliably yield for example 10% of the leukemia cells secreting at least for example 1000 pg/10⁶/ml/hr, or 2000 pg/10⁶/ml/hr or up to 20000 pg/10⁶/ml/hr IL-12 and a suitable amount of IL-21 or IL-18. Primary cells will be frozen and thawed and retested to determine the stability of this distribution. Primary cells will also be irradiated and retested for the production and distribution of IL-12, IL-21, and/or IL-18 levels. Clinical protocols using these populations would serve as autologous cell based vaccines for example to be used to prevent relapse in patients who achieve complete remission (CR).

### Example 5

### Acute Lymphoblastic Leukemia (ALL)

Similarly as described for CML, ALL cells transduced with LV-hIL-12/hIL21 or LV-hIL-12/hIL-18 constructs will be made and tested.

Testing in Human ALL cells. Firstly, established ALL cell lines will be infected at various MOIs and clonal populations will be assessed for IL-12, IL-21, and/or IL-18 expression in relation to vector copy number. It has been shown by the inventors that Jurkat cells (an ALL line) are readily and productively infected with recombinant LVs (21). Numerous clones from each pool will be derived and examined for vector copy and relative human IL-12, IL-21, and/or IL-18 production. Cell viability of clones producing various levels of human IL-12, IL-21, and/or IL-18 over time will be measured by thymidine incorporation assays. Cells will be cultured for many weeks and compared with original clones frozen initially after limiting dilution to determine if human IL-12, IL-21, and/or IL-18 production changes over time. Vector stability will also be measured in these cells by repeat Southern Blot analyses. Secondly, primary human ALL cells are obtained from a minimum of 3-5 ALL donors initially to reduce reliance on a single sample. Here cells are infected at 2 or 3 different MOIs. Cells from each donor are handled separately to give information on the variability that can be expected. As above, human IL-12, IL-21, and/or IL-18 production will be measured by ELISA in relation to vector copy number.

Additional pre-clinical data will be obtained. From a number of transduced K562 and Jurkat clonal lines, the sequence of the human IL-12, IL-21, and/or IL-18 cDNA from the integrated provirus in genomic DNA will be determined after PCR amplification and subcloning to a stable plasmid. This will provide information on the stability of the vector itself and whether recombinations are occurring that could decrease protein expression levels from a given vector copy number. If consistent alterations are observed in a variety of clones such sequences could be mutated to reduce overlap or alter secondary mRNA structure to favor maintenance of fidelity. Further the vector integration site of cell populations by LM-PCR will be analysed to determine clonality. It will also be important to determine that the human IL-12, IL-21, and/or IL-18 secreted by the transduced CML clones is functional. For this primary human DC cultures will be used to examine stimulation and the enhancement of T cell proliferation compared to controls.

It will be determined whether vector-transduced primary ALL cells that have undergone growth arrest (by very high dose irradiation, for example) in preparation for safe clinical infusions into patients are still able to secrete similar levels of human IL-12, IL-21, and/or IL-18 compared to control cells. No differences are expected as others have shown stable expression of GM-CSF and CD40L, for example, in patient leukemia cells after irradiation (122). One group even reported enhanced transgene expression in leukemia cells after γ-irradiation (123). Also, the cell fate control component mentioned above is optionally added, and killing efficiency of bicistronically transduced primary ALL cells producing human IL-12, IL-21, and/or IL-18 will be assessed after AZT addition at concentrations used previously (118).

### Administering IL-12 and One or More of IL-21 or IL-18 Co-Expressing Cells to an ALL Subject

Acute Lymphoblastic Leukemia: It is estimated that 5,200 new patients will be diagnosed with ALL in the US in 2007, and 1,420 will die of the illness. ALL is the most is the most common type of leukemia in children with 61% of diagnoses made in individuals under age 20 (125). The overall 5-year relative survival rate for the period 1996-2003 was 64.0%. There was a slightly positive annual percentage change (0.3%) in ALL incidence for the period of 1985-2005 (125).

Therapy for ALL includes conventional chemotherapy (vincristine, anthracycline, cyclophosphamide, L-asparaginase etc.), radiation therapy and bone marrow transplant. Newer drugs have been developed including clofarabine, nelarabine, and dasatinib, but here responses have been relatively modest and toxicities remain an issue.

Imatinib has also been used in Philadelphia chromosome positive ALL. Imatinib has limited effectiveness in ALL treatment when used as a single agent, but several studies have shown improved outcomes when it is combined with standard chemotherapy (126). Clofarabine (Clolar®) was approved in December of 2004 for pediatric patients with relapsed or refractory ALL overall response rates average 25% (126). Nelarabine (Arranon®) was approved as an orphan drug by the FDA in October, 2005 for treatment of T-cell ALL. Complete responses are reported in 54% of patients with T-cell ALL (126). Approximately 700 ALL patients per year in the US have T-cell ALL (126).

Drugs in development for ALL include Rituximab in Phase III, AMN107 and 852A both in Phase II, Nilotinib (Tasigna®) and AT9283 both in Phase I/II and KW-2449 in Phase I. Cell based therapies such as nonmyeloablative stem cell transplant and allogeneic umbilical cord blood transplantation are also in development. Drugs in trials for specific types of ALL include therapeutics directed to T-cell ALL (T-ALL) such as Alemtuzumab (Campath®), daclizumab and denileukin diftitox (Ontak®) all in Phase II and Similarly, a number of CML drugs in trials for Ph+ALL such as MK0457 and Bortezomib (Velcade®) which are both in Phase II, SKI-606 in Phase I/II and INNO-406 in Phase I.

### Clinical Use

50 ml of heparanized blood is collected from patients following REB approved informed consent. The blood is diluted with 110 ml of alpha medium and aliquoted in to 50 ml conical centrifuge tubes. Ficol hypaque is injected under the blood and the tubes are spun at 1600 rpm at 15 C for 20 minutes. The layer of mononuclear cells is removed and resuspended in 100 ml alpha medium with 5% FCS. The cells are spun at 1000 rpm for 10 minutes and then resuspended in 10 ml alpha medium with 5% FCS. Cells are then counted and frozen for future use or distributed for fresh experiments. This would yield over 1×10⁹ blasts from the peripheral blood of patients.

Blast cells are collected from the subject prior to chemotherapy when they are very high in numbers. The cells or a portion thereof are optionally frozen, for example as described in Example 9. The patient is treated with chemotherapy or other appropriate modality. Cells are then thawed if frozen, infected with one or more LV constructs for expressing IL-12, in combination with IL-21, and/or IL-18 and analyzed for the required level of expression (e.g above a threshold level, within a selected range and/or at a selected ratio). Cells meeting this criteria are optionally irradiated, and reintroduced into the patient.

Where the vector construct comprises a safety gene component, cells are optionally not irradiated.

Further cells are optionally infected prior to freezing.

Administering IL-12 and one or more of IL-21 or IL-18 co-expressing cells to a Subject with CML

Chronic Myeloid Leukemia: It is estimated that 4,570 people in the US will be diagnosed with CML and 490 will die of this illness 2007 (126). There was a negative annual change in incidence (−2.6%) of CML for the period of 1997-2004 (126).

Current preferred first-line therapy involves treatment of CML patients with imatinib mesylate (Gleevec®). Dasatinib (Sprycel®) has recently been introduced as a therapy for CML patients that have failed treatment with imatinib. Nilotinib (Tasigna®) has very recently been approved in the US as a new anti-cancer therapy for CML patients who are resistant or intolerant to treatment with imatinib.

### Clinical Use

50 ml of heparanized blood is collected from patients following REB approved informed consent. The blood is diluted with 110 ml of alpha medium and aliquoted in to 50 ml conical centrifuge tubes. Ficol hypaque is injected under the blood and the tubes are spun at 1600 rpm at 15 C for 20 minutes. The layer of mononuclear cells is removed and resuspended in 100 ml alpha medium with 5% FCS. The cells are spun at 1000 rpm for 10 minutes and then resuspended in 10 ml alpha medium with 5% FCS. Cells are then counted and frozen for future use or distributed for fresh experiments. This would yield over 1×10⁹ blasts from the peripheral blood of patients.

Blast cells are collected from the subject prior to chemotherapy when they are very high in numbers. The cells or a portion thereof are optionally frozen, for example as described in Example 9. The patient is treated with chemotherapy or other appropriate modality. Cells are then thawed if frozen, infected with LV-hIL-12/hIL21 or LV-hIL-12/hIL-18 and analyzed for the required level of expression (e.g above a threshold level, within a selected range and/or at a selected ratio). Cells meeting this criteria are optionally irradiated, and reintroduced into the patient.

Where the vector construct comprises a safety gene component, cells are optionally not irradiated.

Further cells are optionally infected prior to freezing.

### Administering Cells Secreting IL-12 and One or More of IL-21 and IL-18 to a CLL Patient

CLL B-CLL is the most common leukemia of adults with ˜16500 cases annually (Estimates based on American Cancer Society and Canadian Cancer Society Reports). Remissions can be achieved with purine analogues and monoclonal antibody therapy however the diseases invariable progresses. Allogeneic stem cell transplants can be curative but many patients do not qualify for this treatment because of their age. The observation that graft versus leukemia (GVL) responses occur after stem cell transplantation confirms that an anti-leukemia immune response to CLL is possible. The slow progression of B-CLL also makes this disease attractive for immunotherapy approaches.

### Clinical Use

50 ml of heparanized blood is collected from patients following REB approved informed consent. The blood is diluted with 110 ml of alpha medium and aliquoted in to 50 ml conical centrifuge tubes. Ficol hypaque is injected under the blood and the tubes are spun at 1600 rpm at 15 C for 20 minutes. The layer of mononuclear cells is removed and resuspended in 100 ml alpha medium with 5% FCS. The cells are spun at 1000 rpm for 10 minutes and then resuspended in 10 ml alpha medium with 5% FCS. Cells are then counted and frozen for future use or distributed for fresh experiments.

This would yield over 1×10⁹ blasts from the peripheral blood of patients.

Blast cells are collected from the subject prior to chemotherapy when they are very high in numbers. The cells or a portion thereof are optionally frozen, for example as described in Example 9. The patient is treated with chemotherapy or other appropriate modality. Cells are then thawed if frozen, infected with LV-hIL-12/hIL21 or LV-hIL-12/hIL-18 and analyzed for the required level of expression (e.g above a threshold level, within a selected range and/or at a selected ratio). Cells meeting this criteria are optionally irradiated, and reintroduced into the patient.

Where the vector construct comprises a safety gene component, cells are optionally not irradiated.

Further cells are optionally infected prior to freezing.

### Example 7

### Treating Solid Tumors

Solid tumors are removed partially or fully from a subject. The solid tumor is optionally any resectable tumor. The tumor is optionally an ovarian cancer, renal cell cancer, melanoma, prostate cancer, or glioblastoma. Ovarian cancer cells can be obtained for example from ascites fluid.

Single cell suspensions are obtained and cells are transduced or transfected with an IL-12/IL-21 or IL-12/IL-18 vector construct such as LV hIL-12/hIL-21 or LV hIL-12/hIL-18. Transfected or transduced cells are optionally irradiated to induce growth arrest and prevent cell division.

A population of cells including transduced cancer cells is administered to the subject from which the cancer was derived. The population of cells is administered using suitable route of administration, such as intravenously, intradermally or subcutaneously, or optionally into the tumor itself, or into the cavity left after tumor resection, about once a week, once every two weeks, or about once a month for a 3 month period. Approximately 1×10⁶ to 1×10⁸ cells are administered.

The subject is monitored for an anti-cancer immune response and cancer progression.

### Example 8

### Research Models and Systems

Determine the critical aspects of initiating anti-leukemia responses in the murine system. The in vivo induction of anti-leukemia immunity using in vitro models will be studied. DCs mature in culture when exposed to 70Z/3-IL-12 cells only in the presence of spleen cells. Untransduced 70Z/3 cells do not mirror this effect. Selected populations of spleen cells will be systematically removed to determine which spleen cells are responsible for the observed effects. Antibodies specific for subpopulations of T cells, NK cells, and macrophages, will be used in combination with either MACS or FACS for depletion and/or enrichment. These experiments will be conducted in transwell plates which allow the physical separation of the various cell types to identify critical cell-cell interactions. DC maturation (increased expression of CD80) as our prime read out has been used. However, it is possible that DC maturation in the presence of 70Z/3 cells will be followed by activation of specific T cell populations. The in vitro system will be used to determine if T cell responses are initiated and, if so, the nature of those responses. Cytokine production typical of Th1 induction (such as IFNγ) as well as the appearance CD4⁺ and or CD8⁺ mature T cells specific for 70Z/3 cells will be monitored. 70Z/3 specific T cell clones will be expanded and their cell surface phenotype will be characterized. Their cytotoxic potential in Cr⁵¹ release assays using 70Z/3 cells as targets will be tested.

The established in vivo model will also be used to explore the induction of protective immunity. In particular, adoptive transfer experiments will be undertaken to determine if CD4⁺ cells can confer immunity and if so if these cells are CD4⁺ CTL or NKT cells. These cells will be isolated and cloned in vitro after they arise in the mice to establish their growth properties and mechanism of cytotoxicity. By comparing the induction of immunity to AML to our current ALL model, we will study why some cancers are more immunogenic that others.

With this background knowledge IL-12 in combination with IL-21 and/or IL-18 transduction experiments using established human leukemia cell lines representing different classes of leukemia will be initiated. These include K562, CES1, OCIAML1, OCIAML2, Jurkat, Raji. The cell lines will be transduced in bulk culture after which clones will be selected by limiting dilution. The clones will be examined for cell proliferation by thymidine incorporation assays and for IL-12, IL-18 and IL-21 production by ELISA. The stability of the IL-12, IL-18 and IL-21 production will be determined after extended cell culture times as well as after several freeze/thaw cycles. Repeat Southern blot analysis will be used to determine vector copy number and stability as well.

Human in vitro assay. Established cell lines and primary samples will also be used to develop in vitro assays similar to those underway in the murine system. In vitro culture conditions that support human DCs and T cell subsets have been developed. Using these as a starting point the effects of IL-12/IL-18 or IL-12/IL-21 producing cell lines and primary samples in short term assays will be monitored. The ability of IL-12/IL-18 or IL-12/IL-21 producing cell lines and primary leukemia samples to influence the maturation of human DCs in the presence and absence of selected T cell subsets. Cell surface markers such as CD80 for DC maturation and IFNγγ secretion for induction of Th1 responses will be monitored. If evidence of an IR is detected, CD4 and CD8 subsets will be isolated and tested for anti-leukemia cytotoxicity and specificity.

### Example 9

### Clinical Use in AML

### Isolation and Optional Cryopreservation of Patient PBMCs

A suitable volume (e.g. 5-200 ml) of anticoagulant-treated blood will be collected, mixed with an equal volume of Plasma-Lyte A (Baxter, Deerfield, Ill.), and loaded on top of a suitable volume of Ficoll-Paque Premium (GE, Schenectady, N.Y.) in a 50 ml conical centrifuge tube. After centrifugation at 400 g for 30 minutes at 18° C., the layer of mononuclear cells at the interface will be transferred to a 15 ml conical centrifuge tube. Three volumes of Plasma-Lyte A will be added onto the cells and mixed by pipetting. The resulting cell suspension will then be centrifuged at 200 g for 5 minutes at room temperature. The range of cell numbers collected is expected to be 3×10⁷-1×10⁹ cells per 5-200 mLs peripheral blood, depending on the blast count of the patient.

For cryopreservation, the pellet containing mononuclear cells will be resuspended in Human Serum Plus (GEMINI, West Sacramento, Calif.) to a density of 2×10⁸ cells/ml. Equal volumes of freshly prepared 80% Human Serum Plus plus 20% DMSO (Cryoserv, Bioniche PHARMA, Lake Forest, Ill.) will be subsequently added onto the cell suspension dropwise to achieve a final cell density of 1×10⁸ cells/ml. Cells will then be aliquoted into cryovials and transferred into a freezing container. Cells will be stored at −80° C. overnight and transferred to vapor-phase nitrogen for long-term storage.

### Purification of LV and Functional Titer Analyses

LV/IL-12/IL-21 and LV/IL-12/IL-18 will be produced in compliance with Good Manufacturing Practices (GMP) for human clinical trial use. The LV particles will be produced using HEK293T packaging cells, expanded to 4 l culture volume and transiently cotransfected by the LV packaging plasmids (pCMVΔR8.91 and pMDG) and transfer plasmid. LV particles will be harvested twice, yielding a total 8 l of unconcentrated LV-containing supernatant. The LV-containing supernatant will be purified by Mustang Q ion-exchange chromatography, concentrated by tangential flow filtration, and buffer-exchanged into ˜100 ml clinical-grade CTS AIM V Medium.

Aliquots of the final vector product will be retained for quality control analyses, including confirmation of the vector identity by Southern blot analysis, titer by p24 ELISA, and testing for aerobic and anaerobic sterility, mycoplasma levels, endotoxin levels, residual DNA levels, and residual benzonase levels. Functional titer testing of all LV vector preps will be performed by transduction of HEK293T cells using serial dilutions of the vector followed by intracellular staining for the cytokine of interest followed by flow cytometry. This provides an estimate of both the transduction efficiency, and the expression levels of the desired cytokine.

### LV Transductions

If frozen AML cells are used, cells will first be thawed and cells cultured in complete AIM V medium for 48 hours at a density of 8×10⁶ cells/ml. Every 10 ml of cell suspension will be transferred into separate 15 ml conical tubes and 2 ml Ficoll-Paque Premium will be carefully added at the bottom of the cell suspension. After centrifugation at 400 g for 10 minutes at 18° C., the layer of live cells will be drawn out by a pipette and transferred into a 15 ml centrifuge tube. Then three volumes of AIM V medium will be added in and mixed with the cells. The resulting cell suspension will be centrifuged at 200 g for 5 minutes at room temperature and the cell pellet resuspended in 10 ml complete AIM V. Cell count by Trypan blue exclusion will be performed to determine live cell number, and then cells will be pelleted by centrifugation at 200 g for 5 minutes at room temperature.

The cell pellet will be resuspended with desired volume of LV to reach desired MOIs, and supplemented with complete AIM V and protamine sulfate (final concentration at 8 pg/ml) to reach a density of 1×10⁶ cells/ml. During the transductions, cells will be incubated overnight in a humidified incubator at 37° C. with 5% CO₂, then washed and resuspended in fresh culture medium the next morning. For cells receiving two rounds of transductions, the second infection will be started 2 days after cells were washed following the first transduction.

Measurements of Secreted Human IL-12 p70 and Analysis of IL-12 Bioactivity from LV-Transduced Cells

LV-transduced primary leukemia PBMCs will be washed twice and resuspended with prewarmed culture media to a density of 1×10⁶ cells/ml, then incubated at 37° C. with 5% CO₂ for 2 hours. The cell culture supernatant will then be collected and the amount of human IL-12 p70 will be measured with OptEIA Human IL-12 (p70) ELISA Set (BD Biosciences) according to the manufacturer's instructions. IL-21 will measured using human IL-21 DuoSet ELISA, DY8879, (R&D Systems, USA) and IL-18 will be measured using human IL-18 DuoSet ELISA DY318 (R&D Systems, USA).

### Preparation of Patient Cells for Infusion

Cryopreserved patient PBMCs will be thawed and cultured for 2 days in GMP-grade complete AIM V medium. Viable cells will be recovered after isolation from Ficoll-Paque gradient. Live cells (1×10⁷) will be subjected to an overnight LV transduction as described above. The next morning (Day 4), cells will be washed and cultured in fresh medium for two more days. On day 6, cells will be recovered and cell product will be obtained by washing and resuspending cells with infusion buffer (Plasma-Lyte A plus 0.5% human serum). Transduction efficiency and/or IL-12, IL-21, and/or IL-18 expression levels will be determined as described above. Cell product will be subsequently infused back to the patient.

### Example 10

Experiments were undertaken to estimate the relative number of IL-12 and IL-18 expressing cells needed to induce immunity. LV12 clones producing low (under 2,000 pg/ml/10⁶ cells/hr) and high (>10,000 pg/ml/10⁶ cells/hr) amounts of IL-12 were transduced with IL-18 lentivirus to produce LV12+18 clones. The IL-18 used in these experiments was huIL-2ss/muIL18 as described in Example 1.

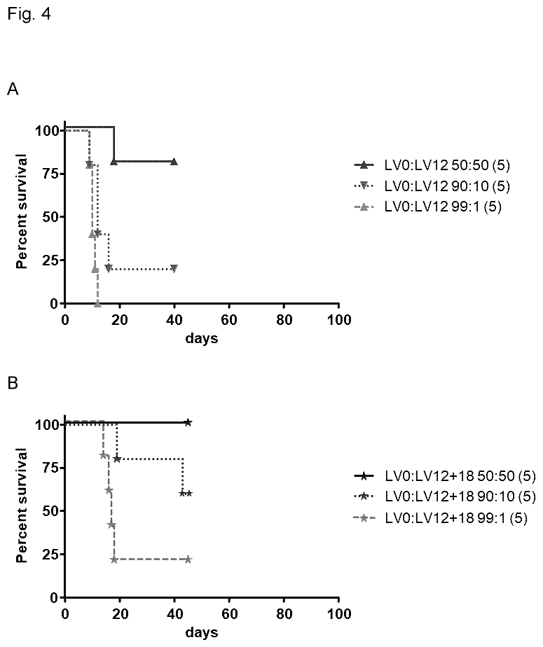

As seen in FIG. 4A, clone LV12 producing low amounts of IL-12 (under 2,000 pg/ml/10⁶ cells/hr) yields incomplete or no immunity when mixed with untransduced (LV0) cells at ratios of 50:50 or lower. However, as seen in FIG. 4B, clone LV12+18 producing low amounts of IL-12 (under 2,000 pg/ml/10⁶ cells/hr)+low amounts of IL-18 (under 2,000 pg/ml/10⁶ cells/hr) provides complete immunity when mixed with untransduced (LV0) cells at a ratio of 50:50, and partial immunity at ratios of 10:90 and 1:99. Cells producing low amounts of both IL-12 and IL-18 show improved potency compared to cells producing low amounts of IL-12 alone.

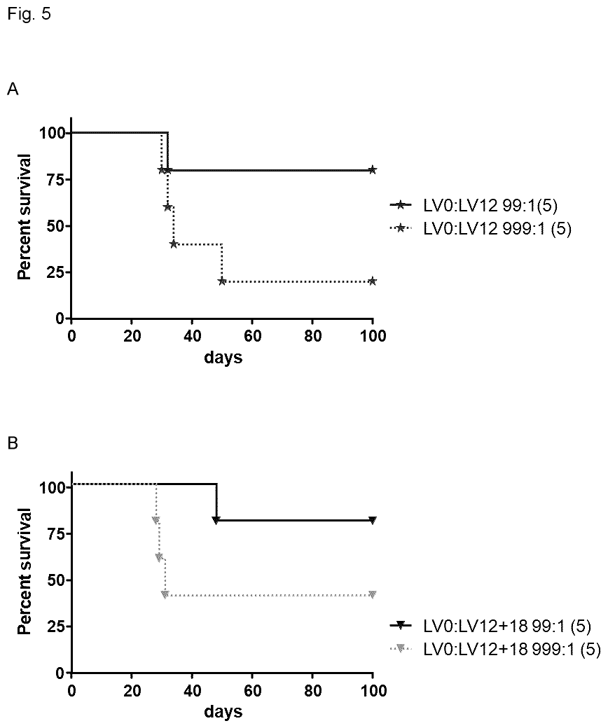

As seen in FIG. 5A, clone LV12 producing high amounts of IL-12 (>10,000 pg/ml/10⁶ cells/hr) yields incomplete immunity when mixed with untransduced (LV0) cells at ratios of 1:99 or 1:999. However, as seen in FIG. 5B, clone LV12+18 producing high amounts of IL-12 (>10,000 pg/ml/10⁶ cells/hr)+high amounts of IL-18 (>5,000 pg/ml/10⁶ cells/hr) provides little difference in immunity when mixed with untransduced (LV0) cells at ratios of 1:99 and 1:999 when compared with cells producing high amounts of IL-12 alone.

Exemplary Sequences

```
hIL-12 ORF Rev (SEQ ID NO: 1)
TTGGCGCGCCTTAGGAAGCATTCAGATAGCTCATCACTC
SEQ ID NO: 2 cPPT seq
ttttaaaaga aaagggggga ttggggggta cagtgcaggg gaaagaatag tagacataat 60
agcaacagac atacaaacta aagaattaca aaaacaaatt acaaaaattc aaaatttt 118
SEQ ID NO: 3
Woodchuck Hepatitus Virus wpre
aatcaacctc tggattacaa aatttgtgaa agattgactg gtattcttaa ctatgttgct 60
ccttttacgc tatgtggata cgctgcttta atgcctttgt atcatgctat tgcttcccgt 120
atggctttca ttttctcctc cttgtataaa tcctggttgc tgtctcttta tgaggagttg 180
tggcccgttg tcaggcaacg tggcgtggtg tgcactgtgt ttgctgacgc aacccccact 240
ggttggggca ttgccaccac ctgtcagctc ctttccggga ctttcgcttt ccccctccct 300
attgccacgg cggaactcat cgccgcctgc cttgcccgct gctggacagg ggctcggctg 360
ttgggcactg acaattccgt ggtgttgtcg gggaagctga cgtcctttcc atggctgctc 420
gcctgtgttg ccacctggat tctgcgcggg acgtccttct gctacgtccc ttcggccctc 480
aatccagcgg accttccttc ccgcggcctg ctgccggctc tgcggcctct tccgcgtctt 540
cgccttcgcc ctcagacgag tcggatctcc ctttgggccg cctccccgcc tg 592
SEQ ID NO: 4
hIL-12 elasti (p40:p35) ORF
atgggtcacc agcagttggt catctcttgg ttttccctgg tttttctggc atctcccctc
gtggccatat gggaactgaa gaaagatgtt tatgtcgtag aattggattg gtatccggat
gcccctggag aaatggtggt cctcacctgt gacacccctg aagaagatgg tatcacctgg
accttggacc agagcagtga ggtcttaggc tctggcaaaa ccctgaccat ccaagtcaaa
gagtttggag atgctggcca gtacacctgt cacaaaggag gcgaggttct aagccattcg
ctcctgctgc ttcacaaaaa ggaagatgga atttggtcca ctgatatttt aaaggaccag
aaagaaccca aaaataagac ctttctaaga tgcgaggcca agaattattc tggacgtttc
acctgctggt ggctgacgac aatcagtact gatttgacat tcagtgtcaa aagcagcaga
ggctcttctg acccccaagg ggtgacgtgc ggagctgcta cactctctgc agagagagtc
agaggggaca acaaggagta tgagtactca gtggagtgcc aggaggacag tgcctgccca
gctgctgagg agagtctgcc cattgaggtc atggtggatg ccgttcacaa gctcaagtat
gaaaactaca ccagcagctt cttcatcagg gacatcatca aacctgaccc acccaagaac
ttgcagctga agccattaaa gaattctcgg caggtggagg tcagctggga gtaccctgac
acctggagta ctccacattc ctacttctcc ctgacattct gcgttcaggt ccagggcaag
agcaagagag aaaagaaaga tagagtcttc acggacaaga cctcagccac ggtcatctgc
cgcaaaaatg ccagcattag cgtgcgggcc caggaccgct actatagctc atcttggagc
gaatgggcat ctgtgccctg cagtgttcct ggagtagggg tacctggggt gggcgccaga
aacctccccg tggccactcc agacccagga atgttcccat gccttcaccca ctcccaaac
ctgctgaggg ccgtcagcaa catgctccag aaggccagac aaactctaga attttaccct
tgcacttctg aagagattga tcatgaagat atcacaaaag ataaaaccag cacagtggag
gcctgtttac cattggaatt aaccaagaat gagagttgcc taaattccag agagacctct
ttcataacta atgggagttg cctggcctcc agaaagacct cttttatgat ggccctgtgc
cttagtagta tttatgaaga ctcgaagatg taccaggtgg agttcaagac catgaatgca
aagcttctga tggatcctaa gaggcagatc tttctagatc aaaacatgct ggcagttatt
gatgagctga tgcaggccct gaatttcaac agtgagactg tgccacaaaa atcctccctt
gaagaaccgg atttttataa aactaaaatc aagctctgca tacttcttca tgctttcaga
attcgggcag tgactattga tagagtgatg agctatctga atgcttccta a 1611
SEQ ID NO: 5
PORF-mIL-12 (p35p40) sequence
atgggtcaat cacgctacct cctctttttg gccacccttg ccctcctaaa ccacctcagt 60
ttggccaggg tcattccagt ctctggacct gccaggtgtc ttagccagtc ccgaaacctg 120
ctgaagacca cagatgacat ggtgaagacg gccagagaaa agctgaaaca ttattcctgc 180
actgctgaag acatcgatca tgaagacatc acacgggacc aaaccagcac attgaagacc 240
tgtttaccac tggaactaca caagaacgag agttgcctgg ctactagaga gacttcttcc 300
acaacaagag ggagctgcct gcccccacag aagacgtctt tgatgatgac cctgtgcctt 360
ggtagcatct atgaggactt gaagatgtac cagacagagt tccaggccat caacgcagca 420
cttcagaatc acaaccatca gcagatcatt ctagacaagg gcatgctggt ggccatcgat 480
gagctgatgc agtctctgaa tcataatggc gagactctgc gccagaaacc tcctgtggga 540
gaagcagacc cttacagagt gaaaatgaag ctctgcatcc tgcttcacgc cttcagcacc 600
cgcgtcgtga ccatcaacag ggtgatgggc tatctgagct ccgccgttcc tggagtaggg 660
gtacctggag tgggcggatc tatgtgggag ctggagaaag acgtttatgt tgtagaggtg 720
gactggactc ccgatgcccc tggagaaaca gtgaacctca cctgtgacac gcctgaagaa 780
gatgacatca cctggacctc agaccagaga catggagtca taggctctgg aaagaccctg 840
accatcactg tcaaagagtt tctagatgct ggccagtaca cctgccacaa aggaggcgag 900
actctgagcc actcacatct gctgctccac aagaaggaaa atggaatttg gtccactgaa 960
attttaaaaa atttcaaaaa caagactttc ctgaagtgtg aagcaccaaa ttactccgga 1020
cggttcacgt gctcatggct ggtgcaaaga aacatggact tgaagttcaa catcaagagc 1080
agtagcagtc cccccgactc tcgggcagtg acatgtggaa tggcgtctct gtctgcagag 1140
aaggtcacac tggaccaaag ggactatgag aagtattcag tgtcctgcca ggaggatgtc 1200
acctgcccaa ctgccgagga gaccctgccc attgaactgg cgttggaagc acggcagcag 1260
aataaatatg agaactacag caccagcttc ttcatcaggg acatcatcaa accagacccg 1320
cccaagaact tgcagatgaa gcctttgaag aactcacagg tggaggtcag ctgggagtac 1380
cctgactcct ggagcactcc ccattcctac ttctccctca agttctttgt tcgaatccag 1440
cgcaagaaag aaaagatgaa ggagacagag gaggggtgta accagaaagg tgcgttcctc 1500
gtagagaaga catctaccga agtccaatgc aaaggcggga atgtctgcgt gcaagctcag 1560
gatcgctatt acaattcctc atgcagcaag tgggcatgtg ttccctgcag ggtccgatcc 1620
tag
hIL-18-Uniprot accession number Q14116 (SEQ ID NO: 6)
                10 20 30 40 50
MAAEPVEDNC INFVAMKFID NTLYFIAEDD ENLESDYFGK LESKLSVIRN
                60 70 80 90 100
LNDQVLFIDQ GNRPLFEDMT DSDCRDNAPR TIFIISMYKD SQPRGMAVTI
              110 120 130 140 150
SVKCEKISTL SCENKIISFK EMNPPDNIKD TKSDIIFFQR SVPGHDNKMQ
              160 170 180 190
FESSSYEGYF LACEKERDLF KLILKKEDEL GDRSIMFTVQ NED
hIL-21-Uniprot accession number Q9HBE4 (SEQ ID NO: 7)
                10 20 30 40 50
MRSSPGNMER IVICLMVIFL GTLVHKSSSQ GQDRHMIRMR QLIDIVDQLK
                60 70 80 90 100
NYVNDLVPEF LPAPEDVETN CEWSAFSCFQ KAQLKSANTG NNERIINVSI
              110 120 130 140 150
KKLKRKPPST NAGRRQKHRL TCPSCDSYEK KPPKEFLERF KSLLQKMIHQ
              160
HLSSRTHGSE DS
mulL-21 forward (SEQ ID NO: 8)
tagctctagaggatccgccaccatggagaggacccttgtctgt
mulL-21 reverse (SEQ ID NO: 9)
gaggttgattgtcgacctaggagagatgctgaat
murine Igkappa signal sequence (ss) forward primer (SEQ ID NO: 10)
agctctagaggatccgccaccatggagacagacacactcctgctatgggtactgctgctctgggttccag
gttccactggtgacaactttggccgacttcactgtaca
murine Igkappa signal sequence (ss) reverse primer (SEQ ID NO: 11)
gaggttgattgtcgacctaactttgatgtaagttagtgag
human IL-2 signal sequence (ss), forward primer (SEQ ID NO: 12)
tagctctagaggatccgccaccatgtacaggatgcaactcctgtcttgcattgcactaagtcttgcactt
gtcacgaattcgaactttggccgacttcactgtaca
human IL-2 signal sequence (ss), reverse primer (SEQ ID NO: 13)
gaggttgattgtcgacctaactttgatgtaagttagtgag
IL-7 forward primer (SEQ ID NO: 14)
tagctctagaggatccgccaccatgttccatgtttcttttaga
IL-7 reverse primer (SEQ ID NO: 15)
gaggttgattgtcgacttatatactgcccttcaaaat
hIL-12 ORF Fwd (SEQ ID NO: 16)
TTGGCGCGCCACCATGGGTCACCAGC
hIL-2 signal sequence (nucleic acid sequence)
ATGTACAGGATGCAACTCCTGTCTTGCATTGCACTAAGTCTTGCACTTGTCACGAATTCG
(SEQ ID NO: 17)
hIL-2 signal sequence (amino acid sequence)
MYRMQLLSCIALSLALVTNS (SEQ ID NO: 18)
```

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCES

1. Trinchieri G. Interleukin-12 and the regulation of innate resistance and adaptive immunity. Nat Rev Immunol. 2003; 3:133-146.
2. Del Vecchio M, Bajetta E, Canova S, Lotze M T, Wesa A, Parmiani G, Anichini A. Interleukin-12: biological properties and clinical application. Clin Cancer Res. 2007; 13:4677-4685.
3. Ohteki T, Fukao T, Suzue K, Maki C, Ito M, Nakamura M, Koyasu S. Interleukin 12-dependent interferon gamma production by CD8alpha+ lymphoid dendritic cells. J Exp Med. 1999; 189:1981-1986.
4. Labbe A, Nelles M, Walia J, Jia L, Furlonger C, Nonaka T, Medin J A, Paige C J. IL-12 immunotherapy of murine leukaemia: comparison of systemic versus gene modified cell therapy. J Cell Mol Med. 2009; 13:1962-1976.
5. Wei L Z, Xu Y, Nelles E M, Furlonger C, Wang J C, Di Grappa M A, Khokha R, Medin J A, Paige C J. Localized interleukin-12 delivery for immunotherapy of solid tumours. J Cell Mol Med. 2013; 17:1465-1474.
6. Nelles M E, Moreau J M, Furlonger C L, Berger A, Medin J A, Paige C J. Murine Splenic CD4+ T Cells, Induced by Innate Immune Cell Interactions and Secreted Factors, Develop Antileukemia Cytotoxicity. Cancer Immunol Res. 2014.
7. Nelles M, Salerno, V., Xu, Y., Paige C J. Cytokine Immunotherapy. In: Fowler JAMaDH, ed. Experimental and Applied Immunotherapy: Humana Press; 2011:281-306.
8. Huang J, Liu Y, Au B C, Barber D L, Armda A, Schambach A, Rothe M, Minden M D, Paige C J, Medin J A. Preclinical validation: LV/IL-12 transduction of patient leukemia cells for immunotherapy of AML. Mol Ther Methods Clin Dev. 2016; 3:16074.
9. Di Carlo E, Comes A, Basso S, De Ambrosis A, Meazza R, Musiani P, Moelling K, Albini A, Ferrini S. The combined action of IL-15 and IL-12 gene transfer can induce tumor cell rejection without T and NK cell involvement. J Immunol. 2000; 165:3111-3118.
10. Orengo A M, Di Carlo E, Comes A, Fabbi M, Piazza T, Cilli M, Musiani P, Ferrini S. Tumor cells engineered with IL-12 and IL-15 genes induce protective antibody responses in nude mice. J Immunol. 2003; 171:569-75.
11. Tian Y, Yuan C, Ma D, Zhang Y, Liu Y, Zhang W, Hou F, Cui B. IL-21 and IL-12 inhibit differentiation of Treg and TH17 cells and enhance cytotoxicity of peripheral blood mononuclear cells in patients with cervical cancer. Int J Gynecol Cancer. 2011; 21:1672-8.
12. Yu S, Jia L, Zhang Y, Zhong J, Yang B, Wu C. IL-12 induced the generation of IL-21- and IFN-gamma-co-expressing poly-functional CD4+ T cells from human naive CD4+ T cells. Cell Cycle. 2015; 14:3362-72.
13. Shi L, Chen S, Zha X, Xu Y, Xu L, Yang L, Lu Y, Zhu K, Li Y. Enhancement of the TCRzeta expression, polyclonal expansion, and activation of t cells from patients with acute myeloid leukemia after IL-2, IL-7, and IL-12 induction. DNA Cell Biol. 2015; 34:481-8.
14. Freeman B E, Hammarlund E, Raue H P, Slifka M K. Regulation of innate CD8+ T-cell activation mediated by cytokines. Proc Natl Acad Sci USA. 2012; 109:9971-6.
15. Kobayashi M, Fitz L, Ryan M, Hewick R M, Clark S C, Chan S, Loudon R, Sherman F, Pemssia B, Trinchieri G. Identification and purification of natural killer cell stimulatory factor (NKSF), a cytokine with multiple biologic effects on human lymphocytes. J Exp Med. 1989; 170:827-845.
16. Stern A S, Podlaski F J, Hulmes J D, Pan Y C, Quinn P M, Wolitzky A G, Familletti P C, Stremlo D L, Truitt T, Chizzonite R, et al. Purification to homogeneity and partial characterization of cytotoxic lymphocyte maturation factor from human B-lymphoblastoid cells. Proc Natl Acad Sci USA. 1990; 87:6808-6812.
17. Sun L, He C, Nair L, Yeung J, Egwuagu C E. Interleukin 12 (IL-12) family cytokines: Role in immune pathogenesis and treatment of CNS autoimmune disease. Cytokine. 2015; 75:249-55.
18. Leonard W J, Wan C K. IL-21 Signaling in Immunity F1000Res. 2016; 5.
19. Davis M R, Zhu Z, Hansen D M, Bai Q, Fang Y. The role of IL-21 in immunity and cancer. Cancer Lett. 2015; 358:107-14.
20. Xu M, Liu M, Du X, Li S, Li H, Li X, Li Y, Wang Y, Qin Z, Fu Y X, Wang S. Intratumoral Delivery of IL-21 Overcomes Anti-Her2/Neu Resistance through Shifting Tumor-Associated Macrophages from M2 to M1 Phenotype. J Immunol. 2015; 194:4997-5006.
21. Kumano M, Ham I, Furukawa J, Oniki S, Nagai H, Miyake H, Fujisawa M. Interleukin-21 activates cytotoxic T lymphocytes and natural killer cells to generate antitumor response in mouse renal cell carcinoma. J Urol. 2007; 178:1504-9.
22. Ugai S, Shimozato O, Kawamura K, Wang Y Q, Yamaguchi T, Saisho H, Sakiyama S, Tagawa M. Expression of the interleukin-21 gene in murine colon carcinoma cells generates systemic immunity in the inoculated hosts. Cancer Gene Ther. 2003; 10:187-92.
23. Ma H L, Whitters M J, Konz R F, Senices M, Young D A, Grusby M J, Collins M, Dunussi-Joannopoulos K. IL-21 activates both innate and adaptive immunity to generate potent antitumor responses that require perforin but are independent of IFN-gamma J Immunol. 2003; 171:608-15.
24. Bamford R N, Grant A J, Burton J D, Peters C, Kurys G, Goldman C K, Brennan J, Roessler E, Waldmann T A. The interleukin (IL) 2 receptor beta chain is shared by IL-2 and a cytokine, provisionally designated IL-T, that stimulates T-cell proliferation and the induction of lymphokine-activated killer cells. Proc Natl Acad Sci USA. 1994; 91:4940-4944.
25. Burton J D, Bamford R N, Peters C, Grant A J, Kurys G, Goldman C K, Brennan J, Roessler E, Waldmann T A. A lymphokine, provisionally designated interleukin T and produced by a human adult T-cell leukemia line, stimulates T-cell proliferation and the induction of lymphokine-activated killer cells. Proc Natl Acad Sci USA. 1994; 91:4935-4939.
26. Gin J G, Ahdieh M, Eisenman J, Shanebeck K, Grabstein K, Kumaki S, Namen A, Park L S, Cosman D, Anderson D. Utilization of the beta and gamma chains of the IL-2 receptor by the novel cytokine IL-15. EMBO J. 1994; 13:2822-2830.
27. Grabowska M M, DeGraff D J, Yu X, Jin R J, Chen Z, Borowsky A D, Matusik R J. Mouse models of prostate cancer: picking the best model for the question. Cancer Metastasis Rev. 2014; 33:377-397.
28. Anguille S, Smits E L, Cools N, Goossens H, Berneman Z N, Van Tendeloo V F. Short-term cultured, interleukin-15 differentiated dendritic cells have potent immunostimulatory properties. J Transl Med. 2009; 7:109.
29. Anguille S, Lion E, Tel J, de Vries I J, Coudere K, Fromm P D, Van Tendeloo V F, Smits E L, Berneman Z N. Interleukin-15-induced CD56(+) myeloid dendritic cells combine potent tumor antigen presentation with direct tumoricidal potential. PLoS One. 2012; 7:e51851.
30. Alleva D G, Kaser S B, Monroy M A, Fenton M J, Beller D I. IL-15 functions as a potent autocrine regulator of macrophage proinflammatory cytokine production: evidence for differential receptor subunit utilization associated with stimulation or inhibition. J Immunol. 1997; 159:2941-2951.
31. Gin J G, Kumaki S, Ahdieh M, Friend D J, Loomis A, Shanebeck K, DuBose R, Cosman D, Park L S, Anderson D M. Identification and cloning of a novel IL-15 binding protein that is structurally related to the alpha chain of the IL-2 receptor. EMBO J. 1995; 14:3654-3663.
32. Dubois S, Mariner J, Waldmann T A, Tagaya Y. IL-15Ralpha recycles and presents IL-15 In trans to neighboring cells. Immunity. 2002; 17:537-547.
33. Okamura, H.; Tsutsui, H.; Komatsu, T.; Yutsudo, M.; Hakura, A.; Tanimoto, T.; Torigoe, K.; Okura, T. Nukada, Y; Hattori, K.; et al. Cloning of a new cytokine that induces IFN-production by T cells. Nature 1995, 378, 88-91.
34. Nakanishi, K.; Yoshimoto, T.; Tsutsui, H.; Okamura, H. Interleukin-18 regulates both Th1 and Th2 responses. Annu. Rev. Immunol. 2001, 19, 423-474.
35. Ushio, S.; Namba, M.; Okura, T.; Hattori, K.; Nukada, Y; Akita, K.; Tanabe, F.; Konishi, K.; Micallef, M.; Fujii, M.; et al. Cloning of the cDNA for human IFN-g-inducing factor, expression in Escherichia coli, and studies on the biologic activities of the protein. J. Immunol. 1996, 156, 4274-4279.
36. Nakanishi, K. Unique Action of Interleukin-18 on T Cells and Other Immune Cells. Front. Immunol. 2018, 9, 763.
37. Yasuda, K, Nakanishi, K, Tsutsui H. Interleukin-18 in Health and Disease. Intl J Mol Sci 2019 20:649.
38. Novick, D.; Kim, S. H.; Fantuzzi, G.; Reznikov, L. L.; Dinarello, C. A.; Rubinstein, M. Interleukin-18 binding protein: A novel modulator of the Th1 cytokine response. Immunity 1999, 10, 127-136.
39. Aizawa, Y.; Akita, K.; Taniai, M.; Torigoe, K.; Mori, T.; Nishida, Y.; Ushio, S.; Nukada, Y; Tanimoto, T.; Ikegami, H.; et al. Cloning and expression of interleukin-18 binding protein. FEBS Lett. 1999, 445, 338-342.
40. Novick, D.; Rubinstein, M. The tale of soluble receptors and binding proteins: From bench to bedside. Cytokine & Growth Factor Rev. 2007, 18, 525-533. Blood 2000; 96(4):1465-1473.
41. Namen A E. Schmierer A E, March C J, et al. B cell precursor growth promoting activity. Purification and characterization of a growth factor active on lymphocyte precursors. J. Exp. Med. 1988: 167 988-1002.
42. Onder L, Narang P, Scandella E, Chai Q, Iolyeva M, Hoorweg K, et al. IL-7-producing stromal cells are critical for lymph node remodeling. Blood (2012) 120:4675-83.
43. Link A, Vogt T K, Favre S, Britschgi M R, Acha-Orbea H, Hinz B, et al. Fibroblastic reticular cells in lymph nodes regulate the homeostasis of naive T cells. Nat Immunol. (2007) 8:1255-1265
44. Peschon J J, Morrissey P J, Grabstein K H, Ramsdell F J, Maraskovsky E, Gliniak B C, et al. Early lymphocyte expansion is severely impaired in interleukin 7 receptor-deficient mice. J Exp Med. (1994) 180:1955-60.
45. Schluns K S, Kieper W C, Jameson S C, Lefrancois L. Interleukin-7 mediates the homeostasis of naive and memory CD8 T cells in vivo. Nat Immunol. (2000) 1:426-32.
46. Tan J T, Dudl E, LeRoy E, Murray R, Sprent J, Weinberg K I, et al. IL-7 is critical for homeostatic proliferation and survival of naive T cells. Proc Natl Acad Sci USA. (2001) 98:8732-7.
47. Rathmell J C, Farkash E A, GaoW, Thompson C B. IL-7 enhances the survival and maintains the size of naive T cells. J Immunol. (2001) 167:6869-76.
48. Li J, Huston G, Swain S L. IL-7 promotes the transition of CD4 effectors to persistent memory cells. J Exp Med. (2003) 198:1807-1815.
49. Carrio R, Rolle C E, Malek T R. Non-redundant role for IL-7R signaling for the survival of CD8+ memory T cells. Eur J Immunol. (2007) 37:3078-88.
50. Carrette F, Surh C D. IL-7 signaling and CD127 receptor regulation in the control of T cell homeostasis. Semin Immunol. 2012:24; 209-217
51. Kondo M., Takeshita T, Higuchi M et al Functional participation of the IL-2 receptor gamma chain in IL-7 receptor complexes. Science 1994 263: 1453-1454.
52. Weiss J M, Subleski J J, Wigginton J M, Wiltrout R H. Immunotherapy of cancer by IL-12-based cytokine combinations. Expert Opin Biol Ther. 2007; 7:1705-1721.
53. Colombo M P, Trinchieri G. Interleukin-12 in anti-tumor immunity and immunotherapy. Cytokine Growth Factor Rev. 2002; 13:155-168.
54. Berinstein N L. Enhancing cancer vaccines with immunomodulators. Vaccine. 2007; 25 Suppl 2:B72-88.
55. Rook A H, Wood G S, Yoo E K, Elenitsas R, Kao D M, Sherman M L, Witmer W K, Rockwell K A, Shane R B, Lessin S R, Vonderheid E C. Interleukin-12 therapy of cutaneous T-cell lymphoma induces lesion regression and cytotoxic T-cell responses. Blood. 1999; 94:902-908.
56. Younes A, Pro B, Robertson M J, Flinn I W, Romaguera J E, Hagemeister F, Dang N H, Fiumara P, Loyer E M, Cabanillas F F, McLaughlin P W, Rodriguez M A, Samaniego F. Phase II clinical trial of interleukin-12 in patients with relapsed and refractory non-Hodgkin's lymphoma and Hodgkin's disease. Clin Cancer Res. 2004; 10:5432-5438.
57. Little R F, Pluda J M, Wyvill K M, Rodriguez-Chavez I R, Tosato G, Catanzaro A T, Steinberg S M, Yarchoan R. Activity of subcutaneous interleukin-12 in AIDS-related Kaposi sarcoma. Blood. 2006; 107:4650-4657.
58. Lenzi R, Edwards R, June C, Seiden M V, Garcia M E, Rosenblum M, Freedman R S. Phase II study of intraperitoneal recombinant interleukin-12 (rhIL-12) in patients with peritoneal carcinomatosis (residual disease <1 cm) associated with ovarian cancer or primary peritoneal carcinoma. J Transl Med. 2007; 5:66.
59. Anwer K, Barnes M N, Fewell J, Lewis D H, Alvarez R D. Phase-I clinical trial of IL-12 plasmid/lipopolymer complexes for the treatment of recurrent ovarian cancer. Gene Ther. 2010; 17:360-9.
60. Daud A I, DeConti R C, Andrews S, Urbas P, Riker A I, Sondak V K, Munster P N, Sullivan D M, Ugen K E, Messina J L, Heller R. Phase I trial of interleukin-12 plasmid electroporation in patients with metastatic melanoma. J Clin Oncol. 2008; 26:5896-903.
61. Mazzolini G, Alfaro C, Sangro B, Feijoo E, Ruiz J, Benito A, Tirapu I, Arina A, Sola J, Herraiz M, Lucena F, Olague C, Subtil J, Quiroga J, Herrero I, Sadaba B, Bendandi M, Qian C, Prieto J, Melero I. Intratumoral injection of dendritic cells engineered to secrete interleukin-12 by recombinant adenovirus in patients with metastatic gastrointestinal carcinomas. J Clin Oncol. 2005; 23:999-1010.
62. Zhang L, Morgan R A, Beane J D, Zheng Z, Dudley M E, Kassim S H, Nahvi A V, Ngo L T, Sherry R M, Phan G Q, Hughes M S, Kammula U S, Feldman S A, Toomey M A, Kerkar S P, Restifo N P, Yang J C, Rosenberg S A. Tumor-infiltrating lymphocytes genetically engineered with an inducible gene encoding interleukin-12 for the immunotherapy of metastatic melanoma. Clin Cancer Res. 2015; 21:2278-88.
63. Daud A I, DeConti R C, Andrews S, Urbas P, Riker A I, Sondak V K, Munster P N, Sullivan D M, Ugen K E, Messina J L, Heller R. Phase I trial of interleukin-12 plasmid electroporation in patients with metastatic melanoma. J Clin Oncol. 2008; 26:5896-5903.
64. Anwer K, Barnes M N, Fewell J, Lewis D H, Alvarez R D. Phase-I clinical trial of IL-12 plasmid/lipopolymer complexes for the treatment of recurrent ovarian cancer. Gene Ther. 2010; 17:360-369.
65. Rudman S M, Jameson M B, McKeage M J, Savage P, Jodrell D I, Harries M, Acton G, Erlandsson F, Spicer J F. A phase 1 study of AS1409, a novel antibody-cytokine fusion protein, in patients with malignant melanoma or renal cell carcinoma. Clin Cancer Res. 2011; 17:1998-2005.
66. Kikuchi T, Akasaki Y, Abe T, Fukuda T, Saotome H, Ryan J L, Kufe D W, Ohno T. Vaccination of glioma patients with fusions of dendritic and glioma cells and recombinant human interleukin 12. J Immunother. 2004; 27:452-459.
67. Zhang L, Feldman S A, Zheng Z, Chinnasamy N, Xu H, Nahvi A V, Dudley M E, Rosenberg S A, Morgan R A. Evaluation of gamma-retroviral vectors that mediate the inducible expression of IL-12 for clinical application. J Immunother. 2012; 35:430-439.
68. Thompson J A, Curti B D, Redman B G, Bhatia S, Weber J S, Agarwala S S, Sievers E L, Hughes S D, DeVries T A, Hausman D F. Phase I study of recombinant interleukin-21 in patients with metastatic melanoma and renal cell carcinoma. J Clin Oncol. 2008; 26:2034-9.
69. Davis I D, Skrumsager B K, Cebon J, Nicholaou T, Barlow J W, Moller N P, Skak K, Lundsgaard D, Frederiksen K S, Thygesen P, McArthur G A. An open-label, two-arm, phase I trial of recombinant human interleukin-21 in patients with metastatic melanoma. Clin Cancer Res. 2007; 13:3630-6.
70. Davis I D, Brady B, Kefford R F, Millward M, Cebon J, Skrumsager B K, Mouritzen U, Hansen L T, Skak K, Lundsgaard D, Frederiksen K S, Kristjansen P E, McArthur G. Clinical and biological efficacy of recombinant human interleukin-21 in patients with stage IV malignant melanoma without prior treatment: a phase IIa trial. Clin Cancer Res. 2009; 15:2123-9.
71. Petrella T M, Tozer R, Belanger K, Savage K J, Wong R, Smylie M, Kamel-Reid S, Tron V, Chen B E Hunder N N, Hagerman L, Walsh W, Eisenhauer E A. Interleukin-21 has activity in patients with metastatic melanoma: a phase II study. J Clin Oncol. 2012; 30:3396-401.
72. Ma J, Ma D, Ji C. The role of IL-21 in hematological malignancies. Cytokine. 2011; 56:133-9.
46. Hashmi M H, Van Veldhuizen P J. Interleukin-21: updated review of Phase I and II clinical trials in metastatic renal cell carcinoma, metastatic melanoma and relapsed/refractory indolent non-Hodgkin's lymphoma. Expert Opin Biol Ther. 2010; 10:807-17.
73. Coquet J M, Skak K, Davis I D, Smyth M J, Godfrey D I. IL-21 Modulates Activation of NKT Cells in Patients with Stage IV Malignant Melanoma. Clin Transl Immunology. 2013; 2:e6.
74. Bhatia S, Curti B, Ernstoff M S, Gordon M, Heath E I, Miller W H, Jr., Puzanov I, Quinn D I, Flaig T W, VanVeldhuizen P, Byrnes-Blake K, Freeman J A, Bittner R, Hunder N, Souza S, Thompson J A. Recombinant interleukin-21 plus sorafenib for metastatic renal cell carcinoma: a phase 1/2 study. J Immunother Cancer. 2014; 2:2.
75. Timmerman J M, Byrd J C, Andorsky D J, Yamada R E, Kramer J, Muthusamy N, Hunder N, Pagel T M. A phase I dose-finding trial of recombinant interleukin-21 and rituximab in relapsed and refractory low grade B-cell lymphoproliferative disorders. Clin Cancer Res. 2012; 18:5752-60.
76. Gowda A, Roda J, Hussain S R, Ramanunni A, Joshi T, Schmidt S, Zhang X, Lehman A, Jarjoura D, Carson W E, Kindsvogel W, Cheney C, Caligiuri M A, Tridandapani S, Muthusamy N, Byrd J C. IL-21 mediates apoptosis through up-regulation of the BH3 family member BIM and enhances both direct and antibody-dependent cellular cytotoxicity in primary chronic lymphocytic leukemia cells in vitro. Blood. 2008; 111:4723-30.
77. Browning R L, Byrd W H, Gupta N, Jones J, Mo X, Hertlein E, Yu L, Muthusamy N, Byrd J C. Lenalidomide Induces Interleukin-21 Production by T Cells and Enhances IL21-Mediated Cytotoxicity in Chronic Lymphocytic Leukemia B Cells. Cancer Immunol Res. 2016; 4:698-707.
78. Morris J C, Ramlogan-Steel C A, Yu P, Black B A, Mannan P, Allison J P, Waldmann T A, Steel J C. Vaccination with tumor cells expressing IL-15 and IL-15Ralpha inhibits murine breast and prostate cancer. Gene Ther. 2014; 21:393-401.
79. Yu P, Steel J C, Zhang M, Morris J C, Waldmann T A. Simultaneous blockade of multiple immune system inhibitory checkpoints enhances antitumor activity mediated by interleukin-15 in a murine metastatic colon carcinoma model. Clin Cancer Res. 2010; 16:6019-6028.
80. Lasek W, Golab J, Maslinski W, Switaj T, Balkowiec E Z, Stoklosa T, Giermasz A, Malejczyk M, Jakobisiak M. Subtherapeutic doses of interleukin-15 augment the antitumor effect of interleukin-12 in a B16F10 melanoma model in mice. Eur Cytokine Netw. 1999; 10:345-356.
81. Comes A, Di Carlo E, Musiani P, Rosso O, Meazza R, Chiodoni C, Colombo M P, Ferrini S. IFN-gamma-independent synergistic effects of IL-12 and IL-15 induce anti-tumor immune responses in syngeneic mice. Eur J Immunol. 2002; 32:1914-1923.
82. Osaki T, Peron J M, Cai Q, et al. IFN-gamma-inducing factor/IL-18 administration mediates IFNgamma- and IL-12-independent antitumor effects. J Immunol 1998; 160(4):1742-1749.
83. Vidal-Vanaclocha F, Mendoza L, Telleria N, et al. Clinical and experimental approaches to the pathophysiology of interleukin-18 in cancer progression. Cancer Metastasis Rev 2006; 25(3):417-434.
84. Coughlin C M, Salhany K E, Wysocka M, et al. Interleukin-12 and interleukin-18 synergistically induce murine tumor regression which involves inhibition of angiogenesis. J Clin Invest 1998; 101 (6):1441-1452
85. Li Q, Carr A L, Donald E J, et al. Synergistic effects of IL-12 and IL-18 in skewing tumor-reactive Tcell responses towards a type 1 pattern. Cancer Res 2005; 65(3):1063-1070. [PubMed: 15705908
86. Ma, Z.; Li, W.; Yoshiya, S.; Xu, Y.; Hata, M.; El-Darawish, Y.; Markova, T.; Yamanishi, K.; Yamanishi, H.; Tahara, H.; et al. Augmentation of Immune Checkpoint Cancer Immunotherapy with IL18. Clin. Cancer Res. 2016,22, 2969-2980.
87 Hu, B.; Ren, J.; Luo, Y.; Keith, B.; Young, R. M.; Scholler, J.; Zhao, Y.; June, C. H. Augmentation of Antitumor Immunity by Human and Mouse CART Cells Secreting IL-18. Cell Rep. 2017, 20, 3025-3033.
88. Carson W E, Dierksheide J E, Jabbour S, et al. Coadministration of interleukin-18 and interleukin-12 induces a fatal inflammatory response in mice: critical role of natural killer cell interferon-gamma production and STAT-mediated signal transduction.
89. Robertson, M. J.; Mier, J. W.; Logan, T.; Atkins, M.; Koon, H.; Koch, K. M.; Kathman, S; Pandite, L. N.; Oei, C.; Kirby, L. C.; et al. Clinical and biological effects of recombinant human interleukin-18 administered by intravenous infusion to patients with advanced cancer. Clin. Cancer Res. 2006, 12, 4265-4273.
90. Robertson, M. J.; Kirkwood, J. M.; Logan, T. F.; Koch, K. M.; Kathman, S; Kirby, L. C.; Bell, W. N.; Thurmond, L. M.; Weisenbach, J.; Dar, M. M. A dose-escalation study of recombinant human interleukin-18 using two different schedules of administration in patients with cancer. Clin Cancer Res. 2008, 14, 3462-3469.
91. Robertson, M. J.; Kline, J.; Struemper, H.; Koch, K. M.; Bauman, J. W.; Gardner, O. S.; Murray, S. C.; Germaschewski, F.; Weisenbach, J.; Jonak, Z.; et al. A dose-escalation study of recombinant human interleukin-18 in combination with rituximab in patients with non-Hodgkin lymphoma. J. Immunother. 2013, 36, 331-341.
92. Jicha D L, Mule J J, Rosenberg S A. Interleukin 7 generates antitumor cytotoxic T lymphocytes against murine sarcomas with efficacy in cellular adoptive immunotherapy. J Exp Med 1991; 174(6):1511-1515.
93. Komschlies K L, Gregorio T A, Gruys M E, Back T C, Faltynek C R, Wiltrout R H. Administration of recombinant human IL-7 to mice alters the composition of B-lineage cells and T cell subsets, enhances T cell function, and induces regression of established metastases. J Immunol 1994; 152(12):5776-5784.
94. Ptackova P, Musil J, Stach M, Lesny P, Nemeckova S, Kral V, et al. A new approach to CAR T-cell gene engineering and cultivation using piggyBac transposon in the presence of IL-4, IL-7 and IL-21. Cytotherapy 2018 20:507-520.
95. Rosenberg S A, Sportes C, Ahmadzadeh M, Fry T J, Ngo L T, Schwarz S L, et al. IL-7 administration to humans leads to expansion of CD8+ and CD4+ cells but a relative decrease of CD4+ T-regulatory cells. J Immunother (2006) 29:313-19.
96. Sportes C, Hakim F T, Memon S A, Zhang H, Chua K S, Brown M R, et al Administration of rhIL-7 in humans increases in vivo TCR repertoire diversity by preferential expansion of naive T cell subsets. J Exp Med. (2008) 205:1701-14.
97 Wallinngton J C Williams A P Staples K J Wilkinson T M S. IL-12 and IL-7 synergize to control mucosal-associated invariant T cell cytotoxic responses to bacterial infection. J Allergy Clin Immunol 2018 141: 2182-2195
98 Nakao S, Arai Y, Tasaki M, Yamashita M, Murakami R, Kawase T, Amino N, Nakatake M, Kurosaki H, Mori M, Takeuchi M, Nakamura T. Intratumoral expression of IL-7 and IL-12 using an oncolytic virus increases systemic sensitivity to immune checkpoint blockade. Sci Trans Med 12 (526) 2020
99. Kerkela R, Grazette L, Yacobi R, Iliescu C, Patten R, Beahm C, Walters B, Shevtsov S, Pesant S, Clubb F J, Rosenzweig A, Salomon R N, Van Etten R A, Alroy J, Durand J B and Force T. Cardiotoxicity of the cancer therapeutic agent imatinib mesylate. Nat. Med. 12:908-916 (2006).
100. Portielje J E, Gratama J W, van Ojik H H, Stoter G and Kruit W H. IL-12: a promising adjuvant for cancer vaccination. Cancer Immunol. Immunother. 52:133-144 (2003).
101. Sasiain M C, de la Barrera S, Fink S, Finiasz M, Aleman M, Farina M H, Pizzariello G and Valdez R. Interferon-gamma (IFN-gamma) and tumour necrosis factor-alpha (TNF-alpha) are necessary in the early stages of induction of CD4 and CD8 cytotoxic T cells by Mycobacterium leprae heat shock protein (hsp) 65 kD. Clin. Exp. Immunol. 114:196-203 (1998).
102. Portielje J E, Lamers C H, Kruit W H, Sparreboom A, Bolhuis R L, Stoter G, Huber C and Gratama J W. Repeated administrations of interleukin (IL)-12 are associated with persistently elevated plasma levels of IL-10 and declining IFN-gamma, tumor necrosis factor-alpha, IL-6, and IL-8 responses. Clin. Cancer Res. 9:76-83 (2003).
103. Leonard J P, Sherman M L, Fisher G L, Buchanan L J, Larsen G, Atkins M B, Sosman J A, Dutcher J P, Vogelzang N J and Ryan J L. Effects of single-dose interleukin-12 exposure on interleukin-12-associated toxicity and interferon-gamma production. Blood. 90:2541-2548 (1997).
104. Sacco S, Heremans H, Echtenacher B, Buurman W A, Amraoui Z, Goldman M and Ghezzi P. Protective effect of a single interleukin-12 (IL-12) predose against the toxicity of subsequent chronic IL-12 in mice: role of cytokines and glucocorticoids. Blood. 90:4473-4479 (1997).
105. Masztalerz A, Van Rooijen N, Den Otter W and Everse L A. Mechanisms of macrophage cytotoxicity in IL-2 and IL-12 mediated tumour regression. Cancer Immunol. Immunother. 52:235-242 (2003).
106. Zagozdzon R, Golab J, Stoklosa T, Giermasz A, Nowicka D, Feleszko W, Lasek W and Jakobisiak M. Effective chemo-immunotherapy of L1210 leukemia in vivo using interleukin-12 combined with doxorubicin but not with cyclophosphamide, paclitaxel or cisplatin. Int. J. Cancer. 77:720-727 (1998).
107. Tatsumi T, Takehara T, Kanto T, Miyagi T, Kuzushita N, Sugimoto Y, Jinushi M, Kasahara A, Sasaki Y, Hori M and Hayashi N. Administration of interleukin-12 enhances the therapeutic efficacy of dendritic cell-based tumor vaccines in mouse hepatocellular carcinoma. Cancer Res. 61:7563-7567 (2001).
108. Nastala C L, Edington H D, McKinney T G, Tahara H, Nalesnik M A, Brunda M J, Gately M K, Wolf S F, Schreiber R D and Storkus W J. Recombinant IL-12 administration induces tumor regression in association with IFN-gamma production. J. Immunol. 153:1697-1706 (1994).
109. Dunussi-Joannopoulos K, Runyon K, Erickson J, Schaub R G, Hawley R G and Leonard J P. Vaccines with interleukin-12-transduced acute myeloid leukemia cells elicit very potent therapeutic and long-lasting protective immunity. Blood. 94:4263-4273 (1999).
110. Coughlin C M, Wysocka M, Trinchieri G and Lee W M. The effect of interleukin 12 desensitization on the antitumor efficacy of recombinant interleukin 12. Cancer Res. 57:2460-2467 (1997).
111. Asselin-Paturel C, Megherat S, Vergnon I, Echchakir H, Dorothee G, Blesson S, Gay F, Mami-Chouaib F and Chouaib S. Differential effect of high doses versus low doses of interleukin-12 on the adoptive transfer of human specific cytotoxic T lymphocyte in autologous lung tumors engrafted into severe combined immunodeficiency disease-nonobese diabetic mice: relation with interleukin-10 induction. Cancer. 91:113-122 (2001).
112. Gollob J A, Mier J W, Veenstra K, McDermott D F, Clancy D, Clancy M and Atkins M B. Phase I trial of twice-weekly intravenous interleukin 12 in patients with metastatic renal cell cancer or malignant melanoma: ability to maintain IFN-gamma induction is associated with clinical response. Clin. Cancer Res. 6:1678-1692 (2000).
113. Hoshino T, Jiang Y Z, Dunn D, Paul D, Qazilbash M, Cowan K, Wang J, Barrett J and Liu J. Transfection of interleukin-12 cDNAs into tumor cells induces cytotoxic immune responses against native tumor: implications for tumor vaccination. Cancer Gene Ther. 5:150-157 (1998).
114. Vigna E and Naldini L. Lentiviral vectors: excellent tools for experimental gene transfer and promising candidates for gene therapy. J. Gene Med. 2:308-316 (2000).
115. Logan A C, Lutzko C and Kohn D B. Advances in lentiviral vector design for gene-modification of hematopoietic stem cells. Curr. Opin. Biotechnol. 13:429-436 (2002).
116. Silvertown J D, Symes J C, Neschadim A, Nonaka T, Kao J C, Summerlee A J and Medin J A. Analog of H2 relaxin exhibits antagonistic properties and impairs prostate tumor growth. FASEB J. 21:754-765 (2007).
117. Yoshimitsu M, Sato T, Tao K, Walia J S, Rasaiah V I, Sleep G T, Murray G J, Poeppl A G, Underwood J, West L, Brady R O and Medin J A. Bioluminescent imaging of a marking transgene and correction of Fabry mice by neonatal injection of recombinant lentiviral vectors. Proc. Natl. Acad. Sci. U.S.A. 101:16909-16914 (2004).
118. Sato T, Neschadim A, Konrad M, Fowler D H, Lavie A and Medin J A. Engineered human tmpk/AZT as a novel enzyme/prodrug axis for suicide gene therapy. Mol. Ther. 15:962-970 (2007).
119. Baum C. I could die for you: new prospects for suicide in gene therapy. Mol. Ther. 15:848-849 (2007).
120. Levine B L, Humeau L M, Boyer J, MacGregor R R, Rebello T, Lu X, Binder G K, Slepushkin V, Lemiale F, Mascola J R, Bushman F D, Dropulic B and June C H. Gene transfer in humans using a conditionally replicating lentiviral vector. Proc. Natl. Acad. Sci. U.S.A. 103:17372-17377 (2006).
121. Yoshimitsu M, Higuchi K, Ramsubir S, Nonaka T, Rasaiah V I, Siatskas C, Liang S B, Murray G J, Brady R O and Medin J A. Efficient correction of Fabry mice and patient cells mediated by lentiviral transduction of hematopoietic stem/progenitor cells. Gene Ther. 14:256-265 (2007).
122. Dessureault S, Noyes D, Lee D, Dunn M, Janssen W, Cantor A, Sotomayor E, Messina J and Antonia S J. A phase-1 trial using a universal GM-CSF-producing and CD40L-expressing bystander cell line (GM.CD40L) in the formulation of autologous tumor cell-based vaccines for cancer patients with stage IV disease. Ann. Surq. Oncol. 14:869-884 (2007).
123. Vereecque R, Saudemont A, Wickham T J, Gonzalez R, Hetuin D, Fenaux P and Quesnel B. Gamma-irradiation enhances transgene expression in leukemic cells. Gene Ther. 10:227-233 (2003).
124. Eisterer W, Jiang X, Christ O, Glimm H, Lee K H, Pang E, Lambie K, Shaw G, Holyoake T L, Petzer A L, Auewarakul C, Barnett M J, Eaves C J and Eaves A C. Different subsets of primary chronic myeloid leukemia stem cells engraft immunodeficient mice and produce a model of the human disease. Leukemia. 19:435-441 (2005).
125. National Cancer Institute. SEER Cancer Statistics Review. 2007: (2007). http://seer.cancer.gov/
126. Seiter K. Acute Lymphoblastic Leukemia. (2006). http://www.emedicine.com/med/topic3146.htm
127. Redaelli A, Stephens J M, Laskin B L, Pashos C L and Botteman M F. The burden and outcomes associated with four leukemias: AML, ALL, CLL and CML. Expert Rev. Anticancer Ther. 3:311-329 (2003).
128. Piccaluga P P, Martinelli G and Baccarani M. Advances in the treatment for haematological malignancies. Expert Opin. Pharmacother. 7:721-732 (2006).
129. Dasatinib (Sprycel®) Drug Product Label.
130. Nilotinib (Tasigna®) Drug Product Label.
131. Igarashi Y, Sasada T. Cancer Vaccines: Toward the Next Breakthrough in Cancer Immunotherapy. J Immunol Res. 2020 Nov. 17; 2020:5825401. doi: 10.1155/2020/5825401. PMID: 33282961; PMCID: PMC7685825.
132. Srivatsan S, Patel J M, Bozeman E N, Imasuen I E, He S, Daniels D, Selvaraj P. Allogeneic tumor cell vaccines: the promise and limitations in clinical trials. Hum Vaccin Immunother. 2014; 10(1):52-63. doi: 10.4161/hv.26568. Epub 2013 Sep. 24. PMID: 24064957; PMCID: PMC4181031.
133. VanderVeen N, Raja N, Yi E, Appelman H, Ng P, Palmer D, Zamler D, Dzaman M, Lowenstein P R, Castro M G. Preclinical Efficacy and Safety Profile of Allometrically Scaled Doses of Doxycycline Used to Turn “On” Therapeutic Transgene Expression from High-Capacity Adenoviral Vectors in a Glioma Model. Hum Gene Ther Methods. 2016 June; 27(3):98-111. doi: 10.1089/hgtb.2015.168. Epub 2016 Apr. 28. PMID: 27056322; PMCID: PMC4926229.
134. Becskei A. Tuning up Transcription Factors for Therapy. Molecules. 2020 Apr. 20; 25(8):1902. doi: 10.3390/molecules25081902. PMID: 32326099; PMCID: PMC7221782.
135 Paige, C. J., Kincade, P. W., and Ralph, P. (1978). Murine B cell leukemia line with inducible surface immunoglobulin expression. J Immunol 121: 641-647.
136 Dull, T., et al. (1998). A third-generation lentivirus vector with a conditional packaging system. J Virol 72: 8463-8471.
137 Silvertown, J. D., Walia, J. S., Summerlee, A. J., and Medin, J. A. (2006). Functional expression of mouse relaxin and mouse relaxin-3 in the lung from an Ebola virus glycoprotein-pseudotyped lentivirus via tracheal delivery. Endocrinology 147: 3797-3808.
138. Reiser J, Lai Z, Zhang X Y, Brady R O. Development of multigene and regulated lentivirus vectors. J. Virology. 2000; 74:10589-10599.
139. Fernandez A, Guzman S, Cruz Y, Zamorano P. Construction of bcistronic lentiviral vectors for tracking the expression of CDNF in transduced cells. Plasmid. 2014; 76:15-23.
140. Huss D, Lansford R. Generation and analysis of lentivirus expressing a 2A peptide linked bicistronic fluorescent construct. Cold Spring Harb Protoc. 2014; 12:1290-1311.
141. Liu A, Chen O, Wall J B J, Zheng M, Zhou Y, Wang I, Vaseghi H R, Qian L, Liu J. Systemic comparison of 2A peptides for cloning multi-genes in a polycistronic vector. Scientific Reports. 2016; 7:2193-2202.
142. Konforte D, Paige C J. Identification of cellular intermediates and molecular pathways induced by IL-12 in human B cells. 2006; 177:8381-8392.
143. Arber C, Abhyankar H, Heslop H E, Brenner M K, Liu H, Dotti G, Savoldo B. The immunogenicity of virus-derived 2A sequences in immunocompetent individuals. Gene Ther. 2013. 20; 958-962.
144. Scaife, M, Pacienza, N, Au B C, Wang J C, Devine, S, Scheid, E et al. (2013). Engineered human Tmpk fused with truncated cell-surface markers: versatile cell-fate control safety cassettes. Gene Ther 20: 24-34.
145. Barrett et al. Regulated intratumoral expression of IL-12 using a RheoSwitch Therapeutic System® (RTS®) gene switch as gene therapy for the treatment of glioma. Cancer Gene Therapy (2018) 25:106-116
146. Chiocca et al. Regulatable interleukin-12 gene therapy in patients with recurrent high-grade glioma: Results of a phase 1 trial. Sci Transl Med. 2019 Aug. 14; 11(505)

1. A multicytokine lentiviral construct or composition comprising a lentiviral vector;
an IL-12 expression cassette; and
an IL-21 expression cassette and/or an IL-18 expression cassette;
optionally wherein the IL-21 expression cassette and the IL-12 expression cassette form an IL-12-IL-21 expression cassette or IL-21-IL-12 expression cassette or the IL-18 expression cassette and the IL-12 expression cassette form an IL-12-IL-18 expression cassette or IL-18-IL-12 expression cassette.

2. The vector construct or composition of claim 1, wherein the IL-12 expression cassette comprises a polynucleotide encoding a p35 polypeptide and a polynucleotide encoding a p40 polypeptide; or a polynucleotide encoding an IL-12 fusion polypeptide.

3. The vector construct or composition of claim 2 wherein the polynucleotide encoding the IL-12 fusion polypeptide has at least 70% sequence identity to SEQ ID NO: 4 and binds an IL-12 receptor; and/or wherein
i) one or more of the expression cassettes comprises an IL-2 signal sequence, preferably human;
ii) the IL-21 expression cassette encodes an IL-21 polypeptide having at least 70% sequence identity to SEQ ID NO: 7 and binds an IL-21 receptor, or the IL-18 expression cassette encodes an IL-18 polypeptide having at least 70% sequence identity to SEQ ID NO: 6 and binds an IL-18 receptor;
iii) one or more of the IL-12 expression cassette, and the IL-18 expression cassette and/or the IL-21 expression cassette comprises an inducible promoter;
iv) the lentiviral vector is a clinical grade vector;
v) one or more of the expression cassettes, optionally comprising IL-18, comprises a IL-2 signal sequence, preferably human; and/or
vi) the construct comprises a cell fate control component, preferably a tmpk cassette.

4.-7. (canceled)

8. The vector construct or composition of claim 1, wherein the lentiviral vector is a clinical grade vector.

9. The composition of claim 1, wherein the composition is a pharmaceutical composition and further comprises a pharmaceutically acceptable carrier.

10. (canceled)

11. An isolated virus comprising the vector construct or the composition of claim 1, preferably a lentivirus or adenovirus or adeno associated virus.

12. An isolated cell secreting IL-12 and at least one of IL-21 and/or IL-18 at the or above a threshold level, wherein the cell is transduced with the vector construct or composition of claim 1 or an isolated virus comprising the vector construct or composition.

13. The isolated cell of claim 12 wherein the cell is a cancer cell, optionally preferably an established cell line, a primary cancer cell, or a cancer cell derived from a subject, and/or wherein the cancer cell is a leukemic cell, preferably an ALL cell, an AML cell or a CLL cell, lymphoma cell, myeloma cell, glioblastoma cell, melanoma cell, or cancer cell of the lung, ovary, prostate, breast, colon, bladder, liver, pancreas, thyroid, or head and neck, and/or wherein the IL-12 is secreted at a ratio of 10:1, 5:1, 2:1, or 1:1 relative to IL-18 or IL-21.

14.-15. (canceled)

16. A population of cells comprising isolated cells of claim 12 wherein the population of cells comprises at least 0.1 to 50% IL-12 and at least one of IL-21 and/or IL-18 producing cells, preferably 0.5% to about 40%, about 0.5%, about 1%, about 1-5%, 5-10%, 10-40% or more IL-12 and at least one of IL-21 and/or IL-18 producing cells, and wherein the population of cells secretes IL-12 and at least one of IL-21 and/or IL-18 levels, for example at a level to induce or enhance an immune response, preferably a CD4+ T cell dependent immune response.

17. A whole cell vaccine comprising the isolated cell or population of cells of any claim 12 and optionally an adjuvant.

18. A composition comprising
a) the vector construct of claim 1,
b) an isolated virus comprising the vector construct, preferably a lentivirus or adenovirus or adeno associated virus,
c) an isolated cell secreting IL-12 and at least one of IL-18 and/or IL-21 at the or above a threshold level wherein the cell is transduced with the vector construct or an isolated virus comprising the vector construct,
d) a population of cells comprising
i) the isolated cells,
ii) at least 0.1 to 50% IL-12, and
iii) at least one of IL-18 and/or IL-21 producing cells, preferably 0.5% to about 40%, about 0.5%, about 1%, about 1-5%, 5-10%, 10-40% or more IL-12 and at least one of IL-18 and/or IL-21 producing cells, and wherein the population of cells secretes IL-12 and at least one of IL-18 and/or IL-21 levels, for example at a level to induce or enhance an immune response, preferably a CD4+ T cell dependent immune response, or
e) a whole cell vaccine comprising the isolated cell or population of cells and optionally adjuvant;
optionally wherein the composition is a pharmaceutical composition and further comprises a pharmaceutically acceptable carrier.

19. A method of expressing IL-12 and at least one of IL-18 and/or IL-21 in a cell, preferably a cancer cell, comprising contacting the cell with a composition, the vector construct of claim 1, or an isolated virus comprising the vector construct, preferably a lentivirus or adenovirus or adeno associated virus, under conditions that permit transduction of the cell, thereby providing a transduced cell, wherein the IL-12, IL-18, and/or IL-21 is secreted,
wherein the composition comprises
a) the vector construct,
b) an isolated virus comprising the vector construct, preferably a lentivirus or adenovirus or adeno associated virus,
c) an isolated cell secreting IL-12 and at least one of IL-18 and/or IL-21 at the or above a threshold level wherein the cell is transduced with the vector construct or an isolated virus comprising the vector construct,
d) a population of cells comprising
i) the isolated cells,
ii) at least 0.1 to 50% IL-12 producing cells, and
iii) at least one of IL-18 and/or IL-21 producing cells, preferably 0.5% to about 40%, about 0.5%, about 1%, about 1-5%, 5-10%, 10-40% or more IL-12 and at least one of IL-18 and/or IL-21 producing cells, and wherein the population of cells secretes IL-12 and at least one of IL-18 and/or IL-21 levels, for example at a level to induce or enhance an immune response, preferably a CD4+ T cell dependent immune response, or
e) a whole cell vaccine comprising the isolated cell or population of cells and optionally adjuvant;
optionally wherein the composition is a pharmaceutical composition and further comprises a pharmaceutically acceptable carrier.

20. The method of claim 19, further comprising a step of isolating the transduced cell or isolating a population of cells comprising the transduced cell, and/or comprising:
a) growth arresting the transduced cell, the population of cells or composition; and
b) introducing the transduced cell, population of cells and/or composition in a subject.

21. (canceled)

22. A method of reducing the number of tumor cells or cancer burden in a subject in need thereof and/or for treating a subject with cancer or an increased risk of cancer and/or inducing or enhancing an immune response or a memory immune response in a subject, optionally with cancer or an increased risk of cancer, comprising administering to the subject a vector construct, an isolated virus, an isolated cell, a transduced cell, a population of cells, a whole cell vaccine or a composition, wherein
a) the vector construct is the vector construct or composition of claim 1,
b) the isolated virus comprises the vector construct, and is preferably a lentivirus or adenovirus or adeno associated virus,
c) the isolated cell secretes IL-12 and at least one of IL-18 and/or IL-21 at the or above a threshold level and the cell is transduced with the vector construct or an isolated virus comprising the vector construct,
d) the population of cells comprises
i) the isolated cells,
ii) at least 0.1 to 50% IL-12, and
iii) at least one of IL-18 and/or IL-21 producing cells, preferably 0.5% to about 40%, about 0.5%, about 1%, about 1-5%, 5-10%, 10-40% or more IL-12 and at least one of IL-18 and/or IL-21 producing cells, and wherein the population of cells secretes IL-12 and at least one of IL-18 and/or IL-21 levels, for example at a level to induce or enhance an immune response, preferably a CD4+ T cell dependent immune response,
e) the whole cell vaccine comprises the isolated cell or population of cells and optionally adjuvant, and
f) the composition comprises the vector construct, the isolated virus, the isolated cell, the population of cells, or the whole cell vaccine, optionally wherein the composition is a pharmaceutical composition and further comprises a pharmaceutically acceptable carrier;
optionally, wherein
i) the transduced cell is produced by a method of expressing IL-12 and at least one of IL-18 and/or IL-21 in a cell, optionally a cancer cell, comprising contacting the cell with the composition, the vector construct, or the isolated virus under conditions that permit transduction of the cell, thereby providing a transduced cell, wherein the IL-12, IL-18, and/or IL-21 is secreted;
ii) the number of cells or population of cells ranges from 10⁵ cells to 10⁹ cells, preferably about 10⁵ cells, about 10⁶ cells, about 10⁷, cells, about 10⁸ cells, or about 10⁹ cells;
iii) the transduced cell, the population of cells or composition is growth arrested or irradiated and introduced in a subject; and/or
iv) the transduced cell is a cancer cell, preferably derived from the subject with cancer, optionally wherein the cancer cell is a leukemic cell, preferably an ALL cell, an AML cell or a CLL cell, lymphoma cell, myeloma cell, glioblastoma cell, melanoma cell, or cancer cell of the lung, ovary, prostate, breast, colon, bladder, liver, pancreas, thyroid, or head and neck.

23. The method of claim 22 for treating a subject with cancer or an increased risk of cancer, wherein the cancer is leukemia, preferably ALL, AML, CML or CLL, lymphoma, myeloma, glioblastoma, melanoma, or cancer of the lung, ovary, prostate, breast, colon, bladder, liver, pancreas, thyroid, or head and neck.

24. The method of claim 23 further comprising monitoring cancer progression.

25. (canceled)

26. The method of claim 22, wherein the immune response or memory immune response is initiated against a leukemia, preferably ALL, AML, CML or CLL, lymphoma, myeloma, glioblastoma, melanoma, or cancer of the lung, ovary, prostate, breast, colon, bladder, liver, pancreas, thyroid, or head and neck.

27.-65. (canceled)

66. The composition of claim 18, wherein the isolated cell is a cancer cell, preferably an established cell line, a primary cancer cell, or a cancer cell derived from a subject, and/or wherein the cancer cell is a leukemic cell, preferably an ALL cell, an AML cell or a CLL cell, lymphoma cell, myeloma cell, glioblastoma cell, melanoma cell, or cancer cell of the lung, ovary, prostate, breast, colon, bladder, liver, pancreas, thyroid, or head and neck, and/or wherein the IL-12 is secreted at a ratio of 10:1, 5:1, 2:1, or 1:1 relative to IL-18 or IL-21.

67. The method of claim 19, wherein the isolated cell is a cancer cell, preferably an established cell line, a primary cancer cell, or a cancer cell derived from a subject, and/or wherein the cancer cell is a leukemic cell, preferably an ALL cell, an AML cell or a CLL cell, lymphoma cell, myeloma cell, glioblastoma cell, melanoma cell, or cancer cell of the lung, ovary, prostate, breast, colon, bladder, liver, pancreas, thyroid, or head and neck, and/or wherein the IL-12 is secreted at a ratio of 10:1, 5:1, 2:1, or 1:1 relative to IL-18 or IL-21.

68. The method of claim 20, wherein the isolated cell is a cancer cell, preferably an established cell line, a primary cancer cell, or a cancer cell derived from a subject, and/or wherein the cancer cell is a leukemic cell, preferably an ALL cell, an AML cell or a CLL cell, lymphoma cell, myeloma cell, glioblastoma cell, melanoma cell, or cancer cell of the lung, ovary, prostate, breast, colon, bladder, liver, pancreas, thyroid, or head and neck, and/or wherein the IL-12 is secreted at a ratio of 10:1, 5:1, 2:1, or 1:1 relative to IL-18 or IL-21.


SEQUENCE LISTING (18 sequences)

SEQ ID NO: 1 - DNA, 39 bp, Artificial Sequence
  Synthetic Construct
ttggcgcgccttaggaagcattcagatagctcatcactc

SEQ ID NO: 2 - DNA, 118 bp, Artificial Sequence
  Synthetic Construct
ttttaaaagaaaaggggggattggggggtacagtgcaggggaaagaatagtagacataatagcaacagacatacaaactaaagaattacaaaaacaaattacaaaaattcaaaatttt

SEQ ID NO: 3 - DNA, 592 bp, Artificial Sequence
  Synthetic Construct
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 4 - DNA, 1611 bp, Artificial Sequence
  Synthetic Construct
atgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgttcctggagtaggggtacctggggtgggcgccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagactcgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 5 - DNA, 1623 bp, Artificial Sequence
  Synthetic Construct
atgggtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaagctgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccgttcctggagtaggggtacctggagtgggcggatctatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagtccccccgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcctag

SEQ ID NO: 6 - protein, 193 aa, Homo Sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 7 - protein, 162 aa, Homo Sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 8 - DNA, 43 bp, Artificial Sequence
  Synthetic Construct
tagctctagaggatccgccaccatggagaggacccttgtctgt

SEQ ID NO: 9 - DNA, 34 bp, Artificial Sequence
  Synthetic Construct
gaggttgattgtcgacctaggagagatgctgaat

SEQ ID NO: 10 - DNA, 108 bp, Artificial Sequence
  Synthetic Construct
agctctagaggatccgccaccatggagacagacacactcctgctatgggtactgctgctctgggttccaggttccactggtgacaactttggccgacttcactgtaca

SEQ ID NO: 11 - DNA, 40 bp, Artificial Sequence
  Synthetic Construct
gaggttgattgtcgacctaactttgatgtaagttagtgag

SEQ ID NO: 12 - DNA, 106 bp, Artificial Sequence
  Synthetic Construct
tagctctagaggatccgccaccatgtacaggatgcaactcctgtcttgcattgcactaagtcttgcacttgtcacgaattcgaactttggccgacttcactgtaca

SEQ ID NO: 13 - DNA, 40 bp, Artificial Sequence; identical to SEQ ID NO: 11
  Synthetic Construct

SEQ ID NO: 14 - DNA, 43 bp, Artificial Sequence
  Synthetic Construct
tagctctagaggatccgccaccatgttccatgtttcttttaga

SEQ ID NO: 15 - DNA, 37 bp, Artificial Sequence
  Synthetic Construct
gaggttgattgtcgacttatatactgcccttcaaaat

SEQ ID NO: 16 - DNA, 26 bp, Artificial Sequence
  Synthetic Construct
ttggcgcgccaccatgggtcaccagc

SEQ ID NO: 17 - DNA, 60 bp, Homo Sapiens
atgtacaggatgcaactcctgtcttgcattgcactaagtcttgcacttgtcacgaattcg

SEQ ID NO: 18 - protein, 20 aa, Homo Sapiens
MYRMQLLSCIALSLALVTNS